(19) United States
(12) Patent Application Publication
(10) Pub. No.: US 2021/0187106 A1
(43) Pub. Date: Jun. 24, 2021

(54) COMBINATION OF LIF INHIBITORS AND PLATINUM-BASED ANTINEOPLASTIC AGENTS FOR USE IN TREATING CANCER

(71) Applicants: MEDIMMUNE LIMITED, Cambridge (GB); FUNDACIO PRIVADA INSTITUT D'INVESTIGACIO ONCOLOGICA DE VALL HEBRON, Barcelona (ES); FUNDACIO PRIVADA INSTITUCIO CATALANA DE RECERCA I ESTUDIS AVANCATS, Barcelona (ES)

(72) Inventors: Joan SEOANE SUAREZ, Barcelona (ES); Judit ANIDO FOLGUEIRA, Barcelona (ES); Robin Matthew HALLETT, Toronto, ON (CA); Patricia Anne GIBLIN, Boston, MA (US); Jeanne MAGRAM, Boston, MA (US); Naimish PANDYA, Boston, MA (US); Robert WASSERMAN, Boston, MA (US); Angus SINCLAIR, Boston, MA (US)

(21) Appl. No.: 17/252,449

(22) PCT Filed: Jun. 17, 2019
(86) PCT No.: PCT/IB2019/000812

§ 371 Date: Dec. 15, 2020

(30) Foreign Application Priority Data
Jun. 18, 2018 (EP) 18382432.5

(51) Int. Cl. A61K 39/395; A61K 33/243; A61K 31/282; A61P 35/00

(52) U.S. Cl. CPC A61K 39/3955; A61K 33/243; A61K 2039/545; A61P 35/00; A61K 31/282

(57) ABSTRACT

Described herein are methods of treating cancer using combinations of Leukemia Inhibitory Factor (LIF)-binding polypeptides and platinum-based antineoplastic agents.

### CROSS REFERENCE TO RELATED APPLICATIONS

The present application claims the benefit of European Application Serial Number 18382432.5, filed Jun. 18, 2018, the contents of which is hereby incorporated by reference in its entirety.

### BACKGROUND

Leukemia inhibitory factor (LIF) is an Interleukin 6 (IL-6)-type cytokine that is involved in a variety of biological activities including the inhibition of cell differentiation. Human LIF is a polypeptide of 202 amino acids that exerts biological effects via binding to the cell surface LIF receptor (LIFR or CD118) which heterodimerizes with gp130. This leads to activation of pro-growth signaling pathways such as the mitogen activated protein kinase (MAPK) and the Janus activated kinase (JAK/STAT) pathway. High expression levels and high serum levels of LIF have been demonstrated to be associated with a poor prognosis for many types of cancer.

Platinum-based antineoplastic agents, also known as platins, are coordination complexes of platinum that have shown utility in the treatment of several cancers. Mechanistically, platinum-based antineoplastic agents cause cell-death by crosslinking DNA or intercalating with DNA. This inhibits DNA repair and synthesis in cells. Ultimately these cellular processes are corrupted leading to initiation of apoptosis. Common platinum-based antineoplastic agents may include cisplatin, carboplatin, oxaliplatin, nedaplatin, triplatin tetranitrate, phenathriplatin, picoplatin, satraplatin, lobaplatin, or heptaplatin.

### SUMMARY

Described herein are methods for treating or preventing a cancer, tumors or other neoplasms in an individual. The methods and compositions of matter comprise combinations of LIF binding polypeptides and a platinum-based antineoplastic agent. These methods may utilize anti-LIF antibodies that antagonize or block LIF activity, and a platinum-based antineoplastic agent. In particular, these combinations exhibit a surprising synergy when compared to either anti-LIF antibodies or platinum-based antineoplastic agent alone.

In one aspect, described herein is the use of a Leukemia Inhibitory Factor (LIF)-binding polypeptide, in combination with a platinum-based antineoplastic agent, for treating a cancer in an individual. In certain embodiments, the LIF-binding polypeptide and the platinum-based antineoplastic agent are administered to the individual in separate formulations. In certain embodiments, the LIF-binding polypeptide and the platinum-based antineoplastic agent are administered to the individual in the same formulation. In certain embodiments, the LIF-binding polypeptide is administered to the individual before the platinum-based antineoplastic agent is administered to the individual. In certain embodiments, the platinum-based antineoplastic agent is administered to the individual before the LIF-binding polypeptide is administered to the individual. In certain embodiments, the platinum-based antineoplastic agent is administered to the individual at the same time as the LIF-binding polypeptide is administered to the individual. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the antibody that specifically binds to LIF comprises at least one framework region derived from a human antibody framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, or 99% identical to the amino acid sequence set forth in SEQ ID NO: 42; and the VL sequence is at least about 80%, 90%, 95%, 97%, 98%, or 99% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the platinum-based antineoplastic agent is cisplatin. In certain embodiments, the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer is refractory to treatment with a therapeutic amount of a LIF-binding polypeptide or a platinum-based antineoplastic agent administered as a monotherapy.

In another aspect, described herein is the use of an antibody that specifically binds Leukemia Inhibitory Factor (LIF), in combination with a platinum-based antineoplastic agent, for treating a cancer in an individual, wherein the LIF binding antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13.

In another aspect, described herein is a method of treating an individual with a cancer comprising administering to the individual with cancer an effective amount of: (a) of a Leukemia Inhibitory Factor (LIF) binding polypeptide; and (b) a platinum-based antineoplastic agent. In certain embodiments, the method comprises administering an effective amount of the LIF-binding polypeptide to the individual with cancer. In certain embodiments, the method comprises administering an effective amount of the platinum-based antineoplastic agent to the individual with cancer. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the antibody that specifically binds to LIF comprises at least one framework region derived from a human antibody framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48 In certain embodiments, the VH sequence is at least about 80%, 90%, 95%, 97%, 98%, 99%, or 100% identical to the amino acid sequence set forth in SEQ ID NO: 42; and the VL sequence is at least about 80%, 90%, 95%, 97%, 98%, 99%, or 100% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, or 99% identical to the amino acid sequence set forth in any one of SEQ ID NOs: 57-60 or 67; and: an immunoglobulin light chain sequence with the amino acid sequence at least about 80%, 90%, 95%, 97%, 98%, or 99% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the platinum-based antineoplastic agent is cisplatin. In certain embodiments, the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer is refractory to treatment with a therapeutic amount of an inhibitor of a LIF-binding polypeptide. In certain embodiments, the cancer is refractory to treatment with a therapeutic amount of a platinum-based antineoplastic agent. In certain embodiments, the Leukemia Inhibitory Factor (LIF) binding polypeptide and the platinum-based antineoplastic agent are administered separately. In certain embodiments, the LIF-binding polypeptide and the platinum-based antineoplastic agent are administered at the same time. In certain embodiments, the LIF-binding polypeptide and the platinum-based antineoplastic agent are administered in a single composition.

In another aspect, described herein is a method of treating an individual with a cancer, the method comprising: administering to the individual with cancer an effective amount of a Leukemia Inhibitory Factor (LIF)-binding polypeptide, wherein the individual has been administered a therapeutic amount of a platinum-based antineoplastic agent. In certain embodiments, the method inhibits growth or metastasis of the cancer. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the LIF-binding polypeptide comprises at least one framework region derived from a human immunoglobulin framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, or 99% identical to the amino acid sequence set forth in SEQ ID NO: 42; and the VL sequence is at least about 80%, 90%, 95%, 97%, 98%, or 99% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the platinum-based antineoplastic agent is cisplatin. In certain embodiments, the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer is refractory to treatment with a therapeutic amount of a platinum-based antineoplastic agent.

In another aspect, described herein is a method of treating an individual with a cancer, the method comprising: administering to the individual with cancer; an effective amount of a platinum-based antineoplastic agent, wherein the individual has been administered a therapeutic amount of a Leukemia Inhibitory Factor (LIF) binding polypeptide. In certain embodiments, the method inhibits growth or metastasis of the cancer. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the LIF-binding polypeptide comprises at least one framework region derived from a human immunoglobulin framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, 99%, or 100% identical to the amino acid sequence set forth in SEQ ID NO: 42; and the VL sequence is at least about 80%, 90%, 95%, 97%, 98%, 99%, or 100% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the platinum-based antineoplastic agent is cisplatin. In certain embodiments, the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer is refractory to treatment with a therapeutic amount of an inhibitor of a LIF-binding polypeptide.

In another aspect, described herein is a method of treating an individual with a cancer comprising administering to the individual with cancer an effective amount of: (a) an antibody that specifically binds Leukemia Inhibitory Factor (LIF) comprising: (i) an immunoglobulin heavy chain complementarity determining region 1 (VH-CDR1) comprising the amino acid sequence set forth in any one of SEQ ID NOs: 1-3; (ii) an immunoglobulin heavy chain complementarity determining region 2 (VH-CDR2) comprising the amino acid sequence set forth in any one of SEQ ID NOs: 4 or 5; (iii) an immunoglobulin heavy chain complementarity determining region 3 (VH-CDR3) comprising the amino acid sequence set forth in any one of SEQ ID NOs: 6-8; (iv) an immunoglobulin light chain complementarity determining region 1 (VL-CDR1) comprising the amino acid sequence set forth in any one of SEQ ID NOs: 9 or 10; (v) an immunoglobulin light chain complementarity determining region 2 (VL-CDR2) comprising the amino acid sequence set forth in any one of SEQ ID NOs: 11 or 12; and (vi) an immunoglobulin light chain complementarity determining region 3 (VL-CDR3) comprising the amino acid sequence set forth in SEQ ID NO: 13; and (b) of a platinum-based antineoplastic agent.

In another aspect, described herein is a kit comprising: (a) a Leukemia Inhibitory Factor (LIF) binding polypeptide; and (b) a platinum-based antineoplastic agent. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the LIF-binding polypeptide comprises at least one framework region derived from a human immunoglobulin framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, 99%, or 100% identical to the amino acid sequence set forth in SEQ ID NO: 42; and the VL sequence is at least about 80%, 90%, 95%, 97%, 98%, 99%, or 100% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the platinum-based antineoplastic agent is cisplatin. In certain embodiments, the kit further comprises a pharmaceutically acceptable excipient, carrier, or diluent.

In another aspect, described herein is a composition comprising: (a) a Leukemia Inhibitory Factor (LIF) binding polypeptide; and (b) a platinum-based antineoplastic agent. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the LIF-binding polypeptide comprises at least one framework region derived from a human immunoglobulin framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, 99%, or 100% identical to the amino acid sequence set forth in SEQ ID NO: 42; and the VL sequence is at least about 80%, 90%, 95%, 97%, 98%, 99%, or 100% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the platinum-based antineoplastic agent is cisplatin. In certain embodiments, the composition further comprises a pharmaceutically acceptable excipient, carrier, or diluent.

### BRIEF DESCRIPTION OF THE DRAWINGS

FIG. 1 depicts a western blot showing inhibition of LIF-induced STAT3 phosphorylation of different anti-LIF humanized antibodies.

FIGS. 2A and 2B depicts a western blot showing inhibition of LIF-induced STAT3 phosphorylation humanized and parental 5D8 antibody.

FIG. 3A shows an IC₅₀ for LIF inhibition in U-251 cells using the h5D8 antibody.

FIG. 3B shows representative IC₅₀ dose response curves of r5D8 and h5D8 inhibition of pSTAT3 under endogenous LIF stimulation conditions. Shown are the representative curves (n=1 h5D8, n=2 r5D8).

FIG. 4 depicts a western blot showing inhibition of LIF-induced STAT3 phosphorylation of different monoclonal antibodies described in this disclosure.

FIG. 5 depicts immunohistochemistry staining and quantitation of LIF expression in glioblastoma multiforme (GBM), NSCLC (non-small cell lung carcinoma), ovarian cancer, colorectal cancer, and pancreatic tumors from human patients. Bars represent mean+/−standard error of the mean (SEM).

FIG. 6A is a graph showing an experiment conducted in a mouse model of non-small cell lung cancer using the humanized 5D8 antibody.

FIG. 6B is a graph showing an experiment conducted in a mouse model of non-small cell lung cancer using the r5D8 antibody.

FIG. 7A shows the effect of r5D8 on inhibition of U251 cells in an orthotopic mouse model of GBM. Quantitation shown at day 26.

FIG. 7B shows data from mice inoculated with luciferase expressing human U251 GBM cells and then treated with 100, 200 or 300 μg of h5D8 or vehicle twice a week. Tumor size was determined by bioluminescence (Xenogen IVIS Spectrum) on day 7. The graph shows individual tumor measurements with horizontal bars indicating mean±SEM. Statistical significance was calculated using the unpaired non-parametric Mann-Whitney U-test.

FIG. 8A shows the effect of r5D8 on inhibition of growth of ovarian cancer cells in an syngeneic mouse model.

FIG. 8B shows the individual measurements of tumors at day 25.

FIG. 8C illustrates that h5D8 shows a significant reduction in tumor growth when administered at 200 μg/mouse twice weekly (p<0.05). Symbols are mean+SEM, statistical significance compared with vehicle (with unpaired non-parametric Mann-Whitney U-test).

FIG. 9A shows the effect of r5D8 on inhibition of growth of colorectal cancer cells in a syngeneic mouse model.

FIG. 9B shows the individual measurements of tumors at day 17.

FIG. 10A shows reduction of macrophage infiltration to tumor sites in an orthotopic mouse model of GBM with a representative image and quantitation of CCL22+ cells.

FIG. 10B shows reduction of macrophage infiltration in a human organotypic tissue slice culture model.

FIG. 10C shows reduction of macrophage infiltration to tumor sites in a syngeneic mouse model of ovarian cancer with a representative image and quantitation of CCL22+ cells.

FIG. 10D shows reduction of macrophage infiltration to tumor sites in a syngeneic mouse model of colorectal cancer with a representative image and quantitation of CCL22+ cells.

FIG. 10E shows the inflammatory phenotype of tumor associated macrophages (TAMs) harvested from tumors treated with h5D8 (15 mg/kg, 2 QW) on day 25 (endpoint). TAMs in treated tumors were polarized towards the M1 pro-inflammatory phenotype. Statistical significance was determined by an unpaired t-test.

FIG. 10F shows gene expression data of monocytes cultured with the conditioned media of LIF-knockdown cells.

FIG. 11A shows increases in non-myeloid effector cells in a syngeneic mouse model of ovarian cancer after treatment with r5D8.

FIG. 11B shows increases in non-myeloid effector cells in a syngeneic mouse model of colorectal cancer after treatment with r5D8.

FIG. 11C shows decreases in percentage of CD4+ T_{REG} cells in a mouse model of NSCLC cancer after treatment with r5D8.

FIG. 12 shows data from mice bearing CT26 tumors treated twice weekly with PBS (control) or r5D8 administered intraperitoneally in the presence or absence of anti-CD4 and anti-CD8 depleting antibodies. The graph shows individual tumor measurements at d13 expressed as mean tumor volume+SEM. Statistical differences between groups was determined by unpaired non-parametric Mann-Whitney U-test. R5D8 inhibited the growth of CT26 tumors (*p<0.05). The tumor growth inhibition by r5D8 was significantly reduced in the presence of anti-CD4 and anti-CD8 depleting antibodies (****p<0.0001).

FIG. 13A illustrates an overview of the co-crystal structure of h5D8 Fab in complex with LIF. The gp130 interacting site is mapped on the surface of LIF (dark shaded).

FIG. 13B illustrates detailed interactions between LIF and h5D8, showing residues forming salt bridges and h5D8 residues with buried surface areas greater than 100 Å².

FIG. 14A illustrates superposition of the five h5D8 Fab crystal structures and indicates a high degree of similarity despite being crystallized in different chemical conditions.

FIG. 14B illustrates an extensive network of Van der Waals interactions mediated by unpaired Cys100. This residue is well-ordered, partakes in shaping the conformations of HCDR1 and HCDR3 and is not involved in undesired disulfide scrambling. Distances between residues are shown as dashed lines and labeled.

FIG. 15A illustrates binding of h5D8 C100 mutants to human LIF by ELISA.

FIG. 15B illustrates binding of h5D8 C100 mutants to mouse LIF by ELISA.

FIG. 16A illustrates that h5D8 does not block binding between LIF and LIFR by Octet. Sequential binding of h5D8 to LIF followed by LIFR.

FIGS. 16B and 16C illustrate ELISA analysis of LIF/mAb complexes binding to immobilized LIFR or gp130. Signals of species-specific peroxidase conjugated anti-IgG antibodies (anti-human for (−) and h5D8, anti-rat for r5d8 and B09) detecting the antibody portion of mAb/LIF complexes binding immobilized LIFR (FIG. 16B) or gp130 (FIG. 16C) coated plates.

FIGS. 17A and 17B illustrate mRNA expression of LIF (FIG. 17A) or LIFR (FIG. 18B) in 72 different human tissues.

FIG. 18 shows data from mice with subcutaneously implanted CT26 tumors that were treated with h5D8 and cisplatin. This figure shows a time course of tumor growth from averaged tumor volumes for vehicle, control IgG1, h5D8 alone, cisplatin alone, and h5D8+cisplatin combination groups.

FIGS. 19A and 19B show tumor volume data from mice with subcutaneously implanted CT26 tumors that were treated with h5D8 and doxorubicin (FIG. 19A) or with h5D8 and paclitaxel (FIG. 19B) over time.

FIG. 20 shows data from mice with intradermally implanted CT26 tumors that were treated with h5D8 and cisplatin. This figure shows a time course of tumor growth from averaged tumor volumes for vehicle, control IgG1, h5D8 alone, cisplatin alone, and h5D8+cisplatin combination groups.

### DETAILED DESCRIPTION

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

In one aspect, described herein is the use of a Leukemia Inhibitory Factor (LIF)-binding polypeptide, in combination with a platinum-based antineoplastic agent, for treating a cancer in an individual.

In another aspect, described herein is the use of a Leukemia Inhibitory Factor (LIF)-binding antibody, in combination with a platinum-based antineoplastic agent, for treating a cancer in an individual, wherein the LIF binding antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13.

In another aspect, described herein is a method of treating an individual with a cancer comprising administering to the individual with cancer an effective amount of: (a) a Leukemia Inhibitory Factor (LIF) binding polypeptide; and (b) a platinum-based antineoplastic agent.

In another aspect, described herein is a method of treating an individual with a cancer, the method comprising: administering to the individual with cancer an effective amount of a Leukemia Inhibitory Factor (LIF)-binding polypeptide, wherein the individual has been administered a therapeutic amount of a platinum-based antineoplastic agent.

In another aspect, described herein is a method of treating an individual with a cancer, the method comprising: administering to the individual with cancer; an effective amount of a platinum-based antineoplastic agent, wherein the individual has been administered a therapeutic amount of a Leukemia Inhibitory Factor (LIF) binding polypeptide.

In another aspect, described herein is a method of treating an individual with a cancer comprising administering to the individual with cancer an effective amount of: (a) a Leukemia Inhibitory Factor (LIF) binding antibody comprising: (i) an immunoglobulin heavy chain complementarity determining region 1 (VH-CDR1) comprising the amino acid sequence set forth in any one of SEQ ID NOs: 1-3; (ii) an immunoglobulin heavy chain complementarity determining region 2 (VH-CDR2) comprising the amino acid sequence set forth in any one of SEQ ID NOs: 4 or 5; (iii) an immunoglobulin heavy chain complementarity determining region 3 (VH-CDR3) comprising the amino acid sequence set forth in any one of SEQ ID NOs: 6-8; (iv) an immunoglobulin light chain complementarity determining region 1 (VL-CDR1) comprising the amino acid sequence set forth in any one of SEQ ID NOs: 9 or 10; (v) an immunoglobulin light chain complementarity determining region 2 (VL-CDR2) comprising the amino acid sequence set forth in any one of SEQ ID NOs: 11 or 12; and (vi) an immunoglobulin light chain complementarity determining region 3 (VL-CDR3) comprising the amino acid sequence set forth in SEQ ID NO: 13; and (b) a platinum-based antineoplastic agent.

In another aspect, described herein is a kit comprising: (a) a Leukemia Inhibitory Factor (LIF) binding polypeptide; and (b) a platinum-based antineoplastic agent.

In another aspect, described herein is a composition comprising: (a) a Leukemia Inhibitory Factor (LIF) binding polypeptide; and (b) a platinum-based antineoplastic agent.

As used herein the terms “individual,” “subject,” and “patient” are used interchangeably and include humans diagnosed with or suspected of being afflicted with a tumor, a cancer, or other neoplasm.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

As used herein the term an “effective amount” refers to the amount of a therapeutic that causes a biological effect when administered to a mammal. Biological effects include, but are not limited to, inhibition or blockade a receptor ligand interaction (e.g., LIF-LIFR), inhibition of a signaling pathway (e.g., STAT3 phosphorylation), reduced tumor growth, reduced tumor metastasis, or prolonged survival of an animal bearing a tumor. A “therapeutic amount” is the concertation of a drug calculated to exert a therapeutic effect. A therapeutic amount encompasses the range of dosages capable of inducing a therapeutic response in a population of individuals. The mammal can be a human individual. The human individual can be afflicted with or suspected or being afflicted with a tumor.

As used herein, unless otherwise indicated, the term “antibody” includes antigen binding fragments of antibodies, i.e. antibody fragments that retain the ability to bind specifically to the antigen bound by the full-length antibody, e.g. fragments that retain one or more CDR regions. Examples of antibody fragments include, but are not limited to, Fab, Fab′, F(ab′)2, and Fv fragments; diabodies; linear antibodies; heavy chain antibodies, single-chain antibody molecules, e.g. single-chain variable region fragments (scFv), nanobodies and multispecific antibodies formed from antibody fragments with separate specificities, such as a bispecific antibody. In certain embodiments, the antibodies are humanized in such a way as to reduce an individual's immune response to the antibody. For example, the antibodies may be chimeric, e.g. non-human variable region with human constant region, or CDR grafted, e.g. non-human CDR regions with human constant region and variable region framework sequences. In certain embodiments, antibodies are deimmunized after humanization. Deimmunization involves removing or mutating one or more T-cell epitopes in the constant region of the antibody. In certain embodiments, the antibodies described herein are monoclonal. As used herein a “recombinant antibody” is an antibody that comprises an amino acid sequence derived from two different species or, or two different sources, and includes synthetic molecules, for example, an antibody that comprises a non-human CDR and a human framework or constant region. In certain embodiments, recombinant antibodies of the present invention are produced from a recombinant DNA molecule or synthesized.

Percent (%) sequence identity with respect to a reference polypeptide or antibody sequence is the percentage of amino acid residues in a candidate sequence that are identical with the amino acid residues in the reference polypeptide or antibody sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN, or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as saccharides or lipids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules.

The term “antineoplastic agent” refers to agents capable of acting to prevent, inhibit, or halt the development of a cancer a tumor or a neoplasm. Antineoplastic agents may be also known as chemotherapeutic agents. An antineoplastic agent may target and destroy cancer cells. Antineoplastic agents may include, but are not limited to, antimetabolites, biological response modifiers, bleomycins, DNA alkylating agents, DNA cross-linking agents, enzymes, hormones, monoclonal antibodies, platinum complexes, proteasome inhibitors, taxanes, vincas, topoisomerase inhibitors, tyrosine kinase inhibitors, nucleoside analogues, antifolates, anthracyclines, podophyllotoxins, alkylating agents, mTOR inhibitors, retinoids, histone deacetylase inhibitors, and immunomodulatory agents.

### Structural Attributes of the Antibodies Described Herein

A complementarity determining region (“CDR”) is a part of an immunoglobulin (antibody) variable region that is primarily responsible for the antigen binding specificity of the antibody. CDR regions are highly variable from one antibody to the next even when the antibody specifically binds the same target or epitope. A heavy chain variable region comprises three CDR regions, abbreviated VH-CDR1, VH-CDR2, and VH-CDR3; and a light chain variable region comprises three CDR regions, abbreviated VL-CDR1, VL-CDR2, and VL-CDR3. These CDR regions are ordered consecutively in the variable region with the CDR1 being the most N-terminal and the CDR3 being the most C-terminal. Interspersed between the CDRs are framework regions which contribute to the structure and display much less variability than the CDR regions. A heavy chain variable region comprises four framework regions, abbreviated VH-FR1, VH-FR2, VH-FR3, and VH-FR4; and a light chain variable region comprises four framework regions, abbreviated VL-FR1, VL-FR2, VL-FR3, and VL-FR4. Complete full-sized bivalent antibodies comprising two heavy and light chains will comprise: 12 CDRs, with three unique heavy chain CDRs and three unique light chain CDRs; 16 FR regions, with four unique heavy chain FR regions and four unique light chain FR regions. In certain embodiments, the antibodies described herein minimally comprise three heavy chain CDRs. In certain embodiments, the antibodies described herein minimally comprise three light chain CDRs. In certain embodiments, the antibodies described herein minimally comprise three heavy chain CDRs and three light chain CDRs. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” (“Contact” numbering scheme); Lefranc M P et al.,“IMGT unique numbering for immunoglobulin and T cell receptor variable domains and Ig superfamily V-like domains,” Dev Comp Immunol, 2003 January; 27(1):55-77 (“IMGT” numbering scheme); and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme). CDRs are identified herein from variable sequences provided using different numbering systems, herein with the Kabat, the IMGT, the Chothia numbering system, or any combination of the three. The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)). In certain embodiments, the antibodies described herein are humanized. In certain embodiments, the antibodies described herein are chimeric. In certain embodiments, the antibodies described herein comprise variable regions of rat origin. In certain embodiments, the antibodies described herein comprise CDRs of rat origin. In certain embodiments, the antibodies described herein comprise variable regions of mouse origin. In certain embodiments, the antibodies described herein comprise CDRs of mouse origin.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex is analyzed to identify contact points between the antibody and antigen. Such contact residues and neighboring residues may be targeted or eliminated as candidates for substitution. Variants may be screened to determine whether they contain the desired properties.

In certain embodiments, the antibodies described herein comprise a constant region in addition to a variable region. The heavy chain constant region (C_{H}) comprises four domains abbreviated C_{H}1, C_{H}2, C_{H}3, and C_{H}4, located at the C-terminal end of the full heavy chain polypeptide, C-terminal to the variable region. The light chain constant region (C_{L}) is much smaller than the C_{H} and is located at the C-terminal end of the full light chain polypeptide, C-terminal to the variable region. The constant region is highly conserved and comprises different isotypes that are associated with slightly different functions and properties. In certain embodiments, the constant region is dispensable for antibody binding to a target antigen. In certain embodiments, the constant regions of the antibody, both heavy and light chains are dispensable for antibody binding. In certain embodiments, the antibodies described herein lack one or more of a light chain constant region, heavy chain constant region, or both. Most monoclonal antibodies are of an IgG isotype; which is further divided into four subclasses IgG₁, IgG₂, IgG₃, and IgG₄. In certain embodiments, the antibodies described herein comprise any IgG subclass. In certain embodiments, the IgG subclass comprises IgG₁. In certain embodiments, the IgG subclass comprises IgG₂. In certain embodiments, the IgG subclass comprises IgG₃. In certain embodiments, the IgG subclass comprises IgG₄.

Antibodies comprise a fragment crystallizable region (Fc region) that is responsible for binding to complement and Fc receptors. The Fc region comprises the C_{H}2, C_{H}3, and C_{H}4 regions of the antibody molecule. The Fc region of an antibody is responsible for activating complement and antibody dependent cell cytotoxicity (ADCC). The Fc region also contributes to an antibody's serum half-life. In certain embodiments, the Fc region of the antibodies described herein comprises one or more amino acid substitutions that promote complement mediated cell lysis. In certain embodiments, the Fc region of antibodies described herein comprises one or more amino acid substitutions that promote ADCC. In certain embodiments, the Fc region of antibodies described herein comprises one or more amino acid substitutions that reduce complement mediated cell lysis. In certain embodiments, the Fc region of antibodies described herein comprises one or more amino acid substitutions that increase binding of the antibody to an Fc receptor. In certain embodiments, the Fc receptor comprises FcγRI (CD64), FcγRIIA (CD32), FcγRIIIA (CD16a), FcγRIIIB (CD16b), or any combination thereof. In certain embodiments, the Fc region of the antibodies described herein comprises one or more amino acid substitutions that increase the serum half-life of the antibody. In certain embodiments, the one or more amino acid substitutions that increase the serum half-life of the antibody increase affinity of the antibody to the neonatal Fc receptor (FcRn).

In some embodiments, the antibodies of this disclosure are variants that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest are described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

Antibodies useful in the clinic are often “humanized” to reduce immunogenicity in human individuals. Humanized antibodies improve safety and efficacy of monoclonal antibody therapy. One common method of humanization is to produce a monoclonal antibody in any suitable animal (e.g., mouse, rat, hamster) and replace the constant region with a human constant region, antibodies engineered in this way are termed “chimeric”. Another common method is “CDR grafting” which replaces the non-human V-FRs with human V-FRs. In the CDR grafting method all residues except for the CDR region are of human origin. In certain embodiments, the antibodies described herein are humanized. In certain embodiments, the antibodies described herein are chimeric. In certain embodiments, the antibodies described herein are CDR grafted.

Humanization generally reduces or has little effect on the overall affinity of the antibody. Described herein are antibodies that unexpectedly possess greater affinity for their target after humanization. In certain embodiments, humanization increases the affinity for the antibody by 10%. In certain embodiments, humanization increases the affinity for the antibody by 25%. In certain embodiments, humanization increases the affinity for the antibody by 35%. In certain embodiments, humanization increases the affinity for the antibody by 50%. In certain embodiments, humanization increases the affinity for the antibody by 60%. In certain embodiments, humanization increases the affinity for the antibody by 75%. In certain embodiments, humanization increases the affinity for the antibody by 100%. Affinity is suitably measured using surface plasmon resonance (SPR). In certain embodiments, affinity is measured using glycosylated human LIF. In certain embodiments, the glycosylated human LIF is immobilized to the surface of the SPR chip. In certain embodiments, the antibody binds with a K_{D} of less than about 300 nanomolar, 200 nanomolar,150 nanomolar, 125 nanomolar 100 nanomolar, 90 nanomolar, 80 nanomolar, 70 nanomolar, 60 nanomolar, 50 nanomolar, 40 nanomolar, or less.

The compositions and methods described herein comprise combinations of LIF-binding polypeptides with platinum-based antineoplastic agents. In certain embodiments, the LIF-binding polypeptide comprises a fragment of an immunoglobulin variable region, or an immunoglobulin heavy chain constant region. In certain embodiments, the LIF-binding polypeptide is an antibody that specifically binds to LIF. In certain embodiments, the LIF-binding antibody comprises at least one framework region derived from a human immunoglobulin framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody is the h5D8 antibody described herein.

In certain embodiments, described herein the antibody utilized or administered in combination with a platinum-based antineoplastic agent is the h5D8 antibody. The h5d8 antibody specifically binds LIF comprises a VH-CDR1 set forth in any one of SEQ ID NOs: 1-3, a VH-CDR2 set forth in any one of SEQ ID NOs: 4 or 5, and a VH-CDR3 set forth in any one of SEQ ID NOs: 6 to 8. In certain embodiments, described herein, h5D8 specifically binds LIF and comprises a VL-CDR1 set forth in any one of SEQ ID NOs: 9 or 10, a VL-CDR2 set forth in SEQ ID NOs: 11 or 12, and a VL-CDR3 set forth in SEQ ID NO: 13. In certain embodiments, described herein, h5D8 specifically binds LIF and comprises a VH-CDR1 set forth in any one of SEQ ID NOs: 1-3, a VH-CDR2 set forth in any one of SEQ ID NOs: 4 or 5, and a VH-CDR3 set forth in any one of SEQ ID NOs: 6-8, a VL-CDR1 set forth in any one of SEQ ID NOs: 9 or 10, a VL-CDR2 set forth in SEQ ID NOs: 11 or 12, and a VL-CDR3 set forth in SEQ ID NO: 13. The VH and VL regions of h5D8 are set forth by SEQ ID NOs: 42 and 46.

In certain embodiments, the antibody that specifically binds LIF comprises one or more human heavy chain framework regions comprising: a VH-FR1 amino acid sequence at least about 80%, 90%, 95%, 97%, 98%, or 99% identical to the amino acid sequence set forth in any one of SEQ ID NOs: 14-17, a VH-FR2 amino acid sequence at least about 80%, 90%, 95%, 97%, 98%, or 99% identical to the amino acid sequence set forth in any one of SEQ ID NOs: 18 or 19, a VH-FR3 amino acid sequence at least about 80%, 90%, 95%, 97%, 98%, or 99% identical to the amino acid sequence set forth in any one of SEQ ID NOs: 20-22, or a VH-FR4 region amino acid sequence at least about 80%, 90%, 95%, 97%, 98%, or 99% identical to the amino acid sequence set forth in any one of SEQ ID NOs: 23-25. In certain embodiments, the one or more human heavy chain framework regions comprise a VH-FR1 amino acid sequence at least about 80%, 90%, 95%, 97%, 98%, or 99% identical to the amino acid sequence set forth in SEQ ID NO: 15, a VH-FR2 amino acid sequence at least about 80%, 90%, 95%, 97%, 98%, or 99% identical to the amino acid sequence set forth in SEQ ID NO: 19, a VH-FR3 amino acid sequence at least about 80%, 90%, 95%, 97%, 98%, or 99% identical to the amino acid sequence set forth in SEQ ID NO: 20, and a VH-FR4 amino acid sequence at least about 80%, 90%, 95%, 97%, 98%, or 99% identical to the amino acid sequence set forth in SEQ ID NO: 24. In certain embodiments, the antibody that specifically binds LIF comprises one or more human light chain framework regions comprising: a VL-FR1 amino acid sequence at least about 80%, 90%, 95%, 97%, 98%, or 99% identical to the amino acid sequence set forth in any one of SEQ ID NOs: 26-29, a VL-FR2 amino acid sequence at least about 80%, 90%, 95%, 97%, 98%, or 99% identical to the amino acid sequence set forth in any one of SEQ ID NOs: 30-33, a VL-FR3 amino acid sequence at least about 80%, 90%, 95%, 97%, 98%, or 99% identical to the amino acid sequence set forth in any one of SEQ ID NOs: 34-37, or a VL-FR4 amino acid sequence at least about 80%, 90%, 95%, 97%, 98%, or 99% identical to the amino acid sequence set forth in any one of SEQ ID NOs: 38-40. In certain embodiments, the one or more human light chain framework regions comprise a VL-FR1 amino acid sequence at least about 80%, about 90%, or about 95% identical to the amino acid sequence set forth in SEQ ID NO: 27, a VL-FR2 amino acid sequence at least about 80%, 90%, 95%, 97%, 98%, or 99% identical to the amino acid sequence set forth in SEQ ID NO: 31, a VL-FR3 amino acid sequence at least about 80%, 90%, 95%, 97%, 98%, or 99% identical to the amino acid sequence set forth in SEQ ID NO: 35, and a VL-FR4 amino acid sequence at least about 80%, 90%, 95%, 97%, 98%, or 99% identical to the amino acid sequence set forth in SEQ ID NO: 38. In certain embodiments, the one or more human heavy chain framework regions and the one or more human light chain regions comprise a VH-FR1 amino acid sequence at least about 80%, 90%, 95%, 97%, 98%, or 99% identical to the amino acid sequence set forth in SEQ ID NO: 15, a VH-FR2 amino acid sequence at least about 80%, 90%, 95%, 97%, 98%, or 99% identical to the amino acid sequence set forth in SEQ ID NO: 19, a VH-FR3 amino acid sequence at least about 80%, 90%, 95%, 97%, 98%, or 99% identical to the amino acid sequence set forth in SEQ ID NO: 20, a VH-FR4 amino acid sequence at least about 80%, 90%, 95%, 97%, 98%, or 99% identical to the amino acid sequence set forth in SEQ ID NO: 24, a VL-FR1 amino acid sequence at least about 80%, 90%, 95%, 97%, 98%, or 99% identical to the amino acid sequence set forth in SEQ ID NO: 27, a VL-FR2 amino acid sequence at least about 80%, 90%, 95%, 97%, 98%, or 99% identical to the amino acid sequence set forth in SEQ ID NO: 31, a VL-FR3 amino acid sequence at least about 80%, 90%, 95%, 97%, 98%, or 99% identical to the amino acid sequence set forth in SEQ ID NO: 35, and a VL-FR4 amino acid sequence at least 80%, 90%, 95%, 97%, 98%, or 99% identical to the amino acid sequence set forth in SEQ ID NO: 38. In certain embodiments, the antibody that specifically binds LIF comprises one or more human heavy chain framework regions comprising: a VH-FR1 amino acid sequence identical to the amino acid sequence set forth in any one of SEQ ID NOs: 14-17, a VH-FR2 amino acid sequence identical to the amino acid sequence set forth in any one of SEQ ID NOs: 18 or 19, a VH-FR3 amino acid sequence identical to the amino acid sequence set forth in any one of SEQ ID NOs: 20-22, or a VH-FR4 region amino acid sequence identical to the amino acid sequence set forth in any one of SEQ ID NOs: 23-25. In certain embodiments, the one or more human heavy chain framework regions comprise a VH-FR1 amino acid sequence identical to the amino acid sequence set forth in SEQ ID NO: 15, a VH-FR2 amino acid sequence identical to the amino acid sequence set forth in SEQ ID NO: 19, a VH-FR3 amino acid sequence identical to the amino acid sequence set forth in SEQ ID NO: 20, and a VH-FR4 amino acid sequence identical to the amino acid sequence set forth in SEQ ID NO: 24. In certain embodiments, the antibody that specifically binds LIF comprises one or more human light chain framework regions comprising: a VL-FR1 amino acid sequence identical to the amino acid sequence set forth in any one of SEQ ID NOs: 26-29, a VL-FR2 amino acid sequence identical to the amino acid sequence set forth in any one of SEQ ID NOs: 30-33, a VL-FR3 amino acid sequence identical to the amino acid sequence set forth in any one of SEQ ID NOs: 34-37, or a VL-FR4 amino acid sequence identical to the amino acid sequence set forth in any one of SEQ ID NOs: 38-40. In certain embodiments, the one or more human light chain framework regions comprise a VL-FR1 amino acid sequence identical to the amino acid sequence set forth in SEQ ID NO: 27, a VL-FR2 amino acid sequence identical to the amino acid sequence set forth in SEQ ID NO: 31, a VL-FR3 amino acid sequence identical to the amino acid sequence set forth in SEQ ID NO: 35, and a VL-FR4 amino acid sequence identical to the amino acid sequence set forth in SEQ ID NO: 38. In certain embodiments, the one or more human heavy chain framework regions and the one or more human light chain regions comprise a VH-FR1 amino acid sequence identical to the amino acid sequence set forth in SEQ ID NO: 15, a VH-FR2 amino acid sequence identical to the amino acid sequence set forth in SEQ ID NO: 19, a VH-FR3 amino acid sequence identical to the amino acid sequence set forth in SEQ ID NO: 20, a VH-FR4 amino acid sequence identical to the amino acid sequence set forth in SEQ ID NO: 24, a VL-FR1 amino acid sequence identical to the amino acid sequence set forth in SEQ ID NO: 27, a VL-FR2 amino acid sequence identical to the amino acid sequence set forth in SEQ ID NO: 31, a VL-FR3 amino acid sequence identical to the amino acid sequence set forth in SEQ ID NO: 35, and a VL-FR4 amino acid sequence identical to the amino acid sequence set forth in SEQ ID NO: 38. In certain embodiments, the antibody specifically binds human LIF.

The r5D8 antibody described herein was generated from rats immunized with DNA encoding human LIF. r5D8 was cloned and sequenced and comprises CDRs (using the combination of the Kabat and IMGT CDR numbering methods) with the following amino acid sequences: a VH-CDR1 corresponding to SEQ ID NO: 1 (GFTFSHAWMH), a VH-CDR2 corresponding to SEQ ID NO: 4 (QIKAKSDDYATYYAESVKG), a VH-CDR3 corresponding to SEQ ID NO: 6 (TCWEWDLDF), a VL-CDR1 corresponding to SEQ ID NO: 9 (RSSQSLLDSDGHTYLN), a VL-CDR2 corresponding to SEQ ID NO: 11 (SVSNLES), and a VL-CDR3 corresponding to SEQ ID NO: 13 (MQATHAPPYT). This antibody has been humanized by CDR grafting and the humanized version is referred to as h5D8.

In certain embodiments, described herein is an antibody that specifically binds LIF comprising a VH-CDR1at least 80% or 90% identical to that set forth in SEQ ID NO: 1 (GFTFSHAWMH), a VH-CDR2 at least 80%, 90%, or 95% identical to that set forth in SEQ ID NO: 4 (QIKAKSDDYATYYAESVKG), and a VH-CDR3 at least 80% or 90% identical to that set forth in SEQ ID NO: 6 (TCWEWDLDF). In certain embodiments, described herein is an antibody that specifically binds LIF comprising a VL-CDR1 at least 80% or 90% identical to that set forth in SEQ ID NO: 9 (RSSQSLLDSDGHTYLN), a VL-CDR2 at least 80% identical to that set forth in SEQ ID NO: 11 (SVSNLES), and a VL-CDR3 at least 80% or 90% identical to that set forth in SEQ ID NO: 13 (MQATHAPPYT). In certain embodiments, described herein is an antibody that specifically binds LIF comprising a VH-CDR1 set forth in SEQ ID NO: 1 (GFTFSHAWMH), a VH-CDR2 set forth in SEQ ID NO: 4 (QIKAKSDDYATYYAESVKG), a VH-CDR3 set forth in SEQ ID NO: 6 (TCWEWDLDF), a VL-CDR1 set forth in SEQ ID NO: 9 (RSSQSLLDSDGHTYLN), a VL-CDR2 set forth in SEQ ID NO: 11 (SVSNLES), and a VL-CDR3 set forth in SEQ ID NO: 13 (MQATHAPPYT). Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 1, 4, 6, 9, 11, and 13 by 1, 2, 3, or 4 amino acids. In certain embodiments, the antibody comprises CDRs that differ from the amino acid sequence set forth in any one of SEQ ID NOs: 1, 4, 6, 9, 11, and 13 by 1, 2, 3, or 4 amino acids and does not affect the binding affinity by greater than 10%, 20%, or 30%. In certain embodiments, antibodies that specifically bind LIF comprise one or more human heavy chain framework regions comprising: a VH-FR1 amino acid sequence at least about 80%, 90%, 95%, 97%, 98%, or 99% identical to the amino acid sequence set forth in any one of SEQ ID NOs: 14-17, a VH-FR2 amino acid sequence at least about 80%, 90%, 95%, 97%, 98%, or 99% identical to the amino acid sequence set forth in any one of SEQ ID NOs: 18 or 19, a VH-FR3 amino acid sequence at least 90% identical to the amino acid sequence set forth in any one of SEQ ID NOs: 20-22, or a VH-FR4 region amino acid sequence at least about 80%, 90%, 95%, 97%, 98%, or 99% identical to the amino acid sequence set forth in any one of SEQ ID NOs: 23-25. In certain embodiments, the one or more human heavy chain framework regions comprises a VH-FR1 amino acid sequence at least about 80%, 90%, 95%, 97%, 98%, or 99% identical to the amino acid sequence set forth in SEQ ID NO: 15, a VH-FR2 amino acid sequence at least about 80%, 90%, 95%, 97%, 98%, or 99% identical to the amino acid sequence set forth in SEQ ID NO: 19, a VH-FR3 amino acid sequence at least about 80%, 90%, 95%, 97%, 98%, or 99% identical to the amino acid sequence set forth in SEQ ID NO: 20, and a VH-FR4 amino acid sequence at least about 80%, 90%, 95%, 97%, 98%, or 99% identical to the amino acid sequence set forth in SEQ ID NO: 24. In certain embodiments, the antibody that specifically binds LIF comprises one or more human light chain framework regions comprising: a VL-FR1 amino acid sequence at least about 80%, 90%, 95%, 97%, 98%, or 99% identical to the amino acid sequence set forth in any one of SEQ ID NOs: 26-29, a VL-FR2 amino acid sequence at least about 80%, 90%, 95%, 97%, 98%, or 99% identical to the amino acid sequence set forth in any one of SEQ ID NOs: 30-33, a VL-FR3 amino acid sequence at least about 80%, 90%, 95%, 97%, 98%, or 99% identical to the amino acid sequence set forth in any one of SEQ ID NOs: 34-37, or a VL-FR4 amino acid sequence at least about 80%, 90%, 95%, 97%, 98%, or 99% identical to the amino acid sequence set forth in any one of SEQ ID NOs: 38-40. In certain embodiments, the one or more human light chain framework regions comprise a VL-FR1 amino acid sequence at least about 80%, 90%, 95%, 97%, 98%, or 99% identical to the amino acid sequence set forth in SEQ ID NO: 27, a VL-FR2 amino acid sequence at least about 80%, 90%, 95%, 97%, 98%, or 99% identical to the amino acid sequence set forth in SEQ ID NO: 31, a VL-FR3 amino acid sequence at least about 80%, 90%, 95%, 97%, 98%, or 99% identical to the amino acid sequence set forth in SEQ ID NO: 35, and a VL-FR4 amino acid sequence at least about 80%, 90%, 95%, 97%, 98%, or 99% % identical to the amino acid sequence set forth in SEQ ID NO: 38. In certain embodiments, the one or more human heavy chain framework regions and the one or more human light chain regions comprise all of a VH-FR1 amino acid sequence at least about 80%, 90%, 95%, 97%, 98%, or 99% identical to the amino acid sequence set forth in SEQ ID NO: 15, a VH-FR2 amino acid sequence at least about 80%, 90%, 95%, 97%, 98%, or 99% identical to the amino acid sequence set forth in SEQ ID NO: 19, a VH-FR3 amino acid sequence at least about 80%, 90%, 95%, 97%, 98%, or 99% identical to the amino acid sequence set forth in SEQ ID NO: 20, a VH-FR4 amino acid sequence at least 90% identical to the amino acid sequence set forth in SEQ ID NO: 24, a VL-FR1 amino acid sequence at least about 80%, 90%, 95%, 97%, 98%, or 99% identical to the amino acid sequence set forth in SEQ ID NO: 27, a VL-FR2 amino acid sequence at least about 80%, 90%, 95%, 97%, 98%, or 99% identical to the amino acid sequence set forth in SEQ ID NO: 31, a VL-FR3 amino acid sequence at least about 80%, 90%, 95%, 97%, 98%, or 99% identical to the amino acid sequence set forth in SEQ ID NO: 35, and a VL-FR4 amino acid sequence at least about 80%, 90%, 95%, 97%, 98%, or 99% identical to the amino acid sequence set forth in SEQ ID NO: 38. In certain embodiments, the antibody specifically binds human LIF. In certain embodiments, the antibody that specifically binds LIF comprises one or more human heavy chain framework regions comprising: a VH-FR1 amino acid sequence identical to the amino acid sequence set forth in any one of SEQ ID NOs: 14-17, a VH-FR2 amino acid sequence identical to the amino acid sequence set forth in any one of SEQ ID NOs: 18 or 19, a VH-FR3 amino acid sequence identical to the amino acid sequence set forth in any one of SEQ ID NOs: 20-22, or a VH-FR4 region amino acid sequence identical to the amino acid sequence set forth in any one of SEQ ID NOs: 23-25. In certain embodiments, the one or more human heavy chain framework regions comprise a VH-FR1 amino acid sequence identical to the amino acid sequence set forth in SEQ ID NO: 15, a VH-FR2 amino acid sequence identical to the amino acid sequence set forth in SEQ ID NO: 19, a VH-FR3 amino acid sequence identical to the amino acid sequence set forth in SEQ ID NO: 20, and a VH-FR4 amino acid sequence identical to the amino acid sequence set forth in SEQ ID NO: 24. In certain embodiments, the antibody that specifically binds LIF comprises one or more human light chain framework regions comprising: a VL-FR1 amino acid sequence identical to the amino acid sequence set forth in any one of SEQ ID NOs: 26-29, a VL-FR2 amino acid sequence identical to the amino acid sequence set forth in any one of SEQ ID NOs: 30-33, a VL-FR3 amino acid sequence identical to the amino acid sequence set forth in any one of SEQ ID NOs: 34-37, or a VL-FR4 amino acid sequence identical to the amino acid sequence set forth in any one of SEQ ID NOs: 38-40. In certain embodiments, the one or more human light chain framework regions comprise a VL-FR1 amino acid sequence identical to the amino acid sequence set forth in SEQ ID NO: 27, a VL-FR2 amino acid sequence identical to the amino acid sequence set forth in SEQ ID NO: 31, a VL-FR3 amino acid sequence identical to the amino acid sequence set forth in SEQ ID NO: 35, and a VL-FR4 amino acid sequence identical to the amino acid sequence set forth in SEQ ID NO: 38. In certain embodiments, the one or more human heavy chain framework regions and the one or more human light chain regions comprise a VH-FR1 amino acid sequence identical to the amino acid sequence set forth in SEQ ID NO: 15, a VH-FR2 amino acid sequence identical to the amino acid sequence set forth in SEQ ID NO: 19, a VH-FR3 amino acid sequence identical to the amino acid sequence set forth in SEQ ID NO: 20, a VH-FR4 amino acid sequence identical to the amino acid sequence set forth in SEQ ID NO: 24, a VL-FR1 amino acid sequence identical to the amino acid sequence set forth in SEQ ID NO: 27, a VL-FR2 amino acid sequence identical to the amino acid sequence set forth in SEQ ID NO: 31, a VL-FR3 amino acid sequence identical to the amino acid sequence set forth in SEQ ID NO: 35, and a VL-FR4 amino acid sequence identical to the amino acid sequence set forth in SEQ ID NO: 38. In certain embodiments, the antibody specifically binds human LIF.

In certain embodiments, described herein is an antibody that specifically binds LIF comprising a VH-CDR1 amino acid sequence at least 80% or 90% identical to that set forth in SEQ ID NO: 1 (GFTFSHAWMH), a VH-CDR2 amino acid sequence at least 80%, 90%, or 95% identical to that set forth in SEQ ID NO: 4 (QIKAKSDDYATYYAESVKG), and a VH-CDR3 amino acid sequence at least 80% or 90% identical to that set forth in SEQ ID NO: 8 (TSWEWDLDF). In certain embodiments, described herein is an antibody that specifically binds LIF comprising a VL-CDR1 amino acid sequence at least 80% or 90% identical to that set forth in SEQ ID NO: 9 (RSSQSLLDSDGHTYLN), a VL-CDR2 amino acid sequence at least 80% identical to that set forth in SEQ ID NO: 11 (SVSNLES), and a VL-CDR3 amino acid sequence at least 80% or 90% identical to that set forth in SEQ ID NO: 13 (MQATHAPPYT). In certain embodiments, described herein is an antibody that specifically binds LIF comprising a VH-CDR1 amino acid sequence set forth in SEQ ID NO: 1 (GFTFSHAWMH), a VH-CDR2 amino acid sequence set forth in SEQ ID NO: 4 (QIKAKSDDYATYYAESVKG), a VH-CDR3 amino acid sequence set forth in SEQ ID NO: 8 (TSWEWDLDF), a VL-CDR1 amino acid sequence set forth in SEQ ID NO: 9 (RSSQSLLDSDGHTYLN), a VL-CDR2 amino acid sequence set forth in SEQ ID NO: 11 (SVSNLES), and a VL-CDR3 amino acid sequence set forth in SEQ ID NO: 13 (MQATHAPPYT). Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 1, 4, 8, 9, 11, and 13 by 1, 2, 3, or 4 amino acids. In certain embodiments, the antibody comprises CDRs that differ from the amino acid sequence set forth in any one of SEQ ID NOs: 1, 4, 8, 9, 11, and 13 by 1, 2, 3, or 4 amino acids and does not affect the binding affinity by greater than 10%, 20%, or 30%. In certain embodiments, antibodies that specifically bind LIF comprise one or more human heavy chain framework regions comprising: a VH-FR1 amino acid sequence at least about 80%, 90%, 95%, 97%, 98%, or 99% identical to the amino acid sequence set forth in any one of SEQ ID NOs: 14-17, a VH-FR2 amino acid sequence at least about 80%, 90%, 95%, 97%, 98%, or 99% identical to the amino acid sequence set forth in any one of SEQ ID NOs: 18 or 19, a VH-FR3 amino acid sequence at least 90% identical to the amino acid sequence set forth in any one of SEQ ID NOs: 20-22, or a VH-FR4 region amino acid sequence at least about 80%, 90%, 95%, 97%, 98%, or 99% identical to the amino acid sequence set forth in any one of SEQ ID NOs: 23-25. In certain embodiments, the one or more human heavy chain framework regions comprises a VH-FR1 amino acid sequence at least about 80%, 90%, 95%, 97%, 98%, or 99% identical to the amino acid sequence set forth in SEQ ID NO: 15, a VH-FR2 amino acid sequence at least about 80%, 90%, 95%, 97%, 98%, or 99% identical to the amino acid sequence set forth in SEQ ID NO: 19, a VH-FR3 amino acid sequence at least about 80%, 90%, 95%, 97%, 98%, or 99% identical to the amino acid sequence set forth in SEQ ID NO: 20, and a VH-FR4 amino acid sequence at least about 80%, 90%, 95%, 97%, 98%, or 99% identical to the amino acid sequence set forth in SEQ ID NO: 24. In certain embodiments, the antibody that specifically binds LIF comprises one or more human light chain framework regions comprising: a VL-FR1 amino acid sequence at least about 80%, 90%, 95%, 97%, 98%, or 99% identical to the amino acid sequence set forth in any one of SEQ ID NOs: 26-29, a VL-FR2 amino acid sequence at least about 80%, 90%, 95%, 97%, 98%, or 99% identical to the amino acid sequence set forth in any one of SEQ ID NOs: 30-33, a VL-FR3 amino acid sequence at least about 80%, 90%, 95%, 97%, 98%, or 99% identical to the amino acid sequence set forth in any one of SEQ ID NOs: 34-37, or a VL-FR4 amino acid sequence at least about 80%, 90%, 95%, 97%, 98%, or 99% identical to the amino acid sequence set forth in any one of SEQ ID NOs: 38-40. In certain embodiments, the one or more human light chain framework regions comprise a VL-FR1 amino acid sequence at least about 80%, 90%, 95%, 97%, 98%, or 99% identical to the amino acid sequence set forth in SEQ ID NO: 27, a VL-FR2 amino acid sequence at least about 80%, 90%, 95%, 97%, 98%, or 99% identical to the amino acid sequence set forth in SEQ ID NO: 31, a VL-FR3 amino acid sequence at least about 80%, 90%, 95%, 97%, 98%, or 99% identical to the amino acid sequence set forth in SEQ ID NO: 35, and a VL-FR4 amino acid sequence at least 90% identical to the amino acid sequence set forth in SEQ ID NO: 38. In certain embodiments, the one or more human heavy chain framework regions and the one or more human light chain regions comprise all of a VH-FR1 amino acid sequence at least about 80%, 90%, 95%, 97%, 98%, or 99% identical to the amino acid sequence set forth in SEQ ID NO: 15, a VH-FR2 amino acid sequence at least about 80%, 90%, 95%, 97%, 98%, or 99% identical to the amino acid sequence set forth in SEQ ID NO: 19, a VH-FR3 amino acid sequence at least about 80%, 90%, 95%, 97%, 98%, or 99% identical to the amino acid sequence set forth in SEQ ID NO: 20, a VH-FR4 amino acid sequence at least 90% identical to the amino acid sequence set forth in SEQ ID NO: 24, a VL-FR1 amino acid sequence at least about 80%, 90%, 95%, 97%, 98%, or 99% identical to the amino acid sequence set forth in SEQ ID NO: 27, a VL-FR2 amino acid sequence at least about 80%, 90%, 95%, 97%, 98%, or 99% identical to the amino acid sequence set forth in SEQ ID NO: 31, a VL-FR3 amino acid sequence at least about 80%, 90%, 95%, 97%, 98%, or 99% identical to the amino acid sequence set forth in SEQ ID NO: 35, and a VL-FR4 amino acid sequence at least about 80%, 90%, 95%, 97%, 98%, or 99% identical to the amino acid sequence set forth in SEQ ID NO: 38. In certain embodiments, the antibody specifically binds human LIF. In certain embodiments, the antibody that specifically binds LIF comprises one or more human heavy chain framework regions comprising: a VH-FR1 amino acid sequence identical to the amino acid sequence set forth in any one of SEQ ID NOs: 14-17, a VH-FR2 amino acid sequence identical to the amino acid sequence set forth in any one of SEQ ID NOs: 18 or 19, a VH-FR3 amino acid sequence identical to the amino acid sequence set forth in any one of SEQ ID NOs: 20-22, or a VH-FR4 region amino acid sequence identical to the amino acid sequence set forth in any one of SEQ ID NOs: 23-25. In certain embodiments, the one or more human heavy chain framework regions comprise a VH-FR1 amino acid sequence identical to the amino acid sequence set forth in SEQ ID NO: 15, a VH-FR2 amino acid sequence identical to the amino acid sequence set forth in SEQ ID NO: 19, a VH-FR3 amino acid sequence identical to the amino acid sequence set forth in SEQ ID NO: 20, and a VH-FR4 amino acid sequence identical to the amino acid sequence set forth in SEQ ID NO: 24. In certain embodiments, the antibody that specifically binds LIF comprises one or more human light chain framework regions comprising: a VL-FR1 amino acid sequence identical to the amino acid sequence set forth in any one of SEQ ID NOs: 26-29, a VL-FR2 amino acid sequence identical to the amino acid sequence set forth in any one of SEQ ID NOs: 30-33, a VL-FR3 amino acid sequence identical to the amino acid sequence set forth in any one of SEQ ID NOs: 34-37, or a VL-FR4 amino acid sequence identical to the amino acid sequence set forth in any one of SEQ ID NOs: 38-40. In certain embodiments, the one or more human light chain framework regions comprise a VL-FR1 amino acid sequence identical to the amino acid sequence set forth in SEQ ID NO: 27, a VL-FR2 amino acid sequence identical to the amino acid sequence set forth in SEQ ID NO: 31, a VL-FR3 amino acid sequence identical to the amino acid sequence set forth in SEQ ID NO: 35, and a VL-FR4 amino acid sequence identical to the amino acid sequence set forth in SEQ ID NO: 38. In certain embodiments, the one or more human heavy chain framework regions and the one or more human light chain regions comprise a VH-FR1 amino acid sequence identical to the amino acid sequence set forth in SEQ ID NO: 15, a VH-FR2 amino acid sequence identical to the amino acid sequence set forth in SEQ ID NO: 19, a VH-FR3 amino acid sequence identical to the amino acid sequence set forth in SEQ ID NO: 20, a VH-FR4 amino acid sequence identical to the amino acid sequence set forth in SEQ ID NO: 24, a VL-FR1 amino acid sequence identical to the amino acid sequence set forth in SEQ ID NO: 27, a VL-FR2 amino acid sequence identical to the amino acid sequence set forth in SEQ ID NO: 31, a VL-FR3 amino acid sequence identical to the amino acid sequence set forth in SEQ ID NO: 35, and a VL-FR4 amino acid sequence identical to the amino acid sequence set forth in SEQ ID NO: 38. In certain embodiments, the antibody specifically binds human LIF.

In certain embodiments, described herein is an antibody that specifically binds LIF comprising a humanized heavy chain variable region comprising an amino acid sequence at least about 80%, about 90%, about 95%, about 97%, about 98%, or about 99% identical to the amino acid sequence set forth in any one of SEQ ID NOs: 41, 42, and 44. In certain embodiments, described herein is an antibody that specifically binds LIF comprising a humanized heavy chain variable region comprising an amino acid sequence set forth in any one of SEQ ID NOs: 41, 42, and 44. In certain embodiments, described herein is an antibody that specifically binds LIF comprising a humanized light chain variable region comprising an amino acid sequence at least about 80%, about 90%, about 95%, about 97%, about 98%, or about 99% identical to the amino acid sequence set forth in any one of SEQ ID NOs: 45-48. In certain embodiments, described herein is an antibody that specifically binds LIF comprising a humanized light chain variable region comprising an amino acid sequence set forth in any one of SEQ ID NOs: 45-48. In certain embodiments, the antibody specifically binds human LIF.

In certain embodiments, described herein is an antibody that specifically binds LIF comprising a humanized heavy chain variable region comprising an amino acid sequence at least about 80%, about 90%, about 95%, about 97%, about 98%, or about 99% identical to the amino acid sequence set forth in SEQ ID NO:42; and a humanized light chain variable region comprising an amino acid sequence at least about 80%, about 90%, about 95%, about 97%, about 98%, or about 99% identical to the amino acid sequence set forth in SEQ ID NO: 46. In certain embodiments, described herein is an antibody that specifically binds LIF comprising a humanized heavy chain variable region comprising an amino acid sequence set forth in SEQ ID NO: 42; and a humanized light chain variable region comprising an amino acid sequence set forth in SEQ ID NO: 46.

In certain embodiments, described herein is an antibody that specifically binds LIF comprising a humanized heavy chain variable region comprising an amino acid sequence at least about 80%, about 90%, about 95%, about 97%, about 98%, or about 99% identical to the amino acid sequence set forth in SEQ ID NO: 66; and a humanized light chain variable region comprising an amino acid sequence at least about 80%, about 90%, about 95%, about 97%, about 98%, or about 99% identical to the amino acid sequence set forth in SEQ ID NO: 46. In certain embodiments, described herein is an antibody that specifically binds LIF comprising a humanized heavy chain variable region comprising an amino acid sequence set forth in SEQ ID NO: 66; and a humanized light chain variable region comprising an amino acid sequence set forth in SEQ ID NO: 46.

In certain embodiments, described herein is an antibody that specifically binds LIF comprising a humanized heavy chain comprising an amino acid sequence at least about 80%, about 90%, about 95%, about 97%, about 98%, or about 99% identical to the amino acid sequence set forth in any one of SEQ ID NOs: 57-60; and a humanized light chain comprising an amino acid sequence at least about 80%, about 90%, about 95%, about 97%, about 98%, or about 99% identical to the amino acid sequence set forth in any one of SEQ ID NOs: 61-64. In certain embodiments, described herein is an antibody that specifically binds LIF comprising a humanized heavy chain comprising an amino acid sequence set forth in any one of SEQ ID NOs: 57-60; and a humanized light chain comprising an amino acid sequence set forth in any one of SEQ ID NOs: 61-64.

In certain embodiments, described herein is an antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 58; and a humanized light chain comprising an amino acid sequence at least about 80%, about 90%, about 95%, about 97%, about 98%, or about 99% identical to the amino acid sequence set forth in SEQ ID NO: 62. In certain embodiments, described herein is an antibody that specifically binds LIF comprising a humanized heavy chain comprising an amino acid sequence set forth in SEQ ID NO: 58; and a humanized light chain comprising an amino acid sequence set forth in SEQ ID NO: 62. In certain embodiments, described herein is an antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO:67; and a humanized light chain comprising an amino acid sequence at least about 80%, about 90%, about 95%, about 97%, about 98%, or about 99% identical to the amino acid sequence set forth in SEQ ID NO: 62. In certain embodiments, described herein is an antibody that specifically binds LIF comprising a humanized heavy chain comprising an amino acid sequence set forth in SEQ ID NO: 67; and a humanized light chain comprising an amino acid sequence set forth in SEQ ID NO: 62.

In a certain embodiments, described herein is a recombinant antibody that specifically binds Leukemia Inhibitory Factor (LIF) comprising: a heavy chain complementarity determining region 1 (VH-CDR1) comprising an amino acid sequence set forth in SEQ ID NO: 3; a heavy chain complementarity determining region 2 (VH-CDR2) comprising an amino acid sequence set forth in SEQ ID NO: 4; a heavy chain complementarity determining region 3 (VH-CDR3) comprising an amino acid sequence set forth in SEQ ID NO: 7; a light chain complementarity determining region 1 (VL-CDR1) comprising an amino acid sequence set forth in SEQ ID NO: 9; and a light chain complementarity determining region 2 (VL-CDR2) comprising an amino acid sequence set forth in SEQ ID NO: 11; and a light chain complementarity determining region 3 (VL-CDR3) comprising an amino acid sequence set forth in SEQ ID NO: 13.

In a certain embodiments, described herein is a recombinant antibody that specifically binds Leukemia Inhibitory Factor (LIF) comprising: a heavy chain complementarity determining region 1 (VH-CDR1) comprising an amino acid sequence set forth in SEQ ID NO: 2; a heavy chain complementarity determining region 2 (VH-CDR2) comprising an amino acid sequence set forth in SEQ ID NO: 5; a heavy chain complementarity determining region 3 (VH-CDR3) comprising an amino acid sequence set forth in SEQ ID NO: 6; a light chain complementarity determining region 1 (VL-CDR1) comprising an amino acid sequence set forth in SEQ ID NO: 10; and a light chain complementarity determining region 2 (VL-CDR2) comprising an amino acid sequence set forth in SEQ ID NO: 12; and a light chain complementarity determining region 3 (VL-CDR3) comprising an amino acid sequence set forth in SEQ ID NO: 13. Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 2, 5, 6, 10, 12, and 13 by 1, 2, 3, or 4 amino acids. In certain embodiments, the antibody comprises CDRs that differ from the amino acid sequence set forth in any one of SEQ ID NOs: 2, 5, 6, 10, 12, and 13 by 1, 2, 3, or 4 amino acids and does not affect the binding affinity by greater than 10%, 20%, or 30%.

In a certain embodiments, described herein is a recombinant antibody that specifically binds Leukemia Inhibitory Factor (LIF) comprising: a heavy chain complementarity determining region 1 (VH-CDR1) comprising an amino acid sequence set forth in SEQ ID NO: 3; a heavy chain complementarity determining region 2 (VH-CDR2) comprising an amino acid sequence set forth in SEQ ID NO: 4; a heavy chain complementarity determining region 3 (VH-CDR3) comprising an amino acid sequence set forth in SEQ ID NO: 7; a light chain complementarity determining region 1 (VL-CDR1) comprising an amino acid sequence set forth in SEQ ID NO: 9; and a light chain complementarity determining region 2 (VL-CDR2) comprising an amino acid sequence set forth in SEQ ID NO: 11; and a light chain complementarity determining region 3 (VL-CDR3) comprising an amino acid sequence set forth in SEQ ID NO: 13. Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 3, 4, 7, 9, 11, and 13 by 1, 2, 3, or 4 amino acids. In certain embodiments, the antibody comprises CDRs that differ from the amino acid sequence set forth in any one of SEQ ID NOs: 3, 4, 7, 9, 11, and 13 by 1, 2, 3, or 4 amino acids and does not affect the binding affinity by greater than 10%, 20%, or 30%.

In certain embodiments, described herein is an antibody that specifically binds LIF comprising a humanized heavy chain comprising an amino acid sequence at least about 80%, about 90%, about 95%, about 97%, about 98%, or about 99% identical to the amino acid sequence set forth in any one of SEQ ID NOs: 49-52; and a humanized light chain comprising an amino acid sequence at least about 80%, about 90%, about 95%, about 97%, about 98%, or about 99% identical to the amino acid sequence set forth in any one of SEQ ID NOs: 53-56. In certain embodiments, described herein is an antibody that specifically binds LIF comprising a humanized heavy chain comprising an amino acid sequence set forth in any one of SEQ ID NOs: 49-52; and a humanized light chain comprising an amino acid sequence set forth in any one of SEQ ID NOs: 53-56.

In certain embodiments, described herein is an antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 50; and a humanized light chain comprising an amino acid sequence at least about 80%, about 90%, about 95%, about 97%, about 98%, or about 99% identical to the amino acid sequence set forth in of SEQ ID NO: 54. In certain embodiments, described herein is an antibody that specifically binds LIF comprising a humanized heavy chain comprising an amino acid sequence set forth in SEQ ID NO: 50; and a humanized light chain comprising an amino acid sequence set forth in any one of SEQ ID NO: 54.

### Epitopes Bound by Therapeutically Useful LIF Antibodies

Described herein is a unique epitope of human LIF that when bound inhibits LIF biological activity (e.g., STAT3 phosphorylation) and inhibits tumor growth in vivo. The epitope described herein consists of two discontinuous stretches of amino acids (from residue 13 to residue 32 and from residue 120 to 138 of human LIF), that are present in two distinct topological domains (alpha helixes A and C) of the human LIF protein. This binding is a combination of weak (Van der Waals attraction), medium (hydrogen binding), and strong (salt bridge) interactions. In certain embodiments, a contact residue is a residue on LIF that forms a hydrogen bond with a residue on an anti-LIF antibody. In certain embodiments, a contact residue is a residue on LIF that forms a salt bridge with a residue on an anti-LIF antibody. In certain embodiments, a contact residue is a residue on LIF that results in a Van der Waals attraction with and is within at least 5, 4, or 3 angstroms of a residue on an anti-LIF antibody.

In certain embodiments, the methods and compositions comprising a LIF binding antibody and cisplatin, described herein include an isolated antibody that binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an isolated antibody that binds all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an isolated antibody that binds all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that participate with the antibody in strong or medium interactions. In certain embodiments, the antibody only binds residues that participate with the antibody in strong interactions. In a certain embodiment, the antibody interacts with helix A and C of LIF. In a certain embodiment, the antibody blocks LIF interaction with gp130.

In certain embodiments, described herein is an antibody comprising CDRs with an amino acid sequence set forth in any one of SEQ ID NOs: 1, 4, 6, 9, 11, and 13 that binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an antibody comprising CDRs with an amino acid sequence set forth in any one of SEQ ID NOs: 1, 4, 6, 9, 11, and 13 that binds to all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that participate with the antibody in strong or medium interactions. In certain embodiments, the antibody only binds residues that participate with the antibody in strong interactions.

In certain embodiments, described herein is an antibody comprising CDRs with an amino acid sequence set forth in any one of SEQ ID NOs: 1, 4, 8, 9, 11, and 13 that binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an antibody comprising CDRs with an amino acid sequence set forth in any one of SEQ ID NOs: 1, 4, 8, 9, 11, and 13 that binds to all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that participate with the antibody in strong or medium interactions. In certain embodiments, the antibody only binds residues that participate with the antibody in strong interactions.

In certain embodiments, described herein is an antibody comprising CDRs that differ from the amino acid sequence set forth in any one of SEQ ID NOs: 1, 4, 6, 9, 11, and 13 by 1, 2, 3, 4, or 5 amino acids and binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an antibody comprising CDRs that differ from the amino acid sequence set forth in any one of SEQ ID NOs: 1, 4, 6, 9, 11, and 13 by 1, 2, 3, 4, or 5 amino acids and binds to all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that participate with the antibody in strong or medium interactions. In certain embodiments, the antibody only binds residues that participate with the antibody in strong interactions.

In certain embodiments, described herein is an antibody comprising CDRs that differ from the amino acid sequence set forth in any one of SEQ ID NOs: 1, 4, 8, 9, 11, and 13 by 1, 2, 3, 4, or 5 amino acids and binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an antibody comprising CDRs that differ from the amino acid sequence set forth in any one of SEQ ID NOs: 1, 4, 8, 9, 11, and 13 by 1, 2, 3, 4, or 5 amino acids and bind to all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that that participate with the antibody in strong or medium interactions. In certain embodiments, the antibody only binds residues that that participate with the antibody in strong interactions.

In certain embodiments, described herein is an antibody that specifically binds LIF comprising a humanized heavy chain variable region amino acid sequence at least about 80%, about 90%, about 95%, about 97%, about 98%, or about 99% identical to the amino acid sequence set forth in SEQ ID NO:42; and a humanized light chain variable region amino acid sequence at least about 80%, about 90%, about 95%, about 97%, about 98%, or about 99% identical to the amino acid sequence set forth in SEQ ID NO: 46 and binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an antibody that specifically binds LIF comprising a humanized heavy chain variable region amino acid sequence at least about 80%, about 90%, about 95%, about 97%, about 98%, or about 99% identical to the amino acid sequence set forth in SEQ ID NO:42; and a humanized light chain variable region amino acid sequence at least about 80%, about 90%, about 95%, about 97%, about 98%, or about 99% identical to the amino acid sequence set forth in SEQ ID NO: 46 and binds all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that that participate with the antibody in strong or medium interactions. In certain embodiments, the antibody only binds residues that that participate with the antibody in strong interactions.

In certain embodiments, described herein is an antibody that specifically binds LIF comprising a humanized heavy chain variable region amino acid sequence at least about 80%, about 90%, about 95%, about 97%, about 98%, or about 99% identical to the amino acid sequence set forth in SEQ ID NO: 66; and a humanized light chain variable region amino acid sequence at least about 80%, about 90%, about 95%, about 97%, about 98%, or about 99% identical to the amino acid sequence set forth in SEQ ID NO: 46 and binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, described herein is an antibody that specifically binds LIF comprising a humanized heavy chain variable region amino acid sequence at least about 80%, about 90%, about 95%, about 97%, about 98%, or about 99% identical to the amino acid sequence set forth in SEQ ID NO: 66; and a humanized light chain variable region amino acid sequence at least about 80%, about 90%, about 95%, about 97%, about 98%, or about 99% identical to the amino acid sequence set forth in SEQ ID NO: 46 and binds all of the following residues: A13, I14, R15, H16, P17, C18, H19, N20, Q25, Q29, Q32, D120, R123, S127, N128, L130, C131, C134, S135, or H138 of SEQ ID NO: 68. In certain embodiments, the antibody only binds residues that that participate with the antibody in strong or medium interactions. In certain embodiments, the antibody only binds residues that that participate with the antibody in strong interactions.

In certain embodiments, the antibodies disclosed herein inhibit LIF signaling in cells. In certain embodiments, the IC₅₀ for biological inhibition of the antibody under serum starved conditions in U-251 cells is less than or equal to about 100, 75, 50, 40, 30, 20, 10, 5, or 1 nanomolar. In certain embodiments, the IC₅₀ for biological inhibition of the antibody under serum starved conditions in U-251 cells is less than or equal to about 900, 800, 700, 600, 500, 400, 300, 200, or 100 nanomolar.

In certain embodiments, the antibodies disclosed herein, are useful for treating tumors and cancers that express LIF. In certain embodiments, an individual treated with the antibodies of this disclosure has been selected for treatment as having a LIF positive tumor/cancer. In certain embodiments, the tumor is LIF positive or produces elevated levels of LIF. In certain embodiments, LIF positivity is determined in comparison to a reference value or a set of pathological criteria. In certain embodiments, a LIF positive tumor expresses greater than 2-fold, 3-fold, 5-fold, 10-fold, 100-fold or more LIF than a non-transformed cell from which the tumor is derived. In certain embodiments, the tumor has acquired ectopic expression of LIF. A LIF positive tumor can be determined histologically using, for example, immunohistochemistry with an anti-LIF antibody; by commonly used molecular biology methods such as, for example, mRNA quantitation by real-time PCR or RNA-seq; or protein quantitation, for example, by western blot, flow cytometry, ELISA, or a homogenous protein quantitation assays (e.g., AlphaLISA®). In certain embodiments, the antibodies can be used to treat patients diagnosed with cancer. In certain embodiments, the cancer comprises one or more cancer stem cells or is one or more cancer stem cells.

In certain embodiments, the antibodies disclosed herein, are useful for treating tumors in cancers that express the LIF receptor (CD118). A LIF receptor positive tumor can be determined by histopathology or flow cytometry, and, in certain embodiments, comprises a cell that binds a LIF receptor antibody greater than 2×, 3×, 3×, 4×, 5×, 10× or more than an isotype control. In certain embodiments, the tumor has acquired ectopic expression of the LIF receptor. In a certain embodiment, the cancer is a cancer stem cell. In a certain embodiment, a LIF positive tumor or cancer can be determined by immunohistochemistry using anti-LIF an anti-LIF antibody. In a certain embodiment, a LIF positive tumor is determined by IHC analysis with a LIF Level in the top 10%, 20%, 30%, 40%, or top 50% of tumors.

The antibodies described herein influence numerous outcomes. In a certain embodiment, the antibodies described herein can reduce the presence of M2 macrophages in tumors by at least 10%, 20%, 30%, 40%, 50%, 60%, 70%, 80%, 90% or more in a tumor model compared to a control antibody (e.g., isotype control). M2 macrophages can be identified by staining for CCL22 and CD206 in IHC sections or by flow cytometry of tumor infiltrating immune or myeloid cells. In a certain embodiment, the antibodies described herein can reduce the binding of LIF to gp130 tumors by at least 10%, 20%, 30%, 40%, 50%, 60%, 70%, 80%, 90% or more when compared to a control antibody (e.g., isotype control). In a certain embodiment, the antibodies described herein can reduce LIF signaling by at least 10%, 20%, 30%, 40%, 50%, 60%, 70%, 80%, 90% or more in a LIF responsive cell line compared to a control antibody (e.g., isotype control). LIF signaling can be measured by, for example, western blot for phosphorylated STAT3 (a downstream target of LIF signaling). The antibodies here are also highly specific for LIF compared to other IL-6 family member cytokines. In certain embodiments, the antibodies bind human LIF with an affinity about 10×, about 50×, or about 100× greater than that of any other IL-6 family member cytokine. In certain embodiments, the LIF antibodies do not bind to other IL-6 family member cytokines that are produced in a mammalian system. In certain embodiments, the antibodies do not bind to Oncostatin M that has been produced in a mammalian system.

In certain embodiments, the LIF-binding polypeptides and antibodies can be administered by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered on a suitable dosage schedule, for example, weekly, twice weekly, monthly, twice monthly, etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. A LIF-binding polypeptide or antibody can be administered i.v. over a time period of at least about 60 minutes; however, this period can vary somewhat based upon conditions relevant to each individual administration.

### Platinum-Based Antineoplastic Agent

Platinum-based antineoplastic agents, also known as platins, are coordination complexes of platinum that are used to treat cancer. The LIF-binding polypeptides and antibodies described herein can be combined with a platinum-based antineoplastic agent and deployed in a method to treat a tumor, cancer or other neoplasm. In certain embodiments, the LIF-binding polypeptides and antibodies described herein can be combined with a platinum-based antineoplastic agent in a pharmaceutical composition useful for treating a cancer, tumor, or other neoplasm. The h5D8 antibody described herein can be combined with a platinum-based antineoplastic agent and deployed in a method to treat a tumor, cancer or other neoplasm. In certain embodiments, the h5D8 antibody described herein can be combined with a platinum-based antineoplastic agent in a pharmaceutical composition useful for treating a cancer, tumor, or other neoplasm.

The platinum-based antineoplastic agents utilized in the compositions and methods herein may cause crosslinking of DNA or binding with DNA. The crosslinking may inhibit DNA repair or DNA synthesis in cancer cells. The crosslinking may impede with cellular process and lead to apoptosis. In some embodiments, the platinum-based antineoplastic agents comprise: cisplatin (Platinol®, cis-diamminedichloridoplatinum(II) (CDDP), cis-diammineplatinum(II) dichloride, [SP-4-2]-diaminedichloroplatinum); carboplatin (Paraplatin®, cis-diammine(1,1-cyclobutanedicarboxylato)platinum); oxaliplatin (Eloxatin®, [SP-4-2]-(1R-trans)]-(1,2-cyclohexanediamine-N,N′)[ethanedioata(2−)-O,O′]platinum); nedaplatin (Aqupla®, cis-diammine(glycolato-O,O′)platinum, [SP-4-3]-diammine(hydroxyacetato(2−)-O¹,O²)platinum); triplatin tetranitrate (BBR3464, hexaamminedichloro-bis[μ-(1,6-hexanediamine-κN:κN′)]tri-platinum(4⁺) tetranitrate); phenathriplatin ([SP-4-3]-diamminechloro(phenanthridine)-platinum(1⁺) nitrate, picoplatin ([SP-4-3]-amminedichloro(2-methylpyridine)platinum), satraplatin (JM216, [OC-6-43]-bis(acetato-O) amminedichloro(cyclohexanamine)platinum); lobaplatin ([SP-4-3 -(S),(trans)]-(1,2-cyclobutanedimethanamine-N,N′)[2-hydroxypropanoato(2−)-O¹,O²]-platinum); heptaplatin (Eptaplatin, [SP-4-2-[4R-(2α,4α,5β)]]-[2-(1-methylethyl)-1,3-dioxolane-4,5-dimethanamine-N,N′][propanedioato(2−)-O,O′]-platinum); pharmaceutically acceptable salts thereof, or combinations thereof. In some embodiments, the platinum-based antineoplastic agent is cisplatin.

In certain embodiments, the platinum-based antineoplastic agents can be administered by any route suitable for the administration of a small molecule-containing pharmaceutical composition, such as, for example, subcutaneous, intraperitoneal, intravenous, intramuscular, intratumoral, intracerebral, or oral. In certain embodiments, platinum-based antineoplastic agents are administered intravenously. In certain embodiments, the platinum-based antineoplastic agents are administered on a suitable dosage schedule, for example, daily, once every two days, once every three days, once every four days, once every five days, once every six days, weekly, twice weekly, monthly, twice monthly, once every two weeks, once every three weeks, or once every four weeks. The platinum-based antineoplastic agents can be administered in any therapeutically effective amount. In certain embodiments, the therapeutically acceptable amount is between about 1 mg/m² to about 1000 mg/m² body surface area. In certain embodiments, the therapeutically acceptable amount is between about 50 mg/m² to about 70 mg/m². In certain embodiments, the therapeutically acceptable amount is between about 50 mg/m² to about 100 mg/m², about 50 mg/m² to about 150 mg/m², or about 50 mg/m² to about 200 mg/m² body surface area. In certain embodiments, the therapeutically acceptable amount is between about 10 mg/m² to about 50 mg/m², about 10 mg/m² to about 70 mg/m², or 10 mg/m² to about 100 mg/m² body surface area. In certain embodiments, the therapeutically acceptable amount is between about 1 mg/m² to about 50 mg/m², about 1 mg/m² to about 70 mg/m², or about 1 mg/m² to about 100 mg/m² body surface area. In certain embodiments, the therapeutically acceptable amount is between about 100 mg/m² to about 200 mg/m², about 200 mg/m² to about 500 mg/m², or about 300 mg/m² to about 600 mg/m² body surface area. In certain embodiments, the therapeutically acceptable amount is about 1 mg/m², 2 mg/m², 3 mg/m², 4 mg/m², 5 mg/m², 6 mg/m², 7 mg/m², 8 mg/m², 9 mg/m², 10 mg/m², 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 300 mg/m², 400 mg/m², 500 mg/m², 600 mg/m², 700 mg/m², 800 mg/m², 900 mg/m², or 1000 mg/m² body surface area.

In certain embodiments, cisplatin can be administered in any therapeutically effective amount. In certain embodiments, the therapeutically acceptable amount of cisplatin is between about 50 mg/m² to about 70 mg/m² body surface area. In certain embodiments, the therapeutically acceptable amount of cisplatin is between about 50 mg/m² to about 100 mg/m² body surface area. In certain embodiments, the therapeutically acceptable amount of cisplatin is between about 10 mg/m² to about 70 mg/m² body surface area. In certain embodiments, the therapeutically acceptable amount of cisplatin is between about 10 mg/m² to about 100 mg/m² body surface area. In certain embodiments, the therapeutically acceptable amount of cisplatin is between about 1 mg/m² to about 50 mg/m² body surface area. In certain embodiments, the therapeutically acceptable amount of cisplatin is between about 1 mg/m² to about 70 mg/m² body surface area. In certain embodiments, the therapeutically acceptable amount of cisplatin is between about 1 mg/m² to about 100 mg/m² body surface area. In certain embodiments, the therapeutically acceptable amount of cisplatin is about 1 mg/m², 2 mg/m², 3 mg/m², 4 mg/m², 5 mg/m², 6 mg/m², 7 mg/m², 8 mg/m², 9 mg/m², 10 mg/m², 20 mg/m², 30 mg/m², 40 mg/m², 50 mg/m², 60 mg/m², 70 mg/m², 80 mg/m², 90 mg/m², or 100 mg/m² body surface area.

In certain embodiments, administration to an individual of the platinum-based antineoplastic agents can be at a flat dosage level of between about 10 milligrams and about 1500 milligrams. In certain embodiments, administration to an individual of the platinum-based antineoplastic agents can be at flat dosage level of between about 50 milligrams and about 1000 milligrams, about 50 milligrams and about 800 milligrams, between about 50 milligrams and about 600 milligrams, between about 50 milligrams and about 500 milligrams, between about 100 milligrams and about 500 milligrams. In certain embodiments, administration to an individual of the platinum-based antineoplastic agents can be at a flat dosage level of about 10, 20, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, or 1500 milligrams. In certain embodiments, administration to an individual of the platinum-based antineoplastic agents can be at level suitable for monotherapy.

In certain embodiments, administration to an individual of cisplatin can be at a flat dosage level of between about 10 milligrams and about 500 milligrams. In certain embodiments, administration to an individual of cisplatin can be at a flat dosage level of between about 50 milligrams and about 500 milligrams, about 50 milligrams and about 400 milligrams, about 100 milligrams and about 400 milligrams, about 100 milligrams and about 300 milligrams, or about 100 milligrams and about 200 milligrams. In certain embodiments, administration to an individual of cisplatin can be at a flat dosage level of about 10, 20, 30, 40, 50, 60, 70, 80, 90, 100, 150, 200, 250, 300, 350, 400, 450, or 500 milligrams. In certain embodiments, administration to an individual of cisplatin can be at level suitable for monotherapy.

### H5D8 Dosages

In certain embodiments, the antibodies can be administered by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered on a suitable dosage schedule, for example, weekly, twice weekly, monthly, twice monthly, etc. In certain embodiments, the antibodies are administered once every three weeks. The antibodies can be administered in any therapeutically effective amount. In certain embodiments, the therapeutically acceptable amount is between about 0.01 mg/kg and about 50 mg/kg. In certain embodiments, the therapeutically acceptable amount is between about 0.05 mg/kg and about 50 mg/kg. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. The h5D8 antibody can be administered at a flat dose regardless of the weight or mass of the individual to whom the h5D8 antibody is administered. The h5D8 antibody can be administered at a flat dose regardless of the weight or mass of the individual to whom the h5D8 antibody is administered, provided that the individual has a mass of at least about 37.5 kilograms. A flat dose of h5D8 can be administered from about 1 milligram to about 2000 milligrams. A flat dose of h5D8 can be administered from about 10 milligrams to about 2000 milligrams. A flat dose of h5D8 can be administered from about 25 milligrams to about 2000 milligrams. A flat dose of h5D8 can be administered from about 50 milligrams to about 2000 milligrams. A flat dose of h5D8 can be administered from about 75 milligrams to about 2000 milligrams. A flat dose of h5D8 can be administered from about 225 milligrams to about 2000 milligrams, from about 750 milligrams to about 2000 milligrams, from about 1125 milligrams to about 2000 milligrams, or from about 1500 milligrams to about 2000 milligrams. A flat dose of h5D8 can be administered at about 1 milligram. A flat dose of h5D8 can be administered at about 10 milligrams. A flat dose of h5D8 can be administered at about 25 milligrams. A flat dose of h5D8 can be administered at about 50 milligrams. A flat dose of h5D8 can be administered at about 75 milligrams. A flat dose of h5D8 can be administered at about 225 milligrams. A flat dose of h5D8 can be administered at about 750 milligrams. A flat dose of h5D8 can be administered at about 1125 milligrams. A flat dose of h5D8 can be administered at about 1500 milligrams. A flat dose of h5D8 can be administered at about 2000 milligrams.

Other dosages of h5D8 are contemplated. A flat dose of h5D8 can be administered at about 1, 10, 25, 50, 100, 150, 175, 200, 250, 300, 325, 350, 375, 400, 425, 450, 475, 500, 525, 550, 575, 600, 625, 650, 675, 700, 725, 775, 800, 825, 850, 875, 900, 925, 950, 975, 1000, 1025, 1050, 1075, 1100, 1150, 1175, 1200, 1225, 1250, 1275, 1300, 1325, 1350, 1375, 1400, 1425, 1450, 1475, 1525, 1550, 1575, 1600, 1625, 1650, 1675, 1700, 1725, 1750, 1775, 1800, 1825, 1850, 1875, 1900, 1925, 1950, 1975, 2025, 2050, 2075, or 2100 milligrams. Any of these doses can be administered once a week, once every two weeks, once every three weeks, or once every four weeks.

A flat dose of h5D8 can be administered from about 1 milligram to about 2000 milligrams once a week. A flat dose of h5D8 can be administered from about 10 milligrams to about 2000 milligrams once a week. A flat dose of h5D8 can be administered from about 25 milligrams to about 2000 milligrams once a week. A flat dose of h5D8 can be administered from about 50 milligrams to about 2000 milligrams once a week. A flat dose of h5D8 can be administered from about 75 milligrams to about 2000 milligrams once a week. A flat dose of h5D8 can be administered from about 75 milligrams to about 1500 milligrams once a week. A flat dose of h5D8 can be administered from about 225 milligrams to about 1500 milligrams, from about 750 milligrams to about 1500 milligrams, from about 1125 milligrams to about 1500 milligrams once a week. A flat dose of h5D8 can be administered at about 1 milligram once a week. A flat dose of h5D8 can be administered at about 10 milligrams once a week. A flat dose of h5D8 can be administered at about 25 milligrams once a week. A flat dose of h5D8 can be administered at about 50 milligrams once a week. A flat dose of h5D8 can be administered at about 75 milligrams once a week. A flat dose of h5D8 can be administered at about 225 milligrams once a week. A flat dose of h5D8 can be administered at about 750 milligrams once a week. A flat dose of h5D8 can be administered at about 1125 milligrams once a week. A flat dose of h5D8 can be administered at about 1500 milligrams once a week. A flat dose of h5D8 can be administered at about 2000 milligrams once a week.

A flat dose of h5D8 can be administered from about 1 milligram to about 2000 milligrams once every two weeks. A flat dose of h5D8 can be administered from about 10 milligrams to about 2000 milligrams once every two weeks. A flat dose of h5D8 can be administered from about 25 milligrams to about 2000 milligrams once every two weeks. A flat dose of h5D8 can be administered from about 50 milligrams to about 2000 milligrams once every two weeks. A flat dose of h5D8 can be administered from about 75 milligrams to about 2000 milligrams once every two weeks. A flat dose of h5D8 can be administered from about 75 milligrams to about 1500 milligrams once every two weeks. A flat dose of h5D8 can be administered from about 225 milligrams to about 1500 milligrams, from about 750 milligrams to about 1500 milligrams, from about 1125 milligrams to about 1500 milligrams once every two weeks. A flat dose of h5D8 can be administered at about 1 milligram once every two weeks. A flat dose of h5D8 can be administered at about 10 milligrams once every two weeks. A flat dose of h5D8 can be administered at about 25 milligrams once every two weeks. A flat dose of h5D8 can be administered at about 50 milligrams once every two weeks. A flat dose of h5D8 can be administered at about 75 milligrams once every two weeks. A flat dose of h5D8 can be administered at about 225 milligrams once every two weeks. A flat dose of h5D8 can be administered at about 750 milligrams once every two weeks. A flat dose of h5D8 can be administered at about 1125 milligrams once every two weeks. A flat dose of h5D8 can be administered at about 1500 milligrams once every two weeks. A flat dose of h5D8 can be administered at about 2000 milligrams once every two weeks.

A flat dose of h5D8 can be administered from about 1 milligram to about 2000 milligrams once every three weeks. A flat dose of h5D8 can be administered from about 10 milligrams to about 2000 milligrams once every three weeks. A flat dose of h5D8 can be administered from about 25 milligrams to about 2000 milligrams once every three weeks. A flat dose of h5D8 can be administered from about 50 milligrams to about 2000 milligrams once every three weeks. A flat dose of h5D8 can be administered from about 75 milligrams to about 2000 milligrams once every three weeks. A flat dose of h5D8 can be administered from about 75 milligrams to about 1500 milligrams once every three weeks. A flat dose of h5D8 can be administered from about 225 milligrams to about 1500 milligrams, from about 750 milligrams to about 1500 milligrams, from about 1125 milligrams to about 1500 milligrams once every three weeks. A flat dose of h5D8 can be administered at about 1 milligram once every three weeks. A flat dose of h5D8 can be administered at about 10 milligrams once every three weeks. A flat dose of h5D8 can be administered at about 25 milligrams once every three weeks. A flat dose of h5D8 can be administered at about 50 milligrams once every three weeks. A flat dose of h5D8 can be administered at about 75 milligrams once every three weeks. A flat dose of h5D8 can be administered at about 225 milligrams once every three weeks. A flat dose of h5D8 can be administered at about 750 milligrams once every three weeks. A flat dose of h5D8 can be administered at about 1125 milligrams once every three weeks. A flat dose of h5D8 can be administered at about 1500 milligrams once every three weeks. A flat dose of h5D8 can be administered at about 2000 milligrams once every three weeks.

A flat dose of h5D8 can be administered from about 1 milligram to about 2000 milligrams once every four weeks. A flat dose of h5D8 can be administered from about 10 milligrams to about 2000 milligrams once every four weeks. A flat dose of h5D8 can be administered from about 25 milligrams to about 2000 milligrams once every four weeks. A flat dose of h5D8 can be administered from about 50 milligrams to about 2000 milligrams once every four weeks. A flat dose of h5D8 can be administered from about 75 milligrams to about 2000 milligrams once every four weeks. A flat dose of h5D8 can be administered from about 75 milligrams to about 1500 milligrams once every four weeks. A flat dose of h5D8 can be administered from about 225 milligrams to about 1500 milligrams, from about 750 milligrams to about 1500 milligrams, from about 1125 milligrams to about 1500 milligrams once every four weeks. A flat dose of h5D8 can be administered at about 1 milligram once every four weeks. A flat dose of h5D8 can be administered at about 10 milligrams once every four weeks. A flat dose of h5D8 can be administered at about 25 milligrams once every four weeks. A flat dose of h5D8 can be administered at about 50 milligrams once every four weeks. A flat dose of h5D8 can be administered at about 75 milligrams once every four weeks. A flat dose of h5D8 can be administered at about 225 milligrams once every four weeks. A flat dose of h5D8 can be administered at about 750 milligrams once every four weeks. A flat dose of h5D8 can be administered at about 1125 milligrams once every four weeks. A flat dose of h5D8 can be administered at about 1500 milligrams once every four weeks. A flat dose of h5D8 can be administered at about 2000 milligrams once every four weeks.

The h5D8 antibody can be administered at a dose based on the bodyweight or mass of the individual to whom the h5D8 antibody is administered. A body weight adjusted dose of h5D8 can be administered from about 0.01 mg/kg to about 25 mg/kg. A body weight adjusted dose of h5D8 can be administered from about 0.05 mg/kg to about 25 mg/kg. A body weight adjusted dose of h5D8 can be administered from about 0.1 mg/kg to about 25 mg/kg. A body weight adjusted dose of h5D8 can be administered from about 0.5 mg/kg to about 25 mg/kg. A body weight adjusted dose of h5D8 can be administered from about 1 mg/kg to about 25 mg/kg. A body weight adjusted dose of h5D8 can be administered from about 3 mg/kg to about 25 mg/kg, from about 10 mg/kg to about 25 mg/kg, from about 15 mg/kg to about 25 mg/kg, or from about 20 mg/kg to about 25 mg/kg. A body weight adjusted dose of h5D8 can be administered at about 1 mg/kg. A body weight adjusted dose of h5D8 can be administered at about 3 mg/kg. A body weight adjusted dose of h5D8 can be administered at about 10 mg/kg. A body weight adjusted dose of h5D8 can be administered at about 15 mg/kg. A body weight adjusted dose of h5D8 can be administered at about 20 mg/kg. A body weight adjusted dose of h5D8 can be administered at about 25 mg/kg.

A body weight adjusted dose of h5D8 can be administered from about 0.01 mg/kg to about 25 mg/kg once every three weeks. A body weight adjusted dose of h5D8 can be administered from about 0.05 mg/kg to about 25 mg/kg once every three weeks. A body weight adjusted dose of h5D8 can be administered from about 0.1 mg/kg to about 25 mg/kg once every three weeks. A body weight adjusted dose of h5D8 can be administered from about 0.5 mg/kg to about 25 mg/kg once every three weeks. A body weight adjusted dose of h5D8 can be administered from about 1 mg/kg to about 25 mg/kg once every three weeks. A body weight adjusted dose of h5D8 can be administered from about 3 mg/kg to about 25 mg/kg, from about 10 mg/kg to about 20 mg/kg, from about 15 mg/kg to about 25 mg/kg, or from about 20 mg/kg to about 25 mg/kg once every one, two, three, or four weeks.

Other bodyweight adjusted doses of h5D8 are contemplated. A body weight adjusted dose of h5D8 can be administered at about 0.01 mg/kg, 0.05 mg/kg, 0.1 mg/kg, 0.5 mg/kg, 1 mg/kg, 2 mg/kg, 4 mg/kg, 5 mg/kg, 6 mg/kg, 7 mg/kg, 8 mg/kg, 9 mg/kg, 11 mg/kg, 12 mg/kg, 13 mg/kg, 14 mg/kg, 16 mg/kg, 17 mg/kg, 18 mg/kg, 19 mg/kg, 21 mg/kg, 22 mg/kg, 23 mg/kg, 24 mg/kg, 26 mg/kg, 27 mg/kg, 28 mg/kg, 29 mg/kg, or 30 mg/kg. Any of these doses can be administered once a week, once every two weeks, once every three weeks, or once every four weeks.

A body weight adjusted dose of h5D8 can be administered at about 0.01 mg/kg once a week. A body weight adjusted dose of h5D8 can be administered at about 0.05 mg/kg once a week. A body weight adjusted dose of h5D8 can be administered at about 0.1 mg/kg once a week. A body weight adjusted dose of h5D8 can be administered at about 0.5 mg/kg once a week. A body weight adjusted dose of h5D8 can be administered at about 1 mg/kg once a week. A body weight adjusted dose of h5D8 can be administered at about 3 mg/kg once a week. A body weight adjusted dose of h5D8 can be administered at about 10 mg/kg once a week. A body weight adjusted dose of h5D8 can be administered at about 15 mg/kg once a week. A body weight adjusted dose of h5D8 can be administered at about 20 mg/kg once a week. A body weight adjusted dose of h5D8 can be administered at about 25 mg/kg once a week.

A body weight adjusted dose of h5D8 can be administered at about 0.01 mg/kg once every two weeks. A body weight adjusted dose of h5D8 can be administered at about 0.05 mg/kg once every two weeks. A body weight adjusted dose of h5D8 can be administered at about 0.1 mg/kg once every two weeks. A body weight adjusted dose of h5D8 can be administered at about 0.5 mg/kg once every two weeks. A body weight adjusted dose of h5D8 can be administered at about 1 mg/kg once every two weeks. A body weight adjusted dose of h5D8 can be administered at about 3 mg/kg once every two weeks. A body weight adjusted dose of h5D8 can be administered at about 10 mg/kg once every two weeks. A body weight adjusted dose of h5D8 can be administered at about 15 mg/kg once every two weeks. A body weight adjusted dose of h5D8 can be administered at about 20 mg/kg once every two weeks. A body weight adjusted dose of h5D8 can be administered at about 25 mg/kg once every two weeks.

A body weight adjusted dose of h5D8 can be administered at about 0.01 mg/kg once every three weeks. A body weight adjusted dose of h5D8 can be administered at about 0.05 mg/kg once every three weeks. A body weight adjusted dose of h5D8 can be administered at about 0.1 mg/kg once every three weeks. A body weight adjusted dose of h5D8 can be administered at about 0.5 mg/kg once every three weeks. A body weight adjusted dose of h5D8 can be administered at about 1 mg/kg once every three weeks. A body weight adjusted dose of h5D8 can be administered at about 3 mg/kg once every three weeks. A body weight adjusted dose of h5D8 can be administered at about 10 mg/kg once every three weeks. A body weight adjusted dose of h5D8 can be administered at about 15 mg/kg once every three weeks. A body weight adjusted dose of h5D8 can be administered at about 20 mg/kg once every three weeks. A body weight adjusted dose of h5D8 can be administered at about 25 mg/kg once every three weeks.

A body weight adjusted dose of h5D8 can be administered at about 0.01 mg/kg once every four weeks. A body weight adjusted dose of h5D8 can be administered at about 0.05 mg/kg once every four weeks. A body weight adjusted dose of h5D8 can be administered at about 0.1 mg/kg once every four weeks. A body weight adjusted dose of h5D8 can be administered at about 0.5 mg/kg once every four weeks. A body weight adjusted dose of h5D8 can be administered at about 1 mg/kg once every four weeks. A body weight adjusted dose of h5D8 can be administered at about 3 mg/kg once every four weeks. A body weight adjusted dose of h5D8 can be administered at about 10 mg/kg once every four weeks. A body weight adjusted dose of h5D8 can be administered at about 15 mg/kg once every four weeks. A body weight adjusted dose of h5D8 can be administered at about 20 mg/kg once every four weeks. A body weight adjusted dose of h5D8 can be administered at about 25 mg/kg once every four weeks.

Any of the doses detailed herein can be administered i.v. over a time period of at least about 60 minutes; however, this period can vary somewhat based upon conditions relevant to each individual administration.

### Dosage Schedules of Combination Therapies

A combination treatment comprising a LIF-binding polypeptide and a platinum-based antineoplastic agent can be administered in a variety of ways. The LIF-binding polypeptide and the platinum-based antineoplastic agent can be administered at the same time on the same schedule, or at different times and on different schedules. When administered at the same time the administration can be by way of separate formulations or a single formulation comprising both the LIF-binding polypeptide and the platinum-based antineoplastic agent. Modes of administration can be mixed, for example a LIF-binding polypeptide can be administered intravenously while a platinum-based antineoplastic agent can be administered orally or by parenteral injection. In certain embodiments, a LIF-binding polypeptide is administered intravenously, parenterally, subcutaneously, intratumorally, or orally. In certain embodiments, a platinum-based antineoplastic agent is administered intravenously, parenterally, subcutaneously, intratumorally, or orally.

When a combination treatment is administered to an individual on the same schedule the LIF-binding polypeptide and the platinum-based antineoplastic agent can be administered once every week, once every two weeks, or once every four weeks. The LIF-binding polypeptide and the platinum-based antineoplastic agent can be administered separately or as a single formulation.

When a combination treatment is administered to an individual on a different schedule the LIF-binding polypeptide and the platinum-based antineoplastic agent can be alternated. In certain embodiments, a platinum-based antineoplastic agent can be administered to an individual one or more times before administration of a LIF-biding polypeptide. A LIF-binding polypeptide can be administered within 1 day, 2 days, 3 days, 4 days, 5 days, or 6 days of administration of a platinum-based antineoplastic agent. A LIF-binding polypeptide can be administered within 1 week, 2 weeks, 3 weeks, or 4 weeks of administration of a platinum-based antineoplastic agent. The h5D8 antibody can be administered within 1 day, 2 days, 3 days, 4 days, 5 days, or 6 days of administration of a platinum-based antineoplastic agent. The h5D8 antibody can be administered within 1 week, 2 weeks, 3 weeks, or 4 weeks of administration of a platinum-based antineoplastic agent.

A LIF-biding polypeptide can be administered to an individual one or more times before administration of a platinum-based antineoplastic agent. In certain embodiments, a platinum-based antineoplastic agent can be administered within 1 day, 2 days, 3 days, 4 days, 5 days, or 6 days of administration of a LIF-binding polypeptide. In certain embodiments, a platinum-based antineoplastic agent can be administered within 1 week, 2 weeks, 3 weeks, or 4 weeks of administration of a LIF-binding polypeptide. In certain embodiments, a platinum-based antineoplastic agent can be administered within 1 day, 2 days, 3 days, 4 days, 5 days, or 6 days of administration of the h5D8 antibody. In certain embodiments, a platinum-based antineoplastic agent can be administered within 1 week, 2 weeks, 3 weeks, or 4 weeks of administration of the h5D8 antibody.

In certain embodiments, a LIF binding polypeptide can be administered to an individual once every week and a platinum-based antineoplastic agent can be administered to an individual every week, every two weeks, every three weeks or every four weeks. In certain embodiments, a LIF binding polypeptide can be administered to an individual once every two weeks and a platinum-based antineoplastic agent can be administered to an individual every week, every two weeks, every three weeks or every four weeks. In certain embodiments, a LIF binding polypeptide can be administered to an individual once every three weeks and a platinum-based antineoplastic agent can be administered to an individual every week, every two weeks, every three weeks or every four weeks. In certain embodiments, a LIF binding polypeptide can be administered to an individual once every four weeks and a platinum-based antineoplastic agent can be administered to an individual every week, every two weeks, every three weeks or every four weeks. In certain embodiments, a platinum-based antineoplastic agent can be administered to an individual once every week and a LIF-binding polypeptide can be administered to an individual every week, every two weeks, every three weeks or every four weeks. In certain embodiments, a platinum-based antineoplastic agent can be administered to an individual once every two weeks and a LIF-binding polypeptide can be administered to an individual every week, every two weeks, every three weeks or every four weeks. In certain embodiments, a platinum-based antineoplastic agent can be administered to an individual once every three weeks and a LIF-binding polypeptide can be administered to an individual every week, every two weeks, every three weeks or every four weeks. In certain embodiments, a platinum-based antineoplastic agent can be administered to an individual once every four weeks and a LIF-binding polypeptide can be administered to an individual every week, every two weeks, every three weeks or every four weeks. In certain embodiments, h5D8 can be administered to an individual one or more times before administration of a platinum-based antineoplastic agent. In certain embodiments, h5D8 can be administered to an individual once every week and a platinum-based antineoplastic agent can be administered to an individual every week, every two weeks, every three weeks or every four weeks. In certain embodiments, h5D8 can be administered to an individual once every two weeks and a platinum-based antineoplastic agent can be administered to an individual every week, every two weeks, every three weeks or every four weeks. In certain embodiments, h5D8 can be administered to an individual once every three weeks and a platinum-based antineoplastic agent can be administered to an individual every week, every two weeks, every three weeks or every four weeks. In certain embodiments, h5D8 can be administered to an individual once every four weeks and a platinum-based antineoplastic agent can be administered to an individual every week, every two weeks, every three weeks or every four weeks.

A combination treatment according to the current disclosure may comprise combinations wherein one or both of the activate ingredients (e.g., a LIF-binding polypeptide and a platinum-based antineoplastic agent) is not effective by itself as a monotherapy, but is effective when administered as a part of a combination treatment. In certain embodiments, a platinum-based antineoplastic agent is administered at a level not effective for monotherapy, but effective in combination with a LIF-binding polypeptide. In certain embodiments, a platinum-based antineoplastic agent is administered at a level not effective for monotherapy, but effective in combination with the h5D8 antibody. In certain embodiments, a LIF-binding polypeptide is administered at a level not effective for monotherapy, but effective in combination with a platinum-based antineoplastic agent. In certain embodiments, h5D8 is administered at a level not effective for monotherapy, but effective in combination with a platinum-based antineoplastic agent. In certain embodiments, both a LIF-binding polypeptide, and a platinum-based antineoplastic agent is administered at a level not effective for monotherapy, but is effective in combination. In certain embodiments, both h5D8, and a platinum-based antineoplastic agent is administered at a level not effective for monotherapy, but is effective in combination.

Therapeutic indications

In certain embodiments, disclosed herein, are methods and compositions useful for the treatment of a cancer or tumor. In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VlPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer is a relapsed/refractory glioblastoma, pancreatic cancer, ovarian cancer, colon cancer, prostate cancer, or lung cancer. In certain embodiments, the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer comprises an advanced solid tumor. In certain embodiments, the individual is refractory to previous treatment with a LIF binding antibody as a monotherapy. In certain embodiments, the individual is refractory to previous treatment with a platinum-based antineoplastic agent as a monotherapy.

### Pharmaceutically Acceptable Excipients, Carriers, and Diluents

In certain embodiments, the platinum-based antineoplastic agent and the LIF-binding polypeptides of the current disclosure are a component of a pharmaceutical composition. In certain embodiments, the platinum-based antineoplastic agent and the LIF-binding polypeptides of the current disclosure are a component of the same pharmaceutical composition. In certain embodiments, the pharmaceutical composition comprises a physiologically appropriate salt concentration (e.g., NaCl). In certain embodiments, the pharmaceutical composition comprises between about 0.6% and 1.2% NaCl. In certain embodiments, the pharmaceutical composition comprises between about 0.7% and 1.1% NaCl. In certain embodiments, the pharmaceutical composition comprises between about 0.8% and 1.0% NaCl. In certain embodiments, the pharmaceutical composition further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20),polysorbate and poloxamer 188; polyol/disaccharide/polysaccharides, for example, glucose, dextrose, mannose, mannitol, sorbitol, sucrose, trehalose, and dextran 40; amino acids, for example, histidine, glycine or arginine; antioxidants, for example, ascorbic acid, methionine; and chelating agents, for example, EGTA or EGTA.

In certain embodiments, the platinum-based antineoplastic agent, the LIF-binding polypeptides, or both the platinum-based antineoplastic agent and the LIF-binding polypeptides of the current disclosure are administered suspended in a sterile solution. In certain embodiments, the platinum-based antineoplastic agent and the LIF-binding polypeptides are administered from the same solution. In certain embodiments, the solution comprises a physiologically appropriate salt concentration (e.g., NaCl). In certain embodiments, the solution comprises between about 0.6% and 1.2% NaCl. In certain embodiments, the solution comprises between about 0.7% and 1.1% NaCl. In certain embodiments, the solution comprises between about 0.8% and 1.0% NaCl. In certain embodiments, a highly concentrated stock solution of antibody may be diluted in about 0.9% NaCl. In certain embodiments, the solution comprises about 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), polysorbate and poloxamer 188; polyol/disaccharide/polysaccharides, for example, glucose, dextrose, mannose, mannitol, sorbitol, sucrose, trehalose, and dextran 40; amino acids, for example, histidine, glycine or arginine; antioxidants, for example, ascorbic acid, methionine; and chelating agents, for example, EGTA or EGTA. In certain embodiments, platinum-based antineoplastic agents and the LIF-binding polypeptides of the current disclosure are shipped/stored lyophilized and reconstituted before administration. In certain embodiments, lyophilized antibody formulations comprise a bulking agent such as, mannitol, sorbitol, sucrose, trehalose, and dextran 40. In a certain embodiment, anti-LIF antibodies of this disclosure can be shipped and stored as a concentrated stock solution to be diluted at the treatment site of use. In certain embodiments, the stock solution comprises about 25 mM histidine, about 6% sucrose, about 0.01% polysorbate, and about 20 mg/mL of anti-LIF antibody. In certain embodiments, the pH of the solution is about 6.0. In certain embodiments, the form administered to an individual is an aqueous solution comprising about 25 mM histidine, about 6% sucrose, about 0.01% polysorbate 80, and about 20 mg/mL of h5D8 antibody. In certain embodiments, the pH of the solution is about 6.0.

In certain embodiments, the platinum-based antineoplastic agent and the LIF-binding polypeptides of the current disclosure are administered suspended in a sterile solution. In certain embodiments, the platinum-based antineoplastic agent and the LIF-binding polypeptides are administered from the same solution. In certain embodiments, the platinum-based antineoplastic agent and the LIF-binding polypeptides are administered from separate solutions. In certain embodiments, the solution comprises a physiologically appropriate salt concentration (e.g., NaCl). In certain embodiments, the solution comprises between about 0.6% and 1.2% NaCl. In certain embodiments, the solution comprises between about 0.7% and 1.1% NaCl. In certain embodiments, the solution comprises between about 0.8% and 1.0% NaCl. In certain embodiments, a highly concentrated stock solution of antibody may be diluted in about 0.9% NaCl. In certain embodiments, the solution comprises about 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20),polysorbate and poloxamer 188; polyol/disaccharide/polysaccharides, for example, glucose, dextrose, mannose, mannitol, sorbitol, sucrose, trehalose, and dextran 40; amino acids, for example, histidine, glycine or arginine; antioxidants, for example, ascorbic acid, methionine; and chelating agents, for example, EGTA or EGTA. In certain embodiments, platinum-based antineoplastic agents and the LIF-binding polypeptides of the current disclosure are shipped/stored lyophilized and reconstituted before administration. In certain embodiments, lyophilized antibody formulations comprise a bulking agent such as, mannitol, sorbitol, sucrose, trehalose, and dextran 40. In a certain embodiment, anti-LIF antibodies of this disclosure can be shipped and stored as a concentrated stock solution to be diluted at the treatment site of use. In certain embodiments, the stock solution comprises about 25 mM histidine, about 6% sucrose, about 0.01% polysorbate, and about 20 mg/mL of anti-LIF antibody. In certain embodiments, the pH of the solution is about 6.0. In certain embodiments, the form administered to an individual is an aqueous solution comprising about 25 mM histidine, about 6% sucrose, about 0.01% polysorbate 80, and about 20 mg/mL of h5D8 antibody. In certain embodiments, the pH of the solution is about 6.0.

Also described herein are kits for carrying out the combination therapies described herein. In certain embodiments, a kit comprises a LIF-binding polypeptide and a platinum-based antineoplastic agent. In certain embodiments, a kit comprises h5D8 and a platinum-based antineoplastic agent. Either or both components can be contained in a vial of glass or other suitable material in either a lyophilized or a liquid form.

In certain embodiments, described herein is use of a LIF-binding polypeptide, in combination with a platinum-based antineoplastic agent, for treating a non-small lung cancer, epithelial ovarian carcinoma, or pancreatic adenocarcinoma in an individual. In certain embodiments, the LIF-binding polypeptide and the platinum-based antineoplastic agent are administered to the individual in separate formulations. In certain embodiments, the LIF-binding polypeptide and the platinum-based antineoplastic agent are administered to the individual in the same formulation. In certain embodiments, the LIF-binding polypeptide is administered to the individual before the platinum-based antineoplastic agent is administered to the individual. In certain embodiments, the platinum-based antineoplastic agent is administered to the individual before the LIF-binding polypeptide is administered to the individual. In certain embodiments, the platinum-based antineoplastic agent is administered to the individual at the same time as the LIF-binding polypeptide is administered to the individual. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the antibody that specifically binds to LIF comprises at least one framework region derived from a human antibody framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, or 99% identical to the amino acid sequence set forth in SEQ ID NO: 42; and the VL sequence is at least about 80%, 90%, 95%, 97%, 98%, or 99% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the cancer is refractory to treatment with a therapeutic amount of a LIF-binding polypeptide or a platinum-based antineoplastic agent administered as a monotherapy.

In certain embodiments, described herein is use of a LIF-binding polypeptide, in combination with a platinum-based antineoplastic agent, for treating a colorectal cancer in an individual. In certain embodiments, the LIF-binding polypeptide and the platinum-based antineoplastic agent are administered to the individual in separate formulations. In certain embodiments, the LIF-binding polypeptide and the platinum-based antineoplastic agent are administered to the individual in the same formulation. In certain embodiments, the LIF-binding polypeptide is administered to the individual before the platinum-based antineoplastic agent is administered to the individual. In certain embodiments, the platinum-based antineoplastic agent is administered to the individual before the LIF-binding polypeptide is administered to the individual. In certain embodiments, the platinum-based antineoplastic agent is administered to the individual at the same time as the LIF-binding polypeptide is administered to the individual. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the antibody that specifically binds to LIF comprises at least one framework region derived from a human antibody framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, or 99% identical to the amino acid sequence set forth in SEQ ID NO: 42; and the VL sequence is at least about 80%, 90%, 95%, 97%, 98%, or 99% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the cancer is refractory to treatment with a therapeutic amount of a LIF-binding polypeptide or a platinum-based antineoplastic agent administered as a monotherapy.

In certain embodiments, described herein is use of a LIF-binding polypeptide, in combination with a platinum-based antineoplastic agent, for treating a glioblastoma multiforme in an individual. In certain embodiments, the LIF-binding polypeptide and the platinum-based antineoplastic agent are administered to the individual in separate formulations. In certain embodiments, the LIF-binding polypeptide and the platinum-based antineoplastic agent are administered to the individual in the same formulation. In certain embodiments, the LIF-binding polypeptide is administered to the individual before the platinum-based antineoplastic agent is administered to the individual. In certain embodiments, the platinum-based antineoplastic agent is administered to the individual before the LIF-binding polypeptide is administered to the individual. In certain embodiments, the platinum-based antineoplastic agent is administered to the individual at the same time as the LIF-binding polypeptide is administered to the individual. In certain embodiments, the LIF-binding polypeptide comprises a fragment of an immunoglobulin variable region, or an immunoglobulin heavy chain constant region. In certain embodiments, the LIF-binding polypeptide comprises an antibody that specifically binds to LIF. In certain embodiments, the antibody that specifically binds to LIF comprises at least one framework region derived from a human antibody framework region. In certain embodiments, the antibody that specifically binds to LIF is humanized. In certain embodiments, the antibody that specifically binds to LIF is deimmunized. In certain embodiments, the antibody that specifically binds to LIF comprises two immunoglobulin heavy chains and two immunoglobulin light chains. In certain embodiments, the antibody that specifically binds to LIF is an IgG antibody. In certain embodiments, the antibody that specifically binds to LIF is a Fab, F(ab)₂, single-domain antibody, a single chain variable fragment (scFv), or a nanobody. In certain embodiments, the antibody that specifically binds to LIF comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the antibody that specifically binds to LIF comprises: (a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and (b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. In certain embodiments, the VH sequence is at least about 80%, 90%, 95%, 97%, 98%, or 99% identical to the amino acid sequence set forth in SEQ ID NO: 42; and the VL sequence is at least about 80%, 90%, 95%, 97%, 98%, or 99% identical to the amino acid sequence set forth in SEQ ID NO: 46. In certain embodiments, the VH sequence is identical to the amino acid sequence set forth in SEQ ID NO: 42; and the VL sequence is identical to the amino acid sequence set forth in SEQ ID NO: 46. In certain embodiments, the antibody that specifically binds to LIF comprises: (a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and (b) an immunoglobulin light chain sequence with the amino acid sequence at least about 80%, 90%, 95%, 97%, 98%, 99%, or 100% identical to the amino acid sequence set forth in any one of SEQ ID NOs: 61-64. In certain embodiments, the antibody that specifically binds to LIF binds with a K_{D} of less than about 200 picomolar. In certain embodiments, the antibody that specifically binds to LIF binds with a K_{D} of less than about 100 picomolar. In certain embodiments, the platinum-based antineoplastic agent comprises cisplatin, carboplatin, oxaliplatin, nedaplatin, triplatin tetranitrate, phenathriplatin, picoplatin, satraplatin, or combinations thereof. In certain embodiments, the cancer is refractory to treatment with a therapeutic amount of a LIF-binding polypeptide or a platinum-based antineoplastic agent administered as a monotherapy.

As used herein “treating” or “treatment” refers to the intervention in a disease state intended to produce one or more beneficial effects. For cancer/tumor purposes treatment includes methods that are intended to cause or do cause stable disease, partial response, complete response, extension of progression-free survival, extension of overall survival, tumor shrinkage, a delay in tumor growth, an arrest of tumor growth, or a prevention or reduction in metastasis. In certain cases the therapeutic methods described herein may be used as maintenance after successful treatment or to prevent recurrence or metastasis of a particular tumor or cancer. It is understood that not all individuals will respond to the same degree, or at all, to a given administration of therapeutic antibody, however even if no response is detected these individuals are nonetheless considered to have been treated.

### Exemplary Embodiments

Among the exemplary embodiments are: 1. Use of a Leukemia Inhibitory Factor (LIF)-binding polypeptide, in combination with a platinum-based antineoplastic agent, for treating a cancer in an individual. 2. The use of embodiment 1, wherein the LIF-binding polypeptide and the platinum-based antineoplastic agent are administered to the individual in separate formulations. 3. The use of embodiment 1, wherein the LIF-binding polypeptide and the platinum-based antineoplastic agent are administered to the individual in the same formulation. 4. The use of embodiment 1 or 2, wherein the LIF-binding polypeptide is administered to the individual before the platinum-based antineoplastic agent is administered to the individual. 5. The use of embodiment 1 or 2, wherein the platinum-based antineoplastic agent is administered to the individual before the LIF-binding polypeptide is administered to the individual. 6. The use of any one of embodiments 1 to 3, wherein the platinum-based antineoplastic agent is administered to the individual at the same time as the LIF-binding polypeptide is administered to the individual. 7. The use of any one of embodiments 1 to 6, wherein the LIF-binding polypeptide comprises a fragment of an immunoglobulin variable region, or an immunoglobulin heavy chain constant region. 8. The use of embodiment 7, wherein the LIF-binding polypeptide comprises an antibody that specifically binds to LIF. 9. The use of embodiment 8, wherein the antibody that specifically binds to LIF comprises at least one framework region derived from a human antibody framework region. 10. The use of embodiment 8, wherein the antibody that specifically binds to LIF is humanized. 11. The use of embodiment 8, wherein the antibody that specifically binds to LIF is deimmunized. 12. The use of embodiment 8, wherein the antibody that specifically binds to LIF comprises two immunoglobulin heavy chains and two immunoglobulin light chains. 13. The use of embodiment 8, wherein the antibody that specifically binds to LIF is an IgG antibody. 14. The use of embodiment 8, wherein the antibody that specifically binds to LIF is a Fab, F(ab)2, single-domain antibody, a single chain variable fragment (scFv), or a nanobody. 15. The use of any one of embodiments 8 to 14, wherein the antibody that specifically binds to LIF comprises: a) an immunoglobulin heavy chain complementarity determining region 1 (VH-CDR1) comprising the amino acid sequence set forth in any one of SEQ ID NOs: 1-3; b) an immunoglobulin heavy chain complementarity determining region 2 (VH-CDR2) comprising the amino acid sequence set forth in any one of SEQ ID NOs: 4 or 5; c) an immunoglobulin heavy chain complementarity determining region 3 (VH-CDR3) comprising the amino acid sequence set forth in any one of SEQ ID NOs: 6-8; d) an immunoglobulin light chain complementarity determining region 1 (VL-CDR1) comprising the amino acid sequence set forth in any one of SEQ ID NOs: 9 or 10; e) an immunoglobulin light chain complementarity determining region 2 (VL-CDR2) comprising the amino acid sequence set forth in any one of SEQ ID NOs: 11 or 12; and f) an immunoglobulin light chain complementarity determining region 3 (VL-CDR3) comprising the amino acid sequence set forth in SEQ ID NO: 13. 16. The use of any one of embodiments 8 to 15, wherein the antibody that specifically binds to LIF comprises: a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. 17. The use of embodiment 16, wherein the VH sequence is at least about 80%, 90%, 95%, 97%, 98%, or 99% identical to the amino acid sequence set forth in SEQ ID NO: 42; and the VL sequence is at least about 80%, 90%, 95%, 97%, 98%, or 99% identical to the amino acid sequence set forth in SEQ ID NO: 46. 18. The use of embodiment 17, wherein the VH sequence is identical to the amino acid sequence set forth in SEQ ID NO: 42; and the VL sequence is identical to the amino acid sequence set forth in SEQ ID NO: 46. 19. The use of any one of embodiments 8 to 18, wherein the antibody that specifically binds to LIF comprises: a) an immunoglobulin heavy chain sequence with the amino acid sequence at least about 80%, 90%, 95%, 97%, 98%, 99%, or 100% identical to the amino acid sequence set forth in any one of SEQ ID NOs: 57-60 or 67; and b) an immunoglobulin light chain sequence with the amino acid sequence at least about 80%, 90%, 95%, 97%, 98%, 99%, or 100% identical to the amino acid sequence set forth in any one of SEQ ID NOs: 61-64. 20. The use of any one of embodiments 8 to 19, wherein the antibody that specifically binds to LIF binds with a KD of less than about 200 picomolar. 21. The use of any one of embodiments 8 to 19, wherein the antibody that specifically binds to LIF binds with a KD of less than about 100 picomolar. 22. The use of any one of embodiments 10 to 21, wherein the platinum-based antineoplastic agent comprises cisplatin, carboplatin, oxaliplatin, nedaplatin, triplatin tetranitrate, phenathriplatin, picoplatin, satraplatin, or combinations thereof. 23. The use of embodiment 22, wherein the platinum-based antineoplastic agent is cisplatin. 24. The use of any one of embodiments 1 to 23, wherein the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. 25. The use of embodiment 24, wherein the cancer comprises non-small cell lung cancer, epithelial ovarian carcinoma, or pancreatic adenocarcinoma. 26. The use of any one of embodiments 1 to 25, wherein the cancer is refractory to treatment with a therapeutic amount of a LIF-binding polypeptide or a platinum-based antineoplastic agent administered as a monotherapy. 27. A method of treating an individual with a cancer, comprising administering to the individual with the cancer an effective amount of a combination of: a) a Leukemia Inhibitory Factor (LIF) binding polypeptide; and b) a platinum-based antineoplastic agent. 28. The method of embodiment 27, comprising administering an effective amount of the LIF-binding polypeptide to the individual with cancer. 29. The method of embodiment 27, comprising administering an effective amount of the platinum-based antineoplastic agent to the individual with cancer. 30. The method of any one of embodiments 27 to 29, wherein the LIF-binding polypeptide comprises a fragment of an immunoglobulin variable region, or an immunoglobulin heavy chain constant region. 31. The method of any one of embodiments 27 to 29, wherein the LIF-binding polypeptide comprises an antibody that specifically binds to LIF. 32. The method of embodiment 31, wherein the antibody that specifically binds to LIF comprises at least one framework region derived from a human antibody framework region. 33. The method of embodiment 31, wherein the antibody that specifically binds to LIF is humanized. 34. The method of embodiment 31, wherein the antibody that specifically binds to LIF is deimmunized. 35. The method of embodiment 31, wherein the antibody that specifically binds to LIF comprises two immunoglobulin heavy chains and two immunoglobulin light chains. 36. The method of embodiment 31, wherein the antibody that specifically binds to LIF is an IgG antibody. 37. The method of embodiment 31, wherein the antibody that specifically binds to LIF is a Fab, F(ab)2, single-domain antibody, a single chain variable fragment (scFv), or a nanobody. 38. The method of any one of embodiments 31 to 37, wherein the antibody that specifically binds to LIF comprises: a) an immunoglobulin heavy chain complementarity determining region 1 (VH-CDR1) comprising the amino acid sequence set forth in any one of SEQ ID NOs: 1-3; b) an immunoglobulin heavy chain complementarity determining region 2 (VH-CDR2) comprising the amino acid sequence set forth in any one of SEQ ID NOs: 4 or 5; c) an immunoglobulin heavy chain complementarity determining region 3 (VH-CDR3) comprising the amino acid sequence set forth in any one of SEQ ID NOs: 6-8; d) an immunoglobulin light chain complementarity determining region 1 (VL-CDR1) comprising the amino acid sequence set forth in any one of SEQ ID NOs: 9 or 10; e) an immunoglobulin light chain complementarity determining region 2 (VL-CDR2) comprising the amino acid sequence set forth in any one of SEQ ID NOs: 11 or 12; and f) an immunoglobulin light chain complementarity determining region 3 (VL-CDR3) comprising the amino acid sequence set forth in SEQ ID NO: 13. 39. The method of any one of embodiments 31 to 37, wherein the antibody that specifically binds to LIF comprises: a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48. 40. The method of embodiment 39, wherein the VH sequence is at least about 80%, 90%, 95%, 97%, 98%, 99%, or 100% identical to the amino acid sequence set forth in SEQ ID NO: 42; and the VL sequence is at least about 80%, 90%, 95%, 97%, 98%, 99%, or 100% identical to the amino acid sequence set forth in SEQ ID NO: 46. 41. The method of embodiment 40, wherein the VH sequence is identical to the amino acid sequence set forth in SEQ ID NO: 42; and the VL sequence is identical to the amino acid sequence set forth in SEQ ID NO: 46. 42. The method of any one of embodiments 31 to 37, wherein the antibody that specifically binds to LIF comprises: a) an immunoglobulin heavy chain sequence with the amino acid sequence at least about 80%, 90%, 95%, 97%, 98%, or 99% identical to the amino acid sequence set forth in any one of SEQ ID NOs: 57-60 or 67; and b) an immunoglobulin light chain sequence with the amino acid sequence at least about 80%, 90%, 95%, 97%, 98%, or 99% identical to the amino acid sequence set forth in any one of SEQ ID NOs: 61-64. 43. The method of any one of embodiments 31 to 42, wherein the antibody that specifically binds to LIF binds with a KD of less than about 200 picomolar. 44. The method of any one of embodiments 31 to 42, wherein the antibody that specifically binds to LIF binds with a KD of less than about 100 picomolar. 45. The method of any one of embodiments 27 to 44, wherein the platinum-based antineoplastic agent comprises cisplatin, carboplatin, oxaliplatin, nedaplatin, triplatin tetranitrate, phenathriplatin, picoplatin, satraplatin, or combinations thereof. 46. The method of embodiment 45, wherein the platinum-based antineoplastic agent is cisplatin. 47. The method of any one of embodiments 27 to 46, wherein the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. 48. The method of embodiment 47, wherein the cancer comprises non-small cell lung cancer, epithelial ovarian carcinoma, or pancreatic adenocarcinoma. 49. The method of any one of embodiments 27 to 48, wherein the cancer is refractory to treatment with a therapeutic amount of an inhibitor of a LIF-binding polypeptide. 50. The method of any one of embodiments 27 to 48, wherein the cancer is refractory to treatment with a therapeutic amount of a platinum-based antineoplastic agent. 51. The method of any one of embodiments 27 to 50, wherein the Leukemia Inhibitory Factor (LIF) binding polypeptide and the platinum-based antineoplastic agent are administered separately. 52. The method of any one of embodiments 27 to 50, wherein the LIF-binding polypeptide and the platinum-based antineoplastic agent are administered at the same time. 53. The method of any one of embodiments 27 to 50, wherein the LIF-binding polypeptide and the platinum-based antineoplastic agent are administered in a single composition. 54. Use of an antibody that specifically binds Leukemia Inhibitory Factor (LIF), in combination with a platinum-based antineoplastic agent, for treating a cancer in an individual, wherein the LIF binding antibody comprises: a) an immunoglobulin heavy chain complementarity determining region 1 (VH-CDR1) comprising the amino acid sequence set forth in any one of SEQ ID NOs: 1-3; b) an immunoglobulin heavy chain complementarity determining region 2 (VH-CDR2) comprising the amino acid sequence set forth in any one of SEQ ID NOs: 4 or 5; c) an immunoglobulin heavy chain complementarity determining region 3 (VH-CDR3) comprising the amino acid sequence set forth in any one of SEQ ID NOs: 6-8; d) an immunoglobulin light chain complementarity determining region 1 (VL-CDR1) comprising the amino acid sequence set forth in any one of SEQ ID NOs: 9 or 10; e) an immunoglobulin light chain complementarity determining region 2 (VL-CDR2) comprising the amino acid sequence set forth in any one of SEQ ID NOs: 11 or 12; and f) an immunoglobulin light chain complementarity determining region 3 (VL-CDR3) comprising the amino acid sequence set forth in SEQ ID NO: 13. 55. A method of treating an individual with a cancer comprising administering to the individual with cancer an effective amount of a combination of: a) of an antibody that specifically binds Leukemia Inhibitory Factor (LIF) comprising: i. an immunoglobulin heavy chain complementarity determining region 1 (VH-CDR1) comprising the amino acid sequence set forth in any one of SEQ ID NOs: 1-3; ii. an immunoglobulin heavy chain complementarity determining region 2 (VH-CDR2) comprising the amino acid sequence set forth in any one of SEQ ID NOs: 4 or 5; iii. an immunoglobulin heavy chain complementarity determining region 3 (VH-CDR3) comprising the amino acid sequence set forth in any one of SEQ ID NOs: 6-8; iv. an immunoglobulin light chain complementarity determining region 1 (VL-CDR1) comprising the amino acid sequence set forth in any one of SEQ ID NOs: 9 or 10; v. an immunoglobulin light chain complementarity determining region 2 (VL-CDR2) comprising the amino acid sequence set forth in any one of SEQ ID NOs: 11 or 12; and vi. an immunoglobulin light chain complementarity determining region 3 (VL-CDR3) comprising the amino acid sequence set forth in SEQ ID NO: 13; and b) a platinum-based antineoplastic agent. 56. The method of embodiment 55, comprising administering an effective amount of the LIF-binding polypeptide to the individual with cancer. 57. The method of embodiment 55, comprising administering an effective amount of the platinum-based antineoplastic agent to the individual with cancer.

### EXAMPLES

The following illustrative examples are representative of embodiments of the compositions and methods described herein and are not meant to be limiting in any way.

### Example 1

### Generation of Rat Antibodies Specific for LIF

A cDNA encoding amino acids 23-202 of human LIF was cloned into expression plasmids (Aldevron GmbH, Freiburg, Germany). Groups of laboratory rats (Wistar) were immunized by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the LIF protein. Serum samples were collected after a series of immunizations and tested in flow cytometry on HEK cells transiently transfected with the aforementioned expression plasmids. Antibody-producing cells were isolated and fused with mouse myeloma cells (Ag8) according to standard procedures. Hybridomas producing antibodies specific for LIF were identified by screening in a flow cytometry assay as described above. Cell pellets of positive hybridoma cells were prepared using an RNA protection agent (RNAlater, cat. #AM7020 by ThermoFisher Scientific) and further processed for sequencing of the variable domains of the antibodies.

### Example 2

### Generation of Mouse Antibodies Specific for LIF

A cDNA encoding amino acids 23-202 of human LIF was cloned into expression plasmids (Aldevron GmbH, Freiburg, Germany). Groups of laboratory mice (NMRI) were immunized by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the LIF protein. Serum samples were collected after a series of immunizations and tested in flow cytometry on HEK cells transiently transfected with the aforementioned expression plasmids. Antibody-producing cells were isolated and fused with mouse myeloma cells (Ag8) according to standard procedures. Hybridomas producing antibodies specific for LIF were identified by screening in a flow cytometry assay as described above. Cell pellets of positive hybridoma cells were prepared using an RNA protection agent (RNAlater, cat. #AM7020 by ThermoFisher Scientific) and further processed for sequencing of the variable domains of the antibodies.

### Example 3

### Humanization of Rat Antibodies Specific for LIF

One clone from the rat immunization (5D8) was chosen for subsequent humanization. Humanization was conducted using standard CDR grafting methods. The heavy chain and light chain regions were cloned from the 5D8 hybridoma using standard molecular cloning techniques and sequenced by the Sanger method. A BLAST search was then conducted against human heavy chain and light chain variable sequences and 4 sequences from each were chosen as acceptor frameworks for humanization. These acceptor frameworks were deimmunized to remove T cell response epitopes. The heavy chain and light chain CDR1, CDR2 and CDR3 of 5D8 were cloned into the 4 different heavy chain acceptor frameworks (H1 to H4), and 4 different light chain frameworks (L1 to L4). Then all 16 different antibodies were tested for: expression in CHO-S cells (Selexis); inhibition of LIF-induced STAT3 phosphorylation; and binding affinity by Surface Plasmon Resonance (SPR). These experiments are summarized in

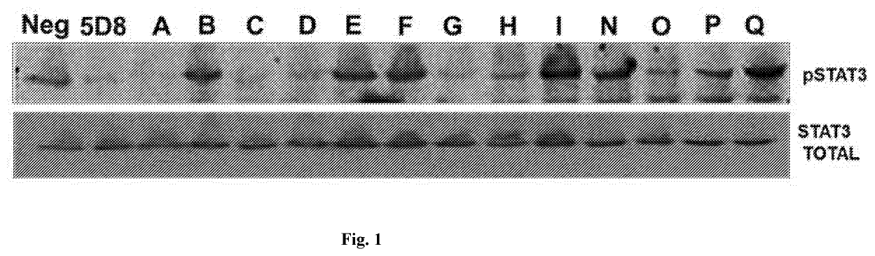

TABLE 1
Summary of 5D8 humanization

| Heavy chain | Inhibition of | Affinity by | |
|---|---|---|---|
| light chain | LIF-induced | SPR | Expression |
| combination | pSTAT3 from FIG. 1 | K_{D1} (pM) | (ug/mL) |
| H0L0 | +++ | 133 ± 46 | 393 |
| H1L1 | − | N/A | 627 |
| H1L2 | +++ | 55 ± 23 | 260 |
| H1L3 | +++ | 54 ± 31 | 70 |
| H1L4 | − | N/A | 560 |
| H2L1 | − | N/A | 369 |
| H2L2 | +++ | 52 ± 22 | 392 |
| H2L3 | ++ | 136 ± 19 | 185 |
| H2L4 | − | N/A | 78 |
| H3L1 | N/A | N/A | No expression |
| H3L2 | N/A | N/A | No expression |
| H3L3 | N/A | N/A | No expression |
| H3L4 | N/A | N/A | No expression |
| H4L1 | − | N/A | 259 |
| H4L2 | ++ | 913 ± 308 | 308 |
| H4L3 | + | 252 | |
| H4L4 | − | N/A | 186 |

N/A = Not attempted; H0L0 = chimeric antibody with full rat heavy and light chain variable regions

The expression performance of the transfected cells was compared in Erlenmeyer flasks (seeding 3×10⁵ cells/mL, 200 mL culture volume) within fed-batch cultivation after 10 days of cell culture. At this point cells were harvested and the secreted antibody purified using a Protein A column and then quantitated. All humanized antibodies expressed except those using the H3 heavy chain (SEQ ID NO: 43).

Inhibition of LIF-induced STAT3 phosphorylation at tyrosine 705 was determined by western blot. U251 glioma cells were plated in 6-well plates at a density of 100.000 cells/well. Cells were cultured in complete medium for 24 hours before any treatment and after that, cells were serum starved for 8 hours. After that, cells with the indicated antibodies over night at a concentration of 10 μg/ml. After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fat dried milk—TBST and incubated with the primary antibody overnight (p-STAT3, catalog #9145, Cell Signaling or STAT3, catalog #9132, Cell Signaling) or 30 minutes (β-actin-peroxidase, catalog #A3854, Sigma-Aldrich). Membranes were then washed with TBST, incubated with secondary and washed again. Proteins were detected by chemiluminescence (SuperSignal Substrate, catalog #34076, Thermo Fisher Scientific). These results are shown in FIG. 1. The darker the pSTAT3 band the less inhibition is present. Inhibition was high in lanes labeled 5D8 (non humanized rat), A(H0L0), C (H1L2), D (H1L3), and G (H2L2); inhibition was moderate in H (H2L3), O (H4L2), and P (H4L3); inhibition was absent in B (H1L1), E (H1L4), F (H2L1), I (H2L4), N (H4L1) and Q (H4L4).

Antibodies that exhibited inhibition of LIF-induced STAT3 phosphorylation were then analyzed by SPR to determine binding affinity. Briefly, binding of the A(H0L0), C (H1L2), D (H1L3), and G (H2L2), H (H2L3) and O (H4L2) humanized antibodies to amine coupled hLIF was observed using a Biacore™ 2002 Instrument. Kinetic constants and affinities were determined by mathematical sensorgram fitting (Langmuir interaction model [A+B=AB]) of all sensorgrams generated on all sensor chip surfaces at six ligand concentrations. The best fitted curves (minimal Chi2) of each concentration were used for calculation of kinetic constants and affinities. See Table 1.

Since the experimental setup used bivalent antibodies as analytes, best fitted sensorgrams, were also analyzed on basis of a bivalent analyte fitting model [A+B=AB; AB+B=AB2] in order to obtain a more detailed insight into the target binding mechanism of the humanized antibodies. Kinetic sensorgram analysis using a bivalent fitting model [A+B=AB; AB+B=AB2] confirmed the relative affinity ranking of the mAb samples.

The humanized 5D8 comprising H2 and L2 was selected for more in-depth analysis due to its high binding affinity and high yield from batch culture.

### Example 4

### Humanization of Clone 5D8 Improves Binding to LIF

The H2L2 clone (h5D8) was selected for further analysis and compared binding by SPR to the parental rat 5D8 (r5D8) and a mouse clone 1B2. The 1B2 antibody is a previously disclosed mouse anti-LIF antibody previously deposited at the Deutsche Sammlung von Mikroorganismen and Zellkulturen GmbH (DSM ACC3054) and was included for comparison purposes. Recombinant human LIF, purified from E.coli and HEK-293 cells, respectively, were used as ligands. The LIF from human or E. coli sources was covalently coupled to the surface of Biacore optical sensor chips using amine coupling chemistry, and binding affinities were calculated from the kinetic constants.

### Materials and Methods

Human LIF from E.coli was obtained from Millipore, reference LIF 1010; human LIF from HEK-293 cells was obtained from ACRO Biosystems, reference LIF-H521b. LIF was coupled to the sensor chips using the Biacore Amine Coupling Kit (BR-1000-50; GE-Healthcare, Uppsala). Samples were run on a Biacore™ 2002 Instrument using CM5 optical sensor chips (BR-1000-12; GE-Healthcare, Uppsala). Biacore HBS-EP buffer was used during the machine runs (BR-1001-88; GE-Healthcare, Uppsala). Kinetic analysis of binding sensorgrams was performed using BIAevaluation 4.1 software. Kinetic constants and affinities were determined by mathematical sensorgram fitting (Langmuir interaction model [A+B=AB]) of all sensorgrams generated on all sensor chip surfaces at increasing analyte concentrations. Sensorgrams were also analyzed on the basis of a bivalent analyte sensorgram fitting model [A+B=AB; AB+B=AB₂], including component analysis, in order to generate an estimate on the bivalent contribution to the determined Langmuir antibody—target affinities (e.g., avidity contribution). The best fitted curves (minimal Chi²) of each concentration were used for calculation of kinetic constants and affinities. Summaries of these affinity experiments are shown in Table 2 (human LIF made in E.coli) and Table 3 (human LIF made in HEK 293 cells).

TABLE 2

| Improved binding of | K_{D} [pM] | |
|---|---|---|
| 5D8 after humanization | Langmuir 1:1 | Bivalent |
| hLIF (E. coli) | sensorgram fitting | analyte fitting |
| Mouse 1B2 | 400 ± 210 | 1500 ± 200 |
| r5D8 (Rat) | 130 ± 30 | 780 ± 130 |
| h5D8 (humanized) | 26 ± 14 | 82 ± 25 |

TABLE 3

| Improved binding of | K_{D} [pM] | |
|---|---|---|
| 5D8 after humanization | Langmuir 1:1 | Bivalent analyte |
| hLIF (HEK 293) | sensorgram fitting | fitting |
| Mouse 1B2 | 320 ± 150 | 3900 ± 900 |
| r5D8 (rat) | 135 ± 100 | 410 ± 360 |
| h5D8 (humanized) | 13 ± 6 | 63 ± 30 |

The Langmuir 1:1 sensorgram fitting model from this set of experiments indicates that the humanized 5D8 (h5D8) antibody bound with ˜10-25 times higher affinity to human LIF than mouse 1B2 and r5D8.

Next, the h5D8 antibody was tested against LIF of multiple species by SPR. h5D8 SPR binding kinetics were performed for recombinant LIF analytes derived from different species and expression systems: human LIF (E.coli, HEK293 cells); mouse LIF (E.coli, CHO cells); rat LIF (E.coli); cynomolgus monkey LIF (yeast, HEK293 cells).

### Materials and Methods

The h5D8 antibody was immobilized to the sensor chip surface by non-covalent, Fc specific capturing. Recombinant, Ig(Fc) specific S. aureus Protein A/G was used as capturing agent, allowing sterically uniform and flexible presentation of the anti-LIF antibody to the LIF analytes. Sources of the LIF analytes are as follows: Human LIF (from E.coli; Millipore reference LIF 1050); Human LIF (from HEK cells ACRO Biosystems LIF-H521); Mouse LIF (E. coli; Millipore Cat. No NF-LIF2010); Mouse LIF (from CHO cells; Reprokine Catalog #RCP09056); Monkey LIF (yeast Kingfisher Biotech Catalog #RP1074Y); Monkey LIF produced in HEK-293 cell. Overall h5D8 exhibited binding to LIF from several species. A summary of this affinity experiment is shown in Table 4.

TABLE 4

| Broad species | Langmuir 1:1 sensorgram fitting | | |
|---|---|---|---|
| reactivity of humanized 5D8 | mean Kₐ | mean K_{d} | mean |
| Analyte | (1/Ms)[10⁵] | (1/S) [10⁻⁵] | K_{D} [pM] |
| Human LIF (E. coli) | 8.5 ± 0.7 | 7.2 ± 0.7 | 86 ± 9 |
| Human LIF (HEK-293) | 5.5 ± 0.02 | 3.1 ± 0.7 | 56 ± 13 |
| Mouse LIF (E. coli) | 21.4 ± 3.7 | 5.7 ± 1.0 | 27 ± 6 |
| Mouse LIF(CHO cells) | 6.5 ± 0.7 | 1.1 ± 0.3 | 17 ± 4 |
| Cyno Monkey LIF (yeast) | 6.3 ± 0.8 | 5.4 ± 0.7 | 89 ± 10 |
| Cyno Monkey LIF (HEK-293) | 2.4 ± 0.2 | 3.3 ± 0.3 | 134 ± 6 |

### Example 5

### Humanized Clone 5D8 Inhibits LIF-Induced Phosphorylation of STAT3 In Vitro

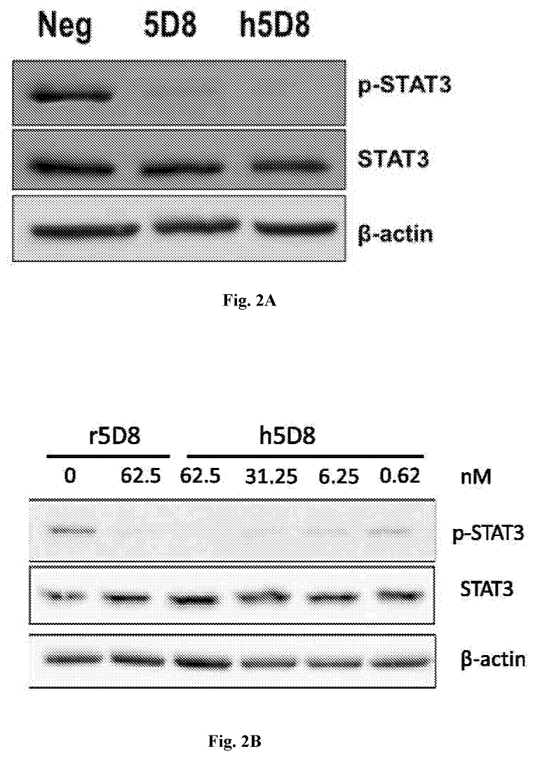

To determine the biological activity of h5D8, the humanized and parental versions were tested in a cell culture model of LIF activation. FIG. 2A shows that the humanized clone exhibited increased inhibition of STAT3 phosphorylation (Tyr 705) when a glioma cell line was incubated with human LIF. FIG. 2B shows an experiment with the same set up of FIG. 2A repeated with different dilutions of the h5D8 antibody.

### Methods

U251 glioma cells were plated in 6-well plates at a density of 150,000 cells/well. Cells were cultured in complete medium for 24 hours before any treatment. After that, cells were treated over night or not (control cells) with r5D8 anti-LIF antibody or h5D8 anti-LIF antibody at a concentration of 10 μg/ml.

After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fatty milk—TBST and incubated with the primary antibody overnight (p-STAT3, catalog #9145, Cell Signaling or STAT3, catalog #9132, Cell Signaling) or 30 minutes (β-actin-peroxidase, catalog #A3854, Sigma-Aldrich). Membranes were then washed with TBST, incubated with secondary antibody if necessary, and washed again. Proteins were detected by chemiluminescence (SuperSignal Substrate, catalog #34076, Thermo Fisher Scientific).

### Example 6

### IC₅₀ Value of h5D8 Antibody Treatment on Endogenous Levels of LIF in U-251 Cells

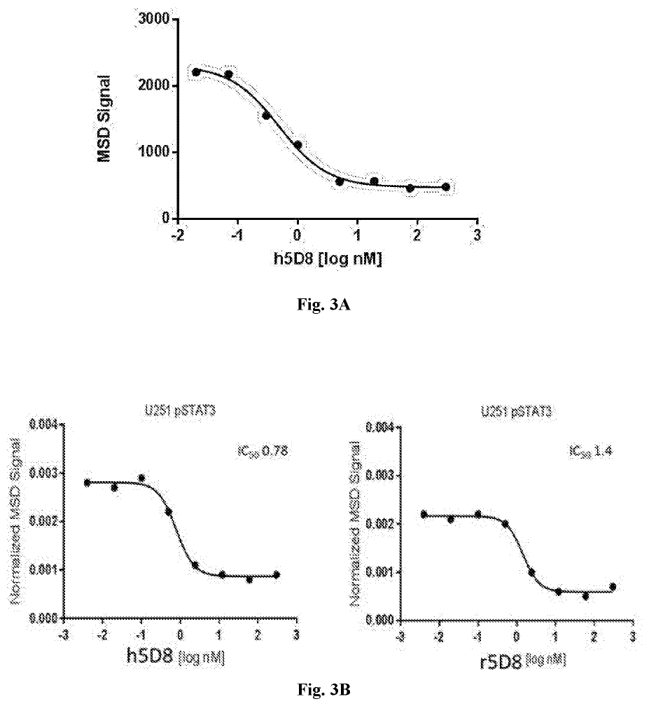

It was also determined that an IC₅₀ of as low as 490 picomolar (FIG. 3A) for biological inhibition for h5D8 under serum starved conditions in U-251 cells. See representative results FIGS. 3A and 3B and Table 5.

TABLE 5

| Cell Line | Cell Line | | | | | | | JAK inhibition |
|---|---|---|---|---|---|---|---|---|
| Tissue | Name | Treatment | IC₅₀ (nM) | | | | IC₉₀ (nM) | (%) |
| Endogenous LIF Condition | | | n = 1 | n = 2 | Mean | SD | Mean | Mean |
| GBM | U251 | h5D8 | 0.78 | 0.54 | 0.66 | 0.12 | 4.1 | 84% |
| | | r5D8 | 1.6 | 1.5 | 1.4 | 0.15 | 8.5 | 86% |
| | | | 1.2 | 1.4 | | | | |

### Methods

The U-251 cells were seeded at 600,000 cells per 6 cm plate (per condition). Cells were treated with h5D8 in corresponding concentration (titration) overnight at 37° C., under serum starvation (0.1% FBS). As a positive control for pSTAT3, recombinant LIF (R&D #7734-LF/CF) was used to stimulate the cells at 1.79 nM for 10 min at 37° C. As a negative control of pSTAT3, the JAK I inhibitor (Calbiochem #420099) was used at 1 uM for 30 min at 37° C. Cells were then harvested on ice for lysates following the Meso Scale Discovery Multi-Spot Assay System Total STAT3 (Cat #K150SND-2) and Phospho-STAT3 (Tyr705) (Cat #K150SVD-2) kits' protocol, to measure protein levels detectable by the MSD Meso Sector S600.

### Example 7

### Additonal Antibodies that Specifically Bind to Human LIF

Other rat antibody clones (10G7 and 6B5) that specifically bind human LIF were identified and a summary of their binding characteristics are shown below in Table 6, clone 1B2 served as a comparison.

### Methods

Kinetic real time binding analysis was performed for anti-LIF mAbs 1B2, 10G7 and 6B5, immobilized on the surface of CM5 optical sensor chips, applying recombinant LIF target proteins [human LIF (E.coli); Millipore Cat. No. LIF 1010 and human LIF (HEK293 cells); ACRO Biosystems Cat. No. LIF-H521b] as analytes.

Kinetic constants and affinities were obtained by mathematical sensorgram fitting using a Langmuir 1:1 binding model applying global (simultaneous fitting of sensorgram sets) as well as single curve fitting algorithms. Plausibility of global fits was assessed by k_{obs} analysis.

TABLE 6

| Affinity measurements | | | | |
|---|---|---|---|---|
| of additional | | Langmuir 1:1 sensorgram fitting | | |
| anti-LIF antibodies | | mean Kₐ | mean K_{d} | mean |
| Analyte | clone | (1/Ms) | (1/S) | K_{D} [nM] |
| Human LIF | 1B2 | 1.1 ± 0.4E5 | 1.1 ± 0.3E−3 | 9.7 ± 1.4 |
| (E. coli) | | | | |
| Human LIF | 1B2 | 2.0 ± 0.04E6 | 1.4 ± 0.2E−3 | 0.7 ± 0.03 |
| (HEK-293) | | | | |
| Human LIF | 10G7 | 7.9 ± 5.8E4 | 6.0 ± 2.3E−4 | 12.6 ± 9.5 |
| (E. coli) | | | | |
| Human LIF | 10G7 | 3.6 ± 1.75E5 | 3.1 ± 0.5E−4 | 1.1 ± 0.6 |
| (HEK-293) | | | | |
| Human LIF | 6B5 | N/A | N/A | N/A |
| (E. coli) | | | | |
| Human LIF | 6B5 | 3.6 ± 1.7E5 | 3.1 ± 0.5E−4 | 62 ± 6 |
| (HEK-293) | | | | |

### Example 8

### Additional Anti LIF Antibodies Inhibit LIF-Induced Phosphorylation of STAT3 In Vitro

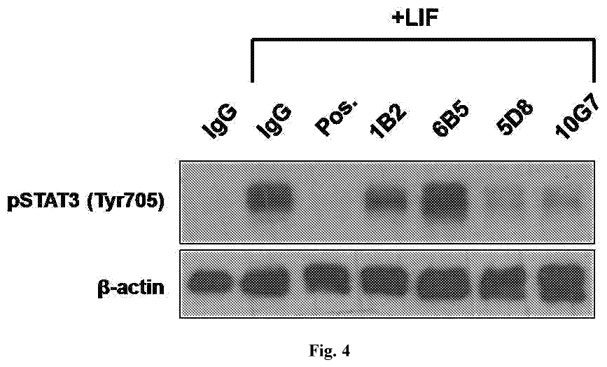

Additional clones were tested for their ability to inhibit LIF-induced phosphorylation of STAT3 in cell culture. As shown in FIG. 4 clones 10G7 and the previously detailed r5D8 exhibited high inhibition of LIF-induced STAT3 phosphorylation, compared to the 1B2 clone. Anti-LIF polyclonal anti-sera (pos.) was included as a positive control While 6B5 exhibited no inhibition, this may be explained by a possible lack of 6B5 binding to non-glycosylated LIF which was used in this experiment.

### Methods

Patient derived glioma cells were plated in 6-well plates at a density of 150,000 cells/well. Cells were cultured in GBM medium that consisted of Neurobasal medium (Life Technologies) supplemented with B27 (Life Technologies), penicillin/streptomycin and growth factors (20 ng/ml EGF and 20 ng/ml FGF-2 [PeproTech]) for 24 hours before any treatment. The following day, cells were treated or not with recombinant LIF produced in E. coli or a mix of recombinant LIF plus the indicated antibodies for 15 minutes (final concentration of 10 μg/ml for the antibodies and 20 ng/ml of recombinant LIF). After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fatty milk—TBST and incubated with the primary antibody overnight (p-STAT3, catalog #9145, Cell Signaling) or 30 minutes (β-actin-peroxidase, catalog #A3854, Sigma-Aldrich). Membranes were then washed with TBST, incubated with secondary antibody if necessary, and washed again. Proteins were detected by chemiluminescence (SuperSignal Substrate, catalog #34076, Thermo Fisher Scientific).

### Example 9

### LIF is Highly Overexpressed Across Multiple Tumor Types

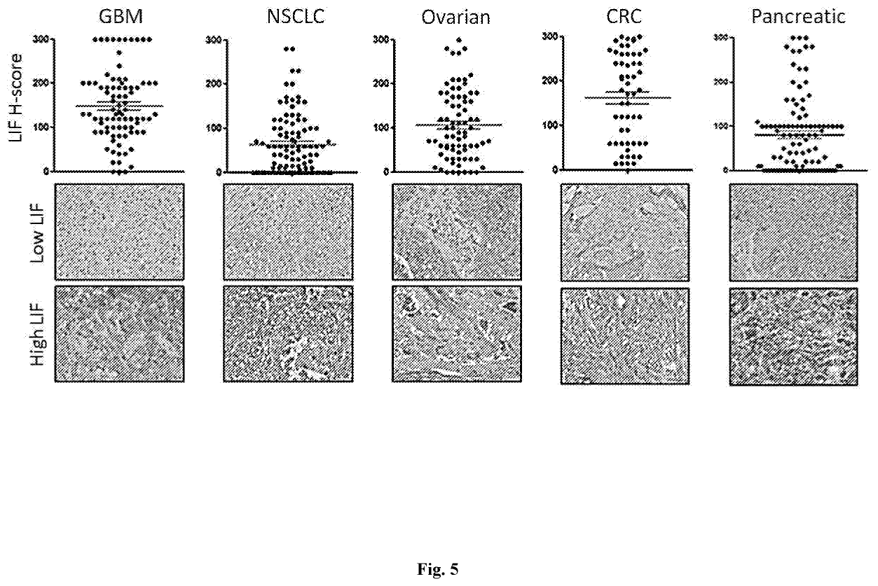

Immunohistochemistry was conducted on multiple human tumor types to determine the degree of LIF expression. As shown in FIG. 5 LIF is highly expressed in glioblastoma multiforme (GBM), non-small cell lung cancer (NSCLC), ovarian cancer, colorectal cancer (CRC), and pancreatic cancer.

### Example 10

### Humanized Clone h5D8 Inhibits Tumor Growth in a Mouse Model of Non-Small Cell Lung Carcinoma

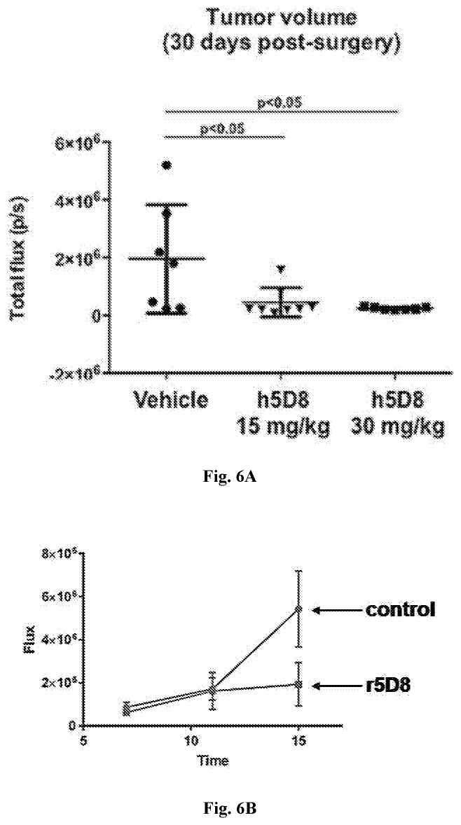

To determine the ability of the humanized 5D8 clone to inhibit a LIF positive cancer in vivo this antibody was tested in a mouse model of non-small cell lung carcinoma (NSCLC). FIG. 6A shows reduced tumor growth in mice treated with this antibody compared to a vehicle negative control. FIG. 6B shows data generated using the r5D8 version.

### Methods

The murine non-small cell lung cancer (NSCLC) cell line KLN205 with high LIF levels was stably infected with lentivirus expressing the firefly luciferase gene for in vivo bioluminescence monitoring. To develop the mouse model, 5×10⁵KLN205 non-small cell lung cancer (NSCLC) cells were orthotopically implanted into the left lung of 8-week-old immunocompetent syngeneic DBA/2 mice by intercostal puncture. Mice were treated with a control vehicle or with 15 mg/kg or 30 mg/kg of the h5D8 antibody intraperitoneally twice a week and tumor growth was monitored by bioluminescence. For the bioluminescence imaging, mice received an intraperitoneal injection of 0.2 mL of 15 mg/mL D-luciferin under 1-2% inhaled isoflurane anesthesia. The bioluminescence signals were monitored using the IVIS system 2000 series (Xenogen Corp., Alameda, Calif., USA) consisting of a highly sensitive cooled CCD camera. Living Image software (Xenogen Corp.) was used to grid the imaging data and integrate the total bioluminescence signals in each boxed region. Data were analyzed using the total photon flux emission (photons/second) in the regions of interest (ROI). The results demonstrate that treatment with the h5D8 antibody promote tumor regression. Data are presented as mean±SEM.

### Example 11

### h5D8 Inhibits Tumor Growth in a Mouse Model of Glioblastoma Multiforme

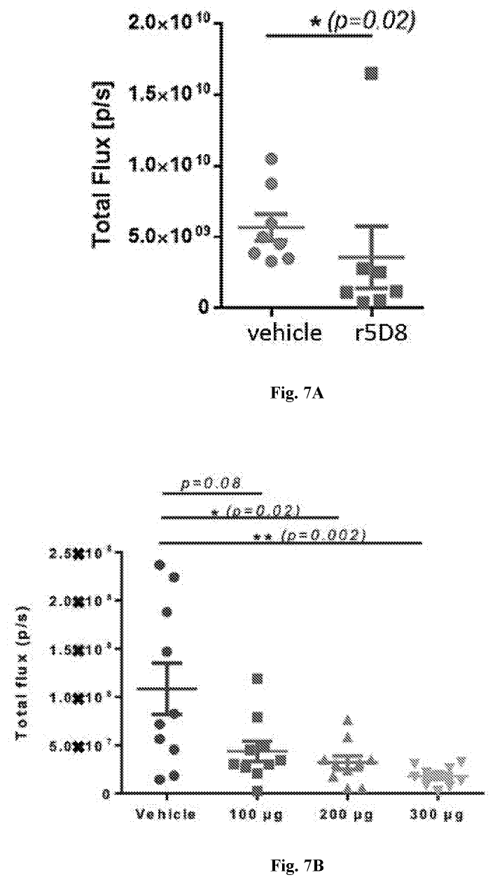

In an orthotopic GBM tumor model using a luciferase expressing human cell line U251, r5D8 significantly reduced tumor volumes in mice administered 300 μg r5D8 and h5D8 by intraperitoneal (IP) injection twice a week. Results of this study are shown in FIG. 7A (quantitation at day 26 post treatment). This experiment was also conducted using humanized h5D8 mice treated with 200 μg or 300 μg showed a statistically significant reduction in tumor after 7 days of treatment. FIG. 7B shows data from mice inoculated with luciferase expressing human U251 GBM cells and then treated with 100, 200 or 300 μg of h5D8 or vehicle twice a week. Tumor size was determined by bioluminescence (Xenogen IVIS Spectrum) on day 7. The graph shows individual tumor measurements with horizontal bars indicating mean±SEM. Statistical significance was calculated using the unpaired non-parametric Mann-Whitney U-test.

### Methods

U251 cells stably expressing luciferase were harvested, washed in PBS, centrifuged at 400 g for 5 min, resuspended in PBS and counted with an automated cell counter (Countess, Invitrogen). Cells were kept on ice to maintain optimal viability. Mice were anaesthetized with intraperitoneal administration of Ketamine (Ketolar50®)/Xylacine (Rompún®) (75 mg/kg and 10 mg/kg respectively). Each mouse was carefully placed in the stereotactic device and immobilized. Hair from the head was removed with depilatory cream, and the head skin was cut with a scalpel to expose the skull. A small incision was carefully made with a drill in the coordinates 1.8 mm lateral and 1 mm anterior to the Lambda. 5 μL of cells were inoculated using a Hamilton 30G syringe into the right corpus striatum, at 2.5 mm of depth. Head incision was closed with Hystoacryl tissue adhesive (Braun) and mice were injected with subcutaneous analgesic Meloxicam (Metacam®) (1 mg/kg). The final cell number implanted into each mouse was 3×10⁵.

Mice were treated twice a week with h5D8 administered intraperitoneally. Treatment was initiated on day 0, immediately after tumor cell inoculation. Mice received a total of 2 doses of h5D8 or vehicle control.

Body weight and tumor volume: Body weight was measured 2 times/week and tumor growth was quantified by bioluminescence on day 7 (Xenogen IVIS Spectrum). To quantify bioluminescence activity in vivo, mice were anaesthetized using isofluorane, and injected intraperitoneally with luciferin substrate (PerkinElmer) (167 μg/kg).

Tumor size as determined by bioluminescence (Xenogen IVIS Spectrum) was evaluated at day 7. The individual tumor measurements and mean±SEM for each treatment group were calculated. Statistical significance was determined by the unpaired non-parametric Mann-Whitney U-test.

### Example 12

### h5D8 Inhibits Tumor Growth in a Mouse Model of Ovarian Cancer

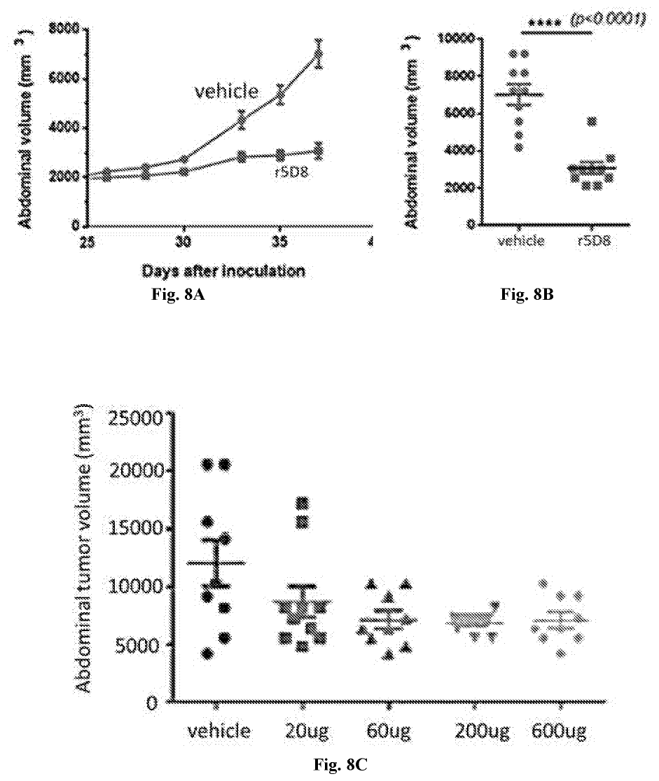

The efficacy of r5D8 was evaluated in two other syngeneic tumor models. In the ovarian orthotopic tumor model ID8, IP administration of 300 μg r5D8 twice weekly significantly inhibited tumor growth as measured by abdominal volume (FIGS. 8A and 8B). Results in FIG. 8C show that h5D8 also reduced tumor volume at a dose of 200 μg and above.

### Methods

ID8 cells were cultured in Dulbecco's Modified Eagle Medium (DMEM) (Gibco, Invitrogen), supplemented with 10% Fetal Bovine Serum (FBS) (Gibco, Invitrogen), 40 U/mL Penicillin and 40 μg/mL Streptomycin (PenStrep) (Gibco, Invitrogen) and 0.25 μg/mL Plasmocin (Invivogen).

The ID8 cells were harvested, washed in PBS, centrifuged at 400 g for 5 min and resuspended in PBS. Cells were kept on ice to maintain optimal viability and 200 μL of the cell suspension was injected intraperitoneally with a 27G needle. The final cell number implanted into mice was 5×10⁶.

Mice were treated twice weekly with h5D8 administered ip at different doses as indicated. Body weights were measured 2 times/week and tumor progression was monitored by measuring abdominal girth using a caliper (Fisher Scientific).

### Example 13

### r5D8 Inhibits Tumor Growth in a Mouse Model of Colorectal Cancer

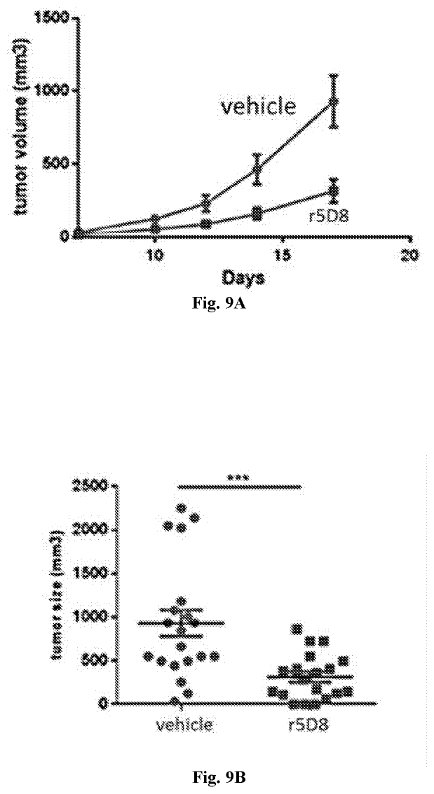

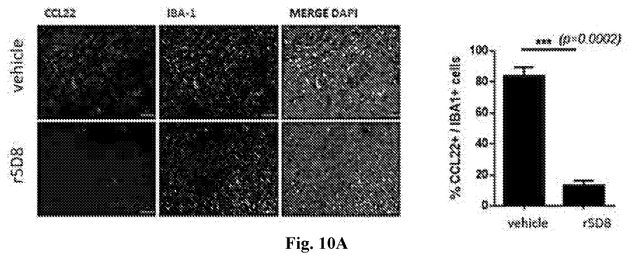

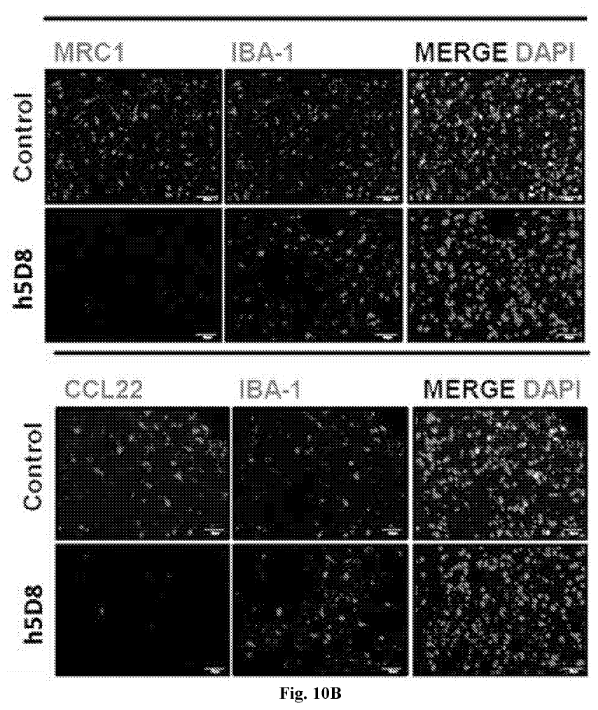

In mice with subcutaneous colon CT26 tumors, r5D8 (administered 300 μg IP twice weekly) significantly inhibited tumor growth (FIGS. 9A and 9B).

### Methods

CT26 cells were cultured in Roswell Park Memorial Institute medium (RPMI [Gibco, Invitrogen]), supplemented with 10% Fetal Bovine Serum (FBS), 40 U/mL penicillin and 40 μg/mL streptomycin (PenStrep) and 0.25 μg/mL Plasmocin.

CT26 cells (8×10⁵)were trypsinized, rinsed with PBS, centrifuged at 400 g for 5 minutes and resuspended in 100 μL PBS. Cells were kept on ice to avoid cell death. The CT26 cells were administered to mice via subcutaneous injection using a 27G needle.

300 μg r5D8, or vehicle control, was administered to the mice via intraperitoneal injection (IP) twice weekly from day 3 post CT26 cell implant.

Body weight and tumor volumes were measured three times per week. Tumor volume was measured using a caliper (Fisher Scientific).

### Example 14

### r5D8 Reduces Inflammatory Infiltration in Tumor Models

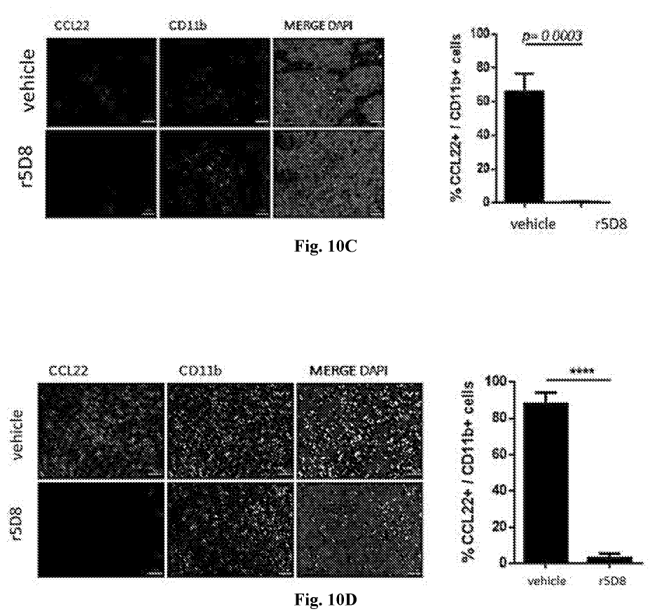

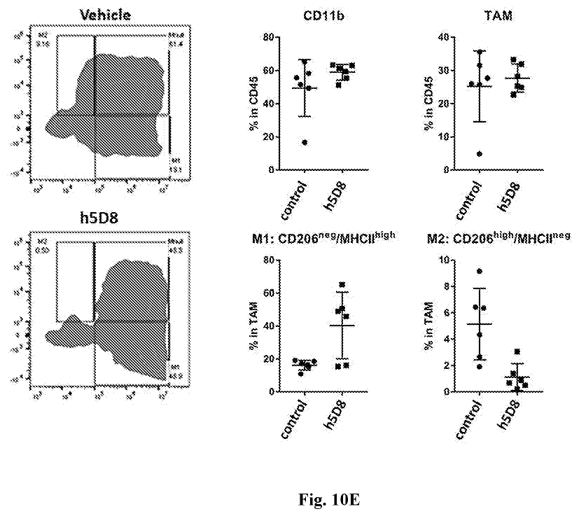

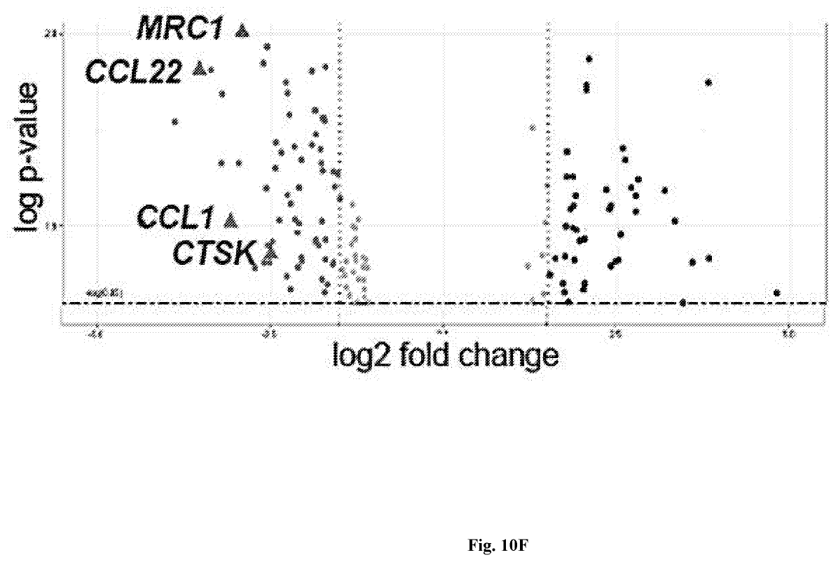

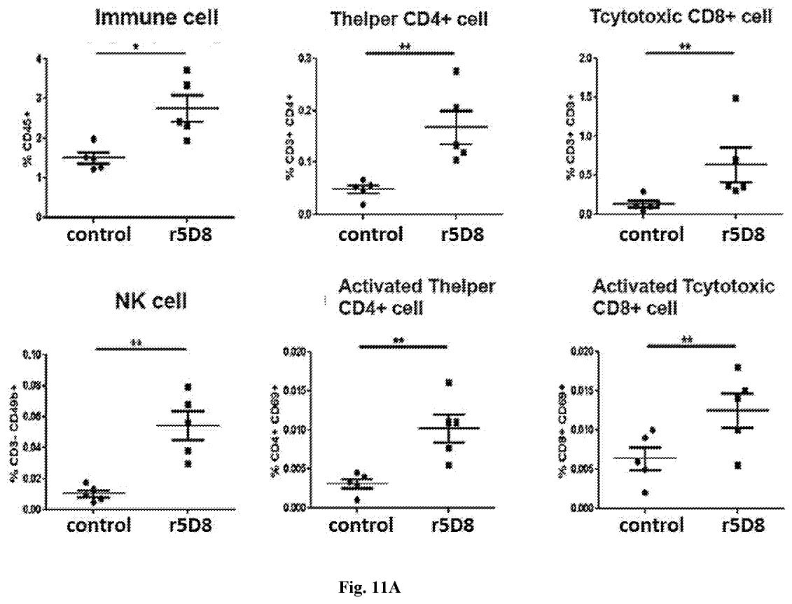

In the U251 GBM orthotopic model, expression of CCL22, a marker of M2 polarized macrophages, was significantly decreased in tumors treated with r5D8 as shown in FIG. 10A. This finding was also confirmed in a physiologically relevant organotypic tissue slice culture model using h5D8 in which three patient samples showed a significant decrease in CCL22 and CD206 (MRC1) expression (also a marker of M2 macrophages) after treatment with as shown in FIG. 10B (compare upper left, control, to lower right, treated, for both MRC1 and CCL22). Furthermore, r5D8 also decreased CCL22⁺M2 macrophages in syngeneic ID8 (FIG. 10C) and CT26 (FIG. 10D) tumors in immunocompetent mice. H5D8 treatment also programmed macrophages towards an immune-stimulatory phenotype in the syngeneic CT26 tumor model (FIG. 10E). h5D8 treatment increased macrophages with an M1 phenotype as indicated by an increased CD206 negative/MHCII positive fraction, and decreased macrophages with an M2 phenotype as indicated by a decreased CD206 positive/MHCII negative fraction. FIG. 10F shows gene expression data from monocytes cultured in the conditioned media of U251 cells with LIF knock down. MRC1, CCL2, CCL1 and CTSK (denoted with triangles) all showed significant reductions in expression.

### Example 15

### r5D8 Increases Non-Myeloid Effector Cells

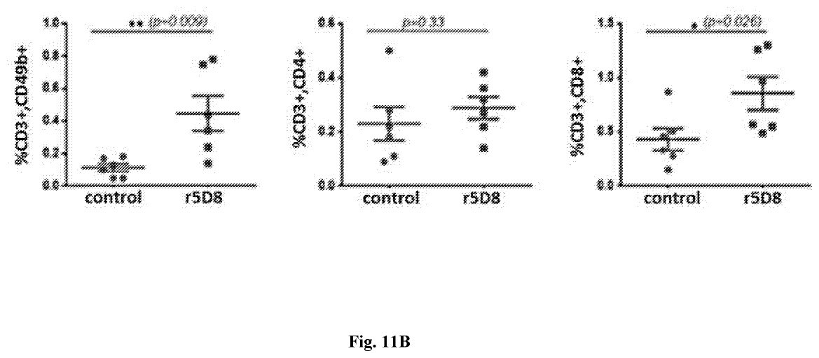

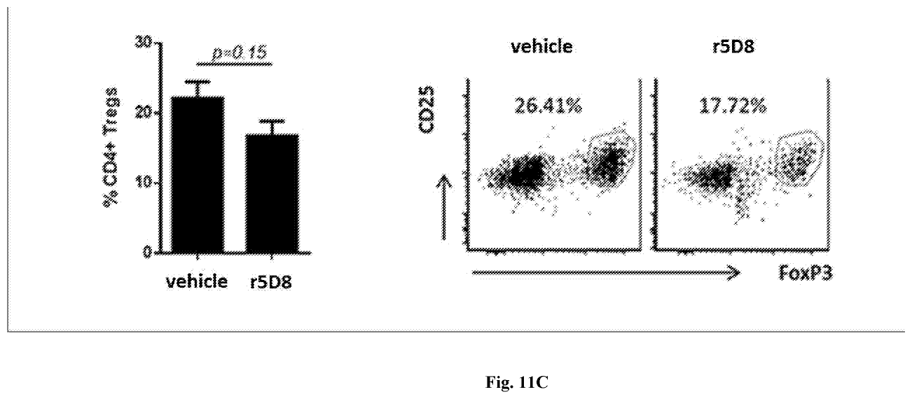

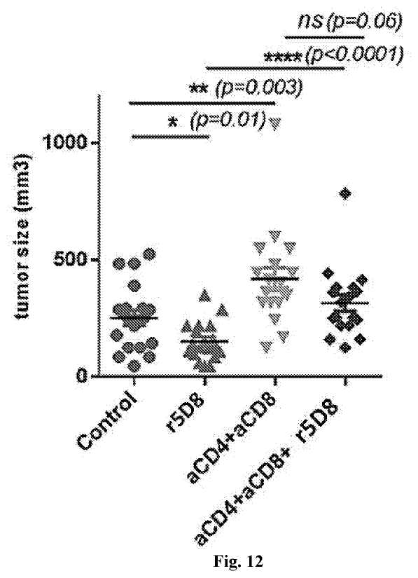

To investigate additional immune mechanisms, the effect of r5D8 on T cells and other non-myeloid immune effector cells within the tumor microenvironment were evaluated. In the ovarian orthotopic ID8 syngeneic model, r5D8 treatment resulted in an increase in intratumoral NK cells and an increase in total and activated CD4⁺and CD8⁺T cells as shown in FIG. 11A. Similarly, in the colon syngeneic CT26 tumor model, r5D8 increased intratumoral NK cells, increased CD4+and CD8+T cells and trended to decrease CD4⁺CD25⁺FoxP3⁺T-reg cells as shown in FIG. 11B. A trend for a decrease in CD4⁺CD25⁺FoxP3⁺T-reg cells was also observed in the syngeneic orthotopic KLN205 tumor model following r5D8treatment as shown in FIG. 11C. Consistent with a requirement for T cells to mediate efficacy, depletion of CD4⁺and CD8⁺T cells in the CT26 model inhibited the anti-tumor efficacy of r5D8 as shown in FIG. 12.

### Methods for T Cell Depletion

CT26 cells were cultured in RPMI culture medium (Gibco, Invitrogen), supplemented with 10% Fetal Bovine Serum (FBS [Gibco, Invitrogen]), 40 U/mL penicillin and 40 μg/mL streptomycin (PenStrep [Gibco, Invitrogen]) and 0.25 μg/mL Plasmocin (Invivogen). CT26 cells (5×10⁵) were collected, rinsed with PBS, centrifuged at 400 g for 5 minutes and resuspended in 100 μL PBS. Cells were kept on ice to avoid cell death. The CT26 cells were administered in both flanks to mice via subcutaneous injection using a 27G syringe. Mice were treated twice weekly with r5D8 administered intraperitoneally as indicated in the study design. Vehicle control (PBS), rat r5D8, and/or anti-CD4 and anti-CD8 was administered to the mice via intraperitoneal injection (IP) twice weekly as stated in the study design. All antibody treatments were administered concomitantly.

### Example 16

### Crystal Structure of h5D8 in Complex with Human LIF

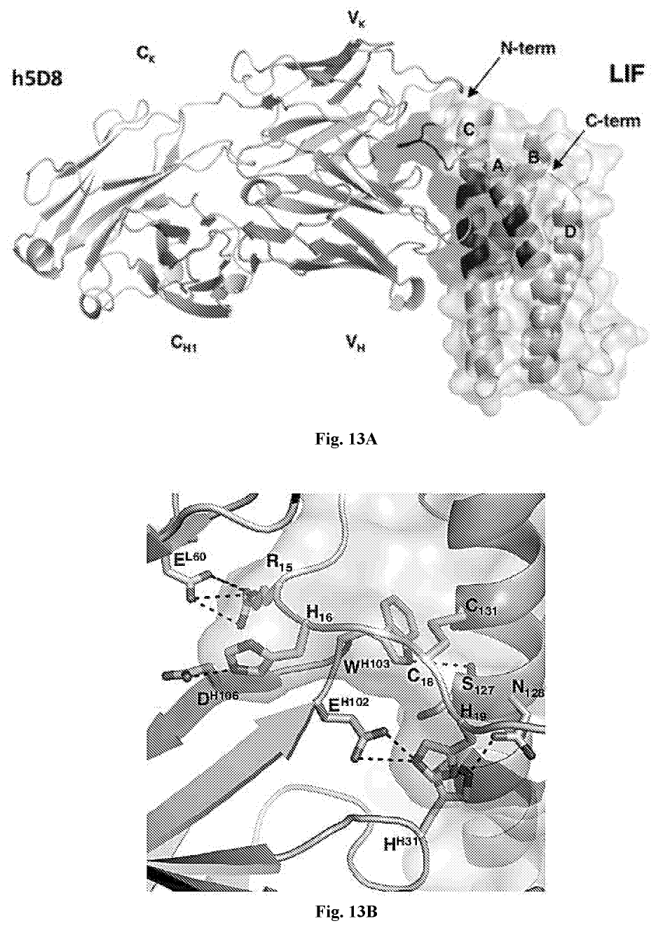

The crystal structure of h5D8 was solved to a resolution of 3.1 angstroms in order to determine the epitope on LIF that h5D8 was bound to and to determine residues of h5D8 that participate in binding. The co-crystal structure revealed that the N-terminal loop of LIF is centrally positioned between the light and heavy chain variable regions of h5D8 (FIG. 13A). In addition, h5D8 interacts with residues on helix A and C of LIF, thereby forming a discontinuous and conformational epitope. Binding is driven by several salt-bridges, H-bonds and Van der Waals interactions (Table 7, FIG. 13B). The h5D8 epitope of LIF spans the region of interaction with gp130. See Boulanger, M. J., Bankovich, A. J., Kortemme, T., Baker, D. & Garcia, K. C. Convergent mechanisms for recognition of divergent cytokines by the shared signaling receptor gp130. Molecular cell 12, 577-589 (2003). The results are summarized below in Table 7 and depicted in FIG. 13.

TABLE 7
Summary of X-Ray crystal structure for h5D8 in complex with human LIF

| LIF Residue | Interaction | |
|---|---|---|
| (epitope) | type | h5D8 Residue (paratope, Kabat numbering) |
| Ala13 | VDW | L-Tyr49, L-Asn53 |
| Ile14-O | HB | L-Ser50-OG |
| Ile | VDW | L-His30, L-Tyr32, L-Tyr49, L-Ser50 |
| | | H-Trp97 |
| Arg15-NE | SB | L-Glu55-OE1, L-Glu55-OE2 |
| Arg15-NH1 | SB | L-Glu55-OE1, L-Glu55-OE2 |
| Arg15-NH2 | SB | L-Glu55-OE1, L-Glu55-OE2 |
| Arg15-O | HB | L-Asn34-ND2 |
| Arg15 | VDW | L-Asn34, L-Leu46, L-Tyr49, L-Glu55, L-Ser56 |
| | | H-Glu96, H-Trp97, H-Asp98, H-Leu99, H-Asp101 |
| His16-NE2 | SB | H-Asp101-OD2 |
| His16 | VDW | L-Tyr32, L-Asn34, L-Met89 |
| | | H-Trp95, H-Glu96, H-Trp97, H-Asp101 |
| Pro17 | VDW | L-Tyr32, L-Ala91 |
| | | H-Trp97 |
| Cys18 | VDW | L-Tyr32 |
| | | H-Trp33, H-Trp97 |
| His19-NE2 | SB | H-Glu96-OE1, H-Glu96-OE2 |
| His19 | VDW | H-His31, H-Trp33, H-Glu96 |
| Asn20-OD1 | HB | H-Lys52-NZ |
| Asn20-ND2 | HB | H-Asp53-OD1 |
| Asn20 | VDW | H-Trp33, H-Lys52, H-Asp53 |
| Gln25-NE2 | HB | H-Asp53-OD2 |
| Gln25 | VDW | H-His31, H-Ser52C, H-Asp53 |
| Gln29 | VDW | H-His31 |
| Gln32 | VDW | H-Lys52B |
| Asp120-OD2 | HB | H-Ser30-OG |
| Asp120 | VDW | H-Thr28, H-Ser30 |
| Arg123-NE | HB | H-Thr28-OG |
| Arg123 | VDW | H-Thr28 |
| Gly124 | VDW | H-His31 |
| Leu125 | VDW | H-His31 |
| Ser127-OG | HB | H-Asp98-OD2 |
| Ser127-O | HB | H-Trp97-NE1 |
| Ser127 | VDW | H-His31, H-Trp97, H-Asp98 |
| Asn128-OD1 | HB | H-His31-NE2 |
| Asn128 | VDW | H-His31 |
| Leu130 | VDW | H-Trp97 |
| Cys131 | VDW | H-Trp97 |
| Cys134 | VDW | H-Trp97 |
| Ser135-O | HB | L-His30-NE2 |
| Ser135 | VDW | L-His30 |
| His138 | VDW | L-His30 |

VDW, Van der Waals low energy binding; HB, hydrogen bond (medium energy binding); SB, salt bridge (high energy binding)

### Methods

LIF was transiently expressed in HEK 293S (Gnt I^{-/-}) cells and purified using Ni-NTA affinity chromatography, followed by gel-filtration chromatography in 20 mM Tris pH 8.0 and 150 mM NaCl. The recombinant h5D8 Fab was transiently expressed in HEK 293F cells and purified using KappaSelect affinity chromatography, followed by cation exchange chromatography. Purified h5D8 Fab and LIF were mixed at a 1:2.5 molar ratio and incubated at room temperature for 30 min prior to deglycosylation using EndoH. Gel-filtration chromatography was subsequently used to purify the complex. The complex was concentrated to 20 mg/mL and set up for crystallization trials using sparse matrix screens. Crystals formed at 4° C. in a condition containing 19% (v/v) isopropanol, 19% (w/v) PEG 4000, 5% (v/v) glycerol, 0.095 M sodium citrate pH 5.6. The crystal diffracted to a resolution of 3.1 Å at the 08ID-1 beamline at the Canadian Light Source (CLS). Data were collected, processed and scaled using XDS as per Kabsch et al. Xds. Acta crystallographica. Section D, Biological crystallography 66, 125-132 (2010). Structures were determined by molecular replacement using Phaser as per McCoy et al. Phaser crystallographic software. J Appl Crystallogr 40, 658-674 (2007). Several iterations of model building and refinement were performed using Coot and phenix.refine until the structures converged to an acceptable R_{work} and R_{free}. See Emsley et al. Features and development of Coot. Acta crystallographica. Section D, Biological crystallography 66, 486-501 (2010); and Adams, et al. PHENIX: a comprehensive Python-based system for macromolecular structure solution. Acta crystallographica. Section D, Biological crystallography 66, 213-221 (2010) respectively. The figures were generated in PyMOL (The PyMOL Molecular Graphics System, Version 2.0 Schrödinger, LLC).

### Example 17

### h5D8 has High Specificity for LIF

Binding of h5D8 to other LIF family members was tested to determine the binding specificity. Using Octet96 analysis h5D8 binding to human LIF is approximately 100-fold greater than binding to LIFs highest homology IL-6 family member Oncostatin M (OSM) when both proteins are produced in E. coli. When both proteins are produced in a mammalian system h5D8 exhibits no binding to OSM. Data are summarized in Table 8.

TABLE 8
Summary of h5D8 Affinity Measurements for Cytokines as
Measured by Octet

| | KD [M] | kon [1/Ms] | kdis [1/s] |
|---|---|---|---|
| h5D8 + huLIF | 4.3E−10+/−2.0E−11 | 3.1E+05+/−3.1E+03 | 1.3E−04+/ |
| (E. coli) | | | −5.8E−06 |
| h5D8 + huLIF | 1.3E−09+/−7.2E−11 | 1.2E+05+/−1.3E+03 | 1.5E−04+/ |
| (mammalian) | | | −8.5E−06 |
| h5D8 + huOSM | 3.6E−08+/−1.4E−09 | 8.5E+04+/−3.1E+03 | 3.1E−03+/ |
| (E. coli) | | | −4.1E−05 |
| h5D8 + huOSM | ND | ND | ND |
| (mammalian) | | | |
| h5D8 + huIL−6 | ND | ND | ND |
| (E. coli) | | | |

ND = no binding

### Methods

Octet Binding Experiments: Reagents were used and prepared as per manufacturer's provided manual. A Basic Kinetics Experiment was performed using Octet Data Acquisition software ver. 9.0.0.26 as follows: Setup of sensors/program: i) Equilibration (60 seconds); ii) Loading (15 seconds); iii) Baseline (60 seconds); iv) Association (180 seconds); and v) Dissociation (600 seconds)

Octet Affinity of h5D8 for cytokines: A Basic Kinetics Experiment was performed using Octet Data Acquisition software ver. 9.0.0.26 as follows: Amine Reactive 2ndGeneration Biosensors (AR2G) were hydrated for a minimum of 15 minutes in water. Amine conjugation of h5D8 to the biosensors was performed according to ForteBio Technical Note 26 (please see References) using the Amine Coupling Second Generation Kit. Dip steps were as performed at 30° C., 1000 rpm as follows: i) 60 seconds Equilibration in water; ii) 300 seconds Activation in 20 mM ECD, 10 mM sulfo-NHS in water; iii) 600 second Immobilization of 10 μg/ml h5D8 in 10 mM Sodium Acetate, pH 6.0; iv) 300 seconds Quench in 1M Ethanolamine, pH 8.5; v) 120 seconds Baseline in water. Kinetics experiments were then performed with the following Dip and Read steps at 30° C., 1000rpm: vi) 60 seconds Baseline in 1× kinetics buffer; vii) 180 seconds Association of appropriate serial dilutions of a cytokine in 1× kinetics buffer; viii) 300 seconds Dissociation in 1× kinetics buffer; ix) Three Regeneration/Neutralization cycles alternating between 10 mM glycine pH 2.0 and 1× kinetics buffer respectively (5 seconds in each for 3 cycles). Following regeneration, the biosensors were reused for subsequent binding analyses.

Human recombinant LIF produced from mammalian cells was from ACROBiosystems (LIF-H521b); human recombinant OSM produced in mammalian cells was from R & D (8475-OM/CF); and human recombinant OSM produced in E. coli cells was from R & D (295-OM-050/CF).

### Example 18

### Crystal Structure of h5D8 Fab

Five crystal structures of the h5D8 Fab under a wide spectrum of chemical conditions were determined. The high resolutions of these structures indicate that the conformations of CDR residues are associated with minor flexibility, and are highly similar in different chemical environments. A unique feature of this antibody is the presence of a non-canonical cysteine in position 100 of the variable heavy region. Structure analysis shows that the cysteine is unpaired and largely inaccessible to the solvent.

H5D8 Fab was obtained by papain digestion of its IgG, followed by purification using standard affinity, ion exchange and size chromatography techniques. Crystals were obtained using vapor diffusion methods and allowed to determine five crystal structures ranging between 1.65 Å to 2.0 Å in resolution. All structures were solved in the same crystallographic space group and with similar unit cell dimensions (P212121, a˜53.8 Å, b˜66.5 Å, c˜143.3 Å), despite crystallization conditions ranging across five different pH levels: 5.6, 6.0, 6.5, 7.5 and 8.5. As such, these crystal structures allow for comparison of the three-dimensional disposition of h5D8 Fab unimpeded by crystal packing artefacts and across a wide spectrum of chemical conditions.

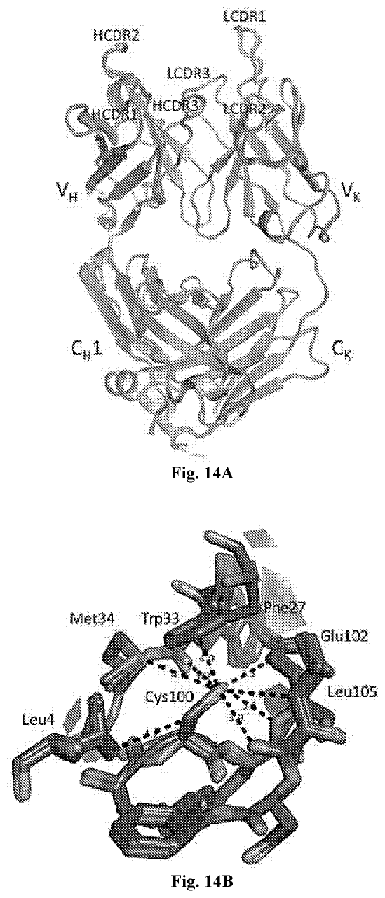

Electron density was observed for all complementarity determining region (CDR) residues, which were subsequently modeled. Noticeably, LCDR1 and HCDR2 adopted elongated conformations that together with shallow LCDR3 and HCDR3 regions formed a binding groove at the center of the paratope (FIG. 14A). The five structures were highly similar across all residues, with all-atoms root mean square deviations ranging between 0.197 Å and 0.327 Å (FIG. 14A). These results indicated that the conformations of CDR residues were maintained in various chemical environments, including pH levels ranging between 5.6 and 8.5 and ionic strengths ranging between 150 mM and 1 M. Analysis of the electrostatic surface of the h5D8 paratope revealed that positively and negatively charged regions equally contributed to hydrophilic properties, with no prevalent hydrophobic patches. h5D8 has the uncommon feature of a non-canonical cysteine at the base of HCDR3 (Cys100). In all five structures, this free cysteine is ordered and does not form any disulfide scrambles. Additionally, it is not modified by the addition of Cys (cysteinylation) or glutathione (glutathiolation) and makes Van der Waals interactions (3.5-4.3 Å distances) with main chain and side chain atoms of Leu4, Phe27, Trp33, Met34, Glu102 and Leu105 of the heavy chain (FIG. 14B). Finally, Cys100 is a predominantly buried structural residue that appears to be involved in mediating the conformations of CDR1 and HCDR3. It is thus unlikely to have reactivity with other cysteines, as observed by a homogeneous disposition of this region in our five crystal structures.

### Methods

H5D8-1 IgG was obtained from Catalent Biologics and was formulated in 25 mM histidine, 6% sucrose, 0.01% polysorbate 80, at pH 6.0. The formulated IgG was extensively buffer-exchanged into PBS using a 10K MWCO concentrator (Millipore) prior to digestion with 1:100 microgram papain (Sigma) for 1 hour at 37° C. in PBS, 1.25 mM EDTA, 10 mM cysteine. The papain-digested IgG was flown through a Protein A column (GE Healthcare) using an AKTA Start chromatography system (GE Healthcare). The Protein A flow-through, which contained the h5D8 Fab was recovered and buffer-exchanged into 20 mM sodium acetate, pH 5.6 using a 10K MWCO concentrator (Millipore). The resulting sample was loaded onto a Mono S cation exchange column (GE Healthcare) using an AKTA Pure chromatography system (GE Healthcare). Elution with a gradient of 1 M potassium chloride resulted in a predominant h5D8 Fab peak that was recovered, concentrated and purified to size homogeneity using a Superdex 200 Increase gel filtration column (GE Healthcare) in 20 mM Tris-HCl, 150 mM sodium chloride, at pH 8.0. The high purity of the h5D8 Fab was confirmed by SDS-PAGE under reducing and non-reducing conditions.

Purified h5D8 Fab was concentrated to 25 mg/mL using a 10K MWCO concentrator (Millipore). An Oryx 4 dispenser (Douglas Instruments) was used to set up vapor diffusion crystallization experiments with sparse matrix 96-conditions commercial screens JCSG TOP96 (Rigaku Reagents) and MCSG-1 (Anatrace) at 20° C. Crystals were obtained and harvested after four days in the following five crystallization conditions: 1) 0.085 M sodium citrate, 25.5% (w/v) PEG 4000, 0.17 M ammonium acetate, 15% (v/v) glycerol, pH 5.6; 2) 0.1 M MES, 20% (w/v) PEG 6000, 1 M lithium chloride, pH 6.0; 3) 0.1 M IVIES, 20% (w/v) PEG 4000, 0.6 M sodium chloride, pH 6.5; 4) 0.085 M sodium HEPES, 17% (w/v) PEG 4000, 8.5% (v/v) 2-propanol, 15% (v/v) glycerol, pH 7.5; and 5) 0.08 M Tris, 24% (w/v) PEG 4000, 0.16 M magnesium chloride, 20% (v/v) glycerol, pH 8.5. Prior to flash-freezing in liquid nitrogen, mother liquors containing the crystals were supplemented with 5-15% (v/v) glycerol or 10% (v/v) ethylene glycol, as required. Crystals were subjected to X-ray synchrotron radiation at the Advanced Photon Source, beamline 23-ID-D (Chicago, Ill.) and diffraction patterns were recorded on a Pilatus3 6M detector. Data were processed using XDS and structures were determined by molecular replacement using Phaser. Refinement was carried out in PHENIX with iterative model building in Coot. Figures were generated in PyMOL. All software were accessed through SBGrid.

### Example 19

### Mutations at Cysteine 100 of h5D8 Preserve Binding

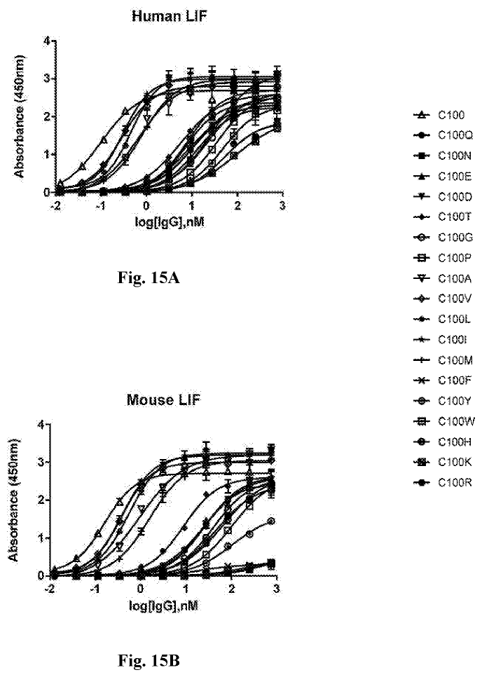

Analysis of h5D8 revealed a free cysteine residue at position 100 (C100) in the variable region of the heavy chain. H5D8 variants were generated by substituting C100 with each naturally occurring amino acid in order to characterize binding to and affinity for human and mouse LIF. Binding was characterized using ELISA and Octet assay. Results are summarized in Table 9. ELISA EC50 curves are shown in FIG. 15 (FIG. 15A human LIF and FIG. 15B Mouse LIF).

TABLE 9
Summary of affinities determined by Octet assay and EC50 determined by ELISA

| | Affinity/k_{D} (M) | | Binding EC50 (nM) | |
|---|---|---|---|---|
| Mutation | human LIF | mouse LIF | human LIF | mouse LIF |
| C100 | <1.0E−12 ± 2.25E−11 | 9.946E−11 ± 8.272E−12 | 0.09878 | 0.1605 |
| C100S | 8.311E−10 ± 5.886E−11 | 2.793E−09 ± 5.925E−11 | n.d. | n.d. |
| C100Q | 3.87E−09 ± 1.55E−10 | 2.84E−09 ± 4.85E−11 | 10.18 | 26.33 |
| C100N | 5.59E−09 ± 1.01E−10 | 6.68E−09 ± 9.8E−11 | 13.18 | 45.87 |
| C100E | 2.67E−09 ± 4.64E−11 | 4.1E−09 ± 7.56E−11 | 7.179 | 25.3 |
| C100D | 2.02E−09 ± 8.08E−11 | 6.49E−09 ± 7.16E−11 | 11.89 | 22.88 |
| C100T | 4.36E−10 ± 2.1E−11 | 1.02E−09 ± 1.77E−11 | 5.575 | 8.753 |
| C100G | 2.49E−09 ± 4.2E−11 | 3.33E−09 ± 5.42E−11 | 21.94 | 40.17 |
| C100P | 2.74E−10 ± 2.97E−10 | <1.0E−12 ± 7.64E−10 | 34.44 | 101.9 |
| C100A | <1.0E−12 ± 2.713E−11 | <1.0E−12 ± 1.512E−11 | 0.6705 | 0.9532 |
| C100V | <1.0E−12 ± 1.805E−11 | <1.0E−12 ± 8.086E−12 | 0.2785 | 0.3647 |
| C100L | <1.0E−12 ± 1.963E−11 | 1.998E−10 ± 1.055E−11 | 0.454 | 0.547 |
| C100I | <1.0E−12 ± 1.424E−11 | 3.361E−11 ± 7.545E−12 | 0.299 | 0.3916 |
| C100M | 1.155E−09 ± 3.400E−11 | 2.676E−09 ± 2.449E−11 | 0.7852 | 1.563 |
| C100F | 4.376E−09 ± 1.127E−10 | 1.147E−08 ± 9.099E−11 | 8.932 | 21.53 |
| C100Y | 1.444E−08 ± 1.159E−09 | 2.514E−08 ± 2.047E−09 | n.d. | n.d. |
| C100W | 2.508E−08 ± 7.036E−09 | 4.819E−08 ± 4.388E−09 | n.d. | n.d. |
| C100H | 1.304E−10 ± 1.416E−10 | 4.284E−09 ± 1.231E−10 | 8.254 | n.d. |
| C100K | 7.477E−08 ± 1.581E−09 | 6.053E−08 ± 2.589E−09 | n.d. | n.d. |
| C100R | 1.455E−07 ± 6.964E−09 | 5.142E−08 ± 3.247E−09 | n.d. | n.d. |

### Methods

ELISA: Binding of h5D8 C100 variants to human and mouse LIF was determined by ELISA. Recombinant human or mouse LIF protein was coated on Maxisorp 384-well plates at 1 ug/mL overnight at 4° C. Plates were blocked with 1× blocking buffer for 2 hours at room temperature. Titrations of each h5D8 C100 variants were added and allowed to bind for 1 hour at room temperature. Plates were washed three times with PBS+0.05% Tween-20. HRP-conjugated anti-human IgG was added and allowed to bind for 30 min at room temperature. Plates were washed three times with PBS+0.05% Tween-20 and developed using 1× TMB substrate. The reaction was stopped with 1M HCl and absorbance at 450 nm was measured. Generation of figures and non-linear regression analysis was performed using Graphpad Prism.

Octet RED96: The affinity of h5D8 C100 variants to human and mouse LIF was determined by BLI using the Octet RED96 system. h5D8 C100 variants were loaded onto Anti-Human Fc biosensors at 7.5 μg/mL following a 30 second baseline in 1× kinetics buffer. Titrations of human or mouse LIF protein were associated to the loaded biosensors for 90 seconds and allowed to dissociate in 1× kinetics buffer for 300 seconds. KDs were calculated by the data analysis software using a 1:1 global fit model.

### Example 20

### h5D8 Blocks Binding of LIF to gp130 In Vitro

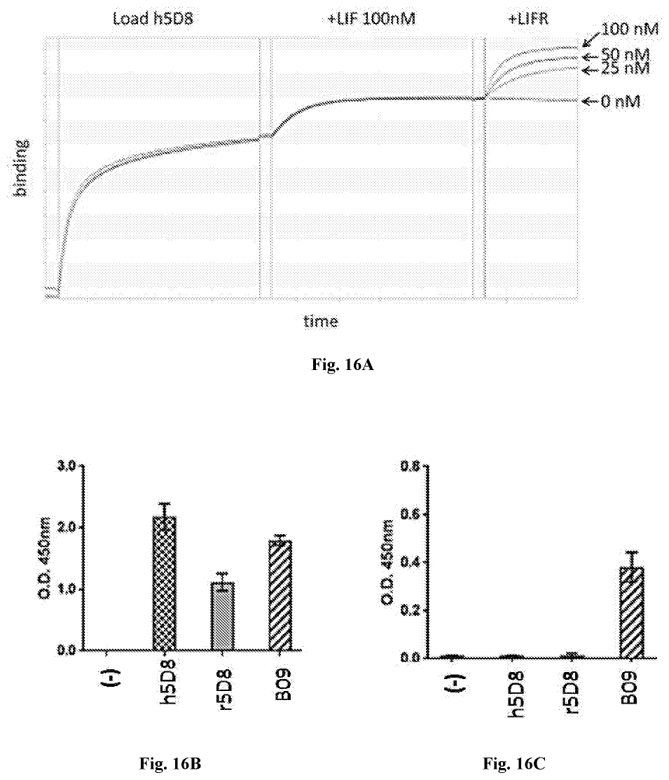

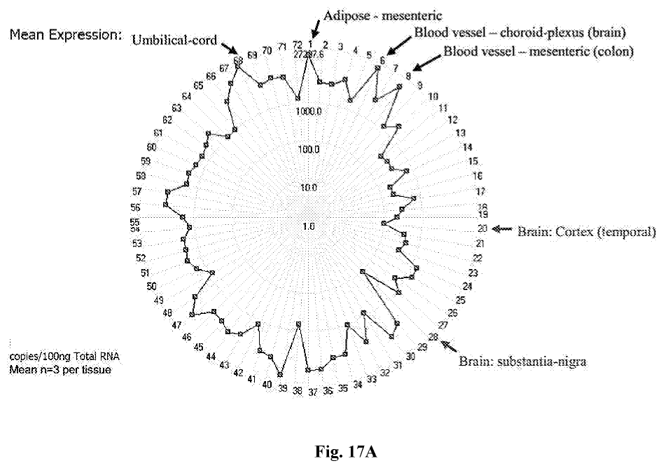

To determine whether h5D8 prevented LIF from binding to LIFR, a molecular binding assay using the Octet RED 96 platform was performed. H5D8 was loaded onto AHC biosensors by anti-human Fc capture. Then, the biosensors were dipped in LIF and, as expected, association was observed (FIG. 16A, middle third). Subsequently, the biosensors were dipped in different concentrations of LIFR. A dose-dependent association was observed (FIG. 16A, right third). The control experiment demonstrated that this association was LIF-specific (not shown), and not due to a non-specific interaction of LIFR with h5D8 or with the biosensors.

To further characterize the binding of h5D8 and LIF, a series of ELISA binding experiments was conducted. H5D8 and LIF were pre-incubated and were then introduced to plates coated with either recombinant human LIFR (hLIFR) or gp130. The lack of binding between the h5D8/LIF complex and the coated substrate would indicate that h5D8 in some way disrupted the binding of LIF to the receptor. Additionally, control antibodies that either did not bind LIF (isotype control, indicated by (−)) or that bind LIF at known binding sites (B09 does not compete with either gp130 or LIFR for LIF binding; r5D8 is the rat parental version of h5D8) were also used. The ELISA results demonstrated that the h5D8/LIF complex was able to bind hLIFR (as was r5D8/LIF complex), indicating that these antibodies did not prevent the LIF/LIFR association (FIG. 16A). In contrast, the h5D8/LIF complex (and a r5D8/LIF complex) was not able to bind recombinant human gp130 (FIG. 16B). This indicates that the gp130 binding site of LIF was affected when LIF was bound to h5D8.

### Example 21

### LIF and LIFR Expression in Human Tissues

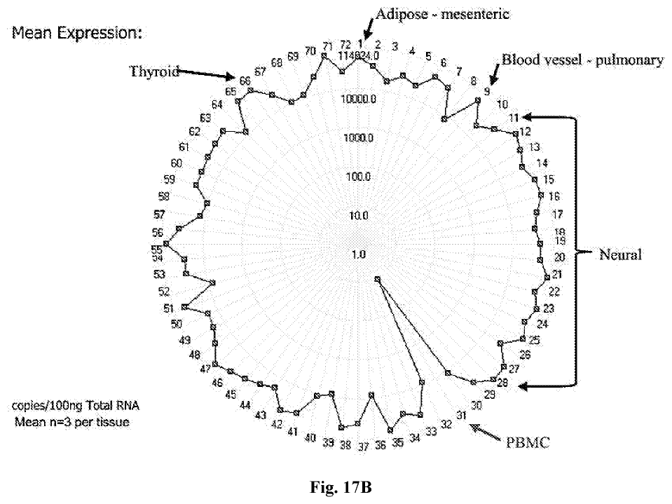

Quantitative real-time PCR was performed on many different types of human tissue in order to determine expression levels of LIF and LIFR. The mean expression levels shown in FIGS. 17A and 17B are given as copies per 100 ng of total RNA. Most tissues expressed at least 100 copies per 100 ng of total RNA. LIF mRNA expression was highest in human adipose tissue (mesenteric-ileum [1]), blood-vessel tissue (choroid-plexus [6] and mesenteric [8]) and umbilical cord [68] tissue and lowest in brain tissue (cortex [20] and substantia-nigra [28]). LIFR mRNA expression was highest in human adipose tissue (mesenteric-ileum [1]), blood vessel tissue (pulmonary [9]), brain tissue [11-28] and thyroid [66] tissue and was lowest in PBMCs [31]. LIF and LIFR mRNA expression levels in cynomolgus tissues were similar to those observed in human tissues, wherein LIF expression was high in adipose tissue and LIFR expression was high in adipose tissue and low in PBMCs (data not shown).

The tissue numbering for FIG. 17A and FIG. 17B is: 1—adipose (mesenteric-ileum); 2—adrenal gland; 3—bladder; 4—bladder (trigone); 5—blood-vessel (cerebral: middle-cerebral-artery); 6—blood vessel (choroid-plexus); 7—blood vessel (coronary artery); 8—blood vessel (mesenteric (colon)); 9—blood vessel (pulmonary); 10—blood vessel (renal); 11—brain (amygdala); 12—brain (caudate); 13—brain (cerebellum); 14 brain—(cortex: cingulate-anterior); 15—brain (cortex: cingulate-posterior); 16—brain (cortex: frontal-lateral); 17—brain (cortex: frontal-medial); 18—brain (cortex: occipital); 19—brain (cortex: parietal); 20—brain (cortex: temporal); 21—brain (dorsal-raphe-nucleus); 22—brain (hippocampus); 23—brain (hypothalamus: anterior); 24—brain (hypothalamus: posterior); 25—brain (locus coeruleus); 26—brain (medulla oblongata); 27—brain (nucleus accumbens); 28—brain (substantia nigra); 29—breast; 30—caecum; 31—peripheral blood mononuclear cell (PBMCs); 32—colon; 33—dorsal root ganlia (DRG); 34—duodenum; 35—fallopian tube; 36—gallbladder; 37—heart (left atrium); 38—heart (left ventricle); 39—ileum; 40—jejunum; 41—kidney (cortex); 42—kidney (medulla); 43—kidney (pelvis); 44—liver (parenchyma); 45—liver (bronchus: primary); 46—liver (bronchus: tertiary); 47—lung (parenchyma); 48—lymph gland (tonsil); 49—muscle (skeletal); 50—esophagus; 51—ovary; 52—pancreas; 53—pineal gland; 54—pituitary gland; 55—placenta; 56—prostate; 57—rectum; 58—skin (foreskin); 69—spinal cord; 60—spleen (parenchyma); 61—stomach (antrum); 62—stomach (body); 63—stomach (fundus); 64—stomach (pyloric canal); 65—testis; 66—thyroid gland; 67—trachea; 68—umbilical cord; 69—ureter; 70—uterus (cervix); 71—uterus (myometrium); and 72—vas deferens.

### Example 22

### h5D8 in Combination with Cisplatin, Doxorubicin, or Paclitaxel in a Subcutaneous Tumor Implantation Model

The efficacy of h5D6 was evaluated in combination with cisplatin, doxorubicin, or paclitaxel in a subcutaneous implanted CT26 model. CT26 cells were cultured in RPMI-1640 medium supplemented with 10% fetal bovine serum and penicillin/streptomycin (both 100 μg/ml). 10,000 cells/mouse were implanted subcutaneously into BALB/c mice, and mice were treated with h5D8 (15 mg/kg) twice a week and/or one of cisplatin (10 mg/kg) once a week, paclitaxel (15 mg/kg) twice a week, doxorubicin (4 mg/kg) once a week. There were 12 mice per treatment cohort. Tumor volumes and mouse weights were measured using calipers twice a week.

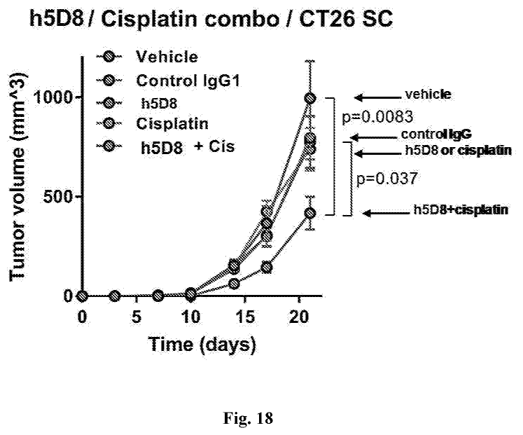

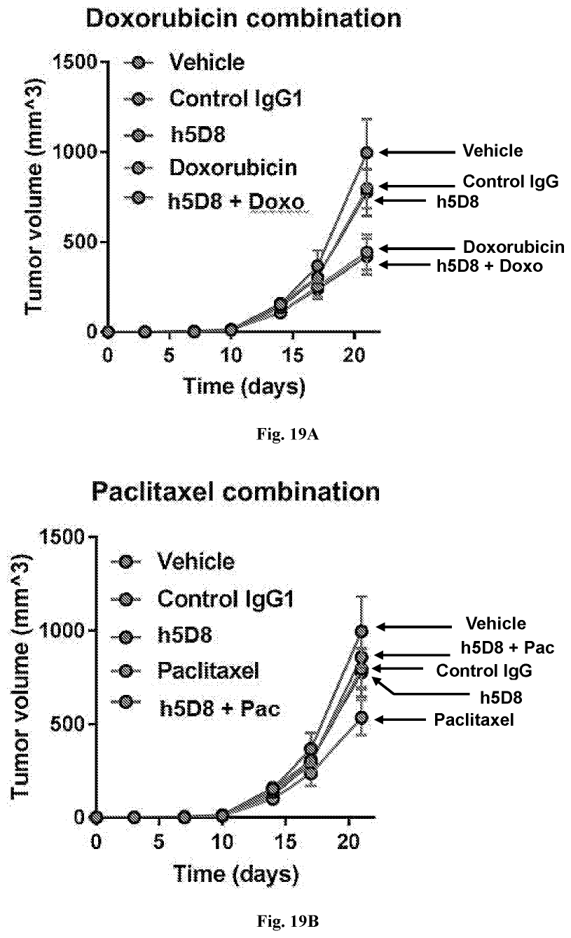

Mice treated with a combination of cisplatin and h5D8 exhibited decreased CT26 tumor growth when compared to mice treated with cisplatin or h5D8 alone, as shown in FIG. 18. Mice treated with a combination of doxorubicin and h5D8 did not show decreased CT26 tumor growth when compared to mice treated with doxorubicin or h5D8 alone, as shown in FIG. 19A. Mice treated with a combination of paclitaxel and h5D8 did not show decreased CT26 tumor growth when compared to mice treated with paclitaxel or h5D8 alone, as shown in FIG. 19B. The tumor volumes for all the animals in all treatment groups measured at the endpoint of day 21 are shown in Table 10.

TABLE 10
Tumor volume measured at endpoint (day 21)
Tumor Volumes at Endpoint (Day 21) [mm³ or cubic mm]

| | Control | | | | | h5D8 + | h5D8 + | h5D8 + |
|---|---|---|---|---|---|---|---|---|
| Vehicle* | IgG1\hat{ } | h5D8 | Doxorubicin | Cisplatin | Paclitaxel | Doxorubicin | Cisplatin*\hat{ } | Paclitaxel |
| 821.2 | 771.7 | 253.6 | 463.9 | 1313.6 | 762.1 | 205.3 | 588.9 | 1822.1 |
| 290.1 | 559.3 | 1408.5 | 588.8 | 676.0 | 1236.2 | 305.5 | 306.7 | 0.0 |
| 2455.3 | 1092.9 | 508.3 | 230.3 | 953.8 | 112.9 | 470.4 | 701.1 | 1488.2 |
| 724.5 | 695.5 | 656.8 | 408.7 | 328.5 | 651.8 | 303.1 | 258.7 | 261.8 |
| 1238.2 | 501.6 | 1354.4 | 1207.4 | 1279.7 | 214.0 | 481.7 | 855.2 | 751.6 |
| 551.1 | 336.0 | 1190.7 | 94.2 | 689.4 | 461.5 | 56.9 | 84.3 | 643.5 |
| 391.5 | 986.0 | 1109.1 | 109.1 | 512.3 | 698.3 | 633.1 | 328.3 | 691.6 |
| 791.7 | 509.0 | 906.6 | 294.9 | 193.9 | 457.7 | 333.0 | 117.8 | 658.0 |
| 1315.2 | 1092.5 | 640.3 | 780.2 | 997.5 | 256.2 | 389.2 | 742.9 | 533.9 |
| 1557.1 | 1415.4 | 0.0 | 351.2 | 257.9 | 592.2 | 387.5 | 482.6 | 737.1 |
| 814.5 | | 956.9 | 735.2 | 924.4 | 435.9 | 91.7 | 130.5 | 1001.5 |
| | | 318.7 | 48.9 | 744.2 | | 1394.3 | | 1712.8 |

*p = 0.0083 Vehicle vs h5D8 + Cisplatin
\hat{ }p = 0.037 Control IgG vs h5D8 + Cisplatin

### Example 23

### h5D8 and Cisplatin Combination in an Intradermal Tumor Implantation Model

The efficacy of h5D6 was evaluated in combination with cisplatin, doxorubicin, or paclitaxel in an intradermally implanted CT26 model. CT26 cells were cultured in RPMI-1640 medium supplemented with 10% fetal bovine serum and penicillin/streptomycin (both 100 μg/ml). 50,000 cells/mouse were implanted intradermally into BALB/c mice, and mice were treated with h5D8 (15 mg/kg) twice a week and/or cisplatin (10 mg/kg) once a week. There were 12 mice per treatment cohort. Tumor volumes and mouse weights were measured using calipers twice a week.

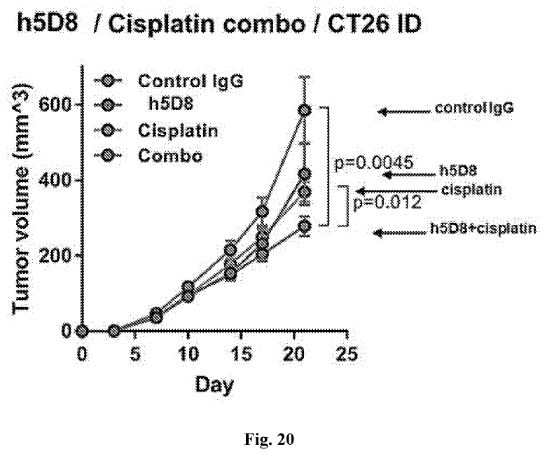

Mice treated with a combination of cisplatin and h5D8 exhibited decreased CT26 tumor growth when compared to mice treated with cisplatin or h5D8 alone, as shown in FIG. 20. The tumor volumes for all the animals in all treatment groups measured at the endpoint of day 21 are shown in Table 11.

TABLE 11
Tumor volume measured at endpoint (day 21)
Tumor Volumes at Endpoint (Day 21) [mm³ or cubic mm]

| Vehicle | Control | | | h5D8 + |
|---|---|---|---|---|
| Control | IgG* | h5D8 | Cisplatin\hat{ } | Cisplatin*\hat{ } |
| 513.2 | 349.9 | 233.8 | 408.2 | 344.5 |
| 454.2 | 798.5 | 137.3 | 333.3 | 425.0 |
| 1047.7 | 547.4 | 668.5 | 369.9 | 276.5 |
| 262.8 | 811.5 | 252.3 | 393.5 | 282.7 |
| 273.7 | 502.2 | 267.4 | 332.9 | 84.6 |
| 628.4 | 255.5 | 630.7 | 332.7 | 272.7 |
| 174.4 | 636.5 | 527.5 | 390.7 | 311.6 |
| 216.4 | 1165.2 | 182.7 | 574.5 | 215.6 |
| 1166.7 | 348.5 | 198.5 | 177.5 | 171.6 |
| | 946.1 | 1006.6 | 331.6 | 369.5 |
| | 557.3 | 471.5 | 335.3 | 287.4 |
| | 99.1 | | 445.5 | 296.2 |

*p = 0.0045 Control IgG vs h5D8 + Cisplatin
\hat{ }p = 0.012 Cisplatin vs h5D8 + Cisplatin

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

SEQUENCES

| SEQ | |
|---|---|
| ID | |
| NO | Sequence |
| 1 | GFTFSHAWMH |
| 2 | GFTFSHAW |
| 3 | HAWMH |
| 4 | QIKAKSDDYATYYAESVKG |
| 5 | IKAKSDDYAT |
| 6 | TCWEWDLDF |
| 7 | WEWDLDF |
| 8 | TSWEWDLDF |
| 9 | RSSQSLLDSDGHTYLN |
| 10 | QSLLDSDGHTY |
| 11 | SVSNLES |
| 12 | SVS |
| 13 | MQATHAPPYT |
| 14 | EVQLVESGGGLVKPGGSLKLSCAAS |
| 15 | QVQLQESGGGLVKPGGSLRLSCAAS |
| 16 | EVQLVESGGGVVQPGRSLRLSCAAS |
| 17 | EVQLMESGGGLVKPGGSLRLSCATS |
| 18 | WVRQAPGKGLEWVA |
| 19 | WVRQAPGKGLEWVG |
| 20 | RFTISRDDSKNTLYLQMNSLKTEDTAVYYC |
| 21 | RFSISRDNAKNSLYLQMNSLRVEDTVVYYC |
| 22 | RFTISRDDSKSTLFLQMNNLKTEDTAVYYC |
| 23 | WGQGTLVTVSS |
| 24 | WGQGTMVTVSS |
| 25 | WGQGTTVTVSS |
| 26 | DVVMTQSPLSLPVTLGQPASISC |
| 27 | DIVMTQTPLSSPVTLGQPASISC |
| 28 | DIVMTQTPLSLSVTPGQPASISC |
| 29 | DVVMTQSPLSQPVTLGQPASISC |
| 30 | WFQQRPGQSPRRLIY |
| 31 | WLQQRPGQPPRLLIY |
| 32 | WLLQKPGQPPQLLIY |
| 33 | WLQQRPGQSPRRLIY |
| 34 | GVPDRFSGSGSGTDFTLKISRVEAEDVGLYYC |
| 35 | GVPDRFSGSGAGTDFTLKISRVEAEDVGVYYC |
| 36 | GVPNRFSGSGSGTDFTLKISRVEAEDVGLYYC |
| 37 | GVPDRFNGSGSGTDFTLSISRVEAEDVGVYYC |
| 38 | FGQGTKLEIK |
| 39 | FGGGTKVEIK |
| 40 | FGQGTKVEIK |
| 41 | EVQLVESGGGLVKPGGSLKLSCAASGFTFSHAWMHWVRQAPGKGLEWVAQIKAKSDDYATYYAESVKGRFTISRD |
| | DSKNTLYLQMNSLKTEDTAVYYCTCWEWDLDFWGQGTLVTVSS |
| 42 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTCWEWDLDFWGQGTMVTVSS |
| 43 | EVQLVESGGGVVQPGRSLRLSCAASGFTFSHAWMHWVRQAPGKGLEWVAQIKAKSDDYATYYAESVKGRFSISR |
| | DNAKNSLYLQMNSLRVEDTVVYYCTCWEWDLDFWGQGTTVTVSS |
| 44 | EVQLMESGGGLVKPGGSLRLSCATSGFTFSHAWMHWVRQAPGKGLEWVGQIKAKSDDYATYYAESVKGRFTISR |
| | DDSKSTLFLQMNNLKTEDTAVYYCTCWEWDLDFWGQGTLVTVSS |
| 45 | DVVMTQSPLSLPVTLGQPASISCRSSQSLLDSDGHTYLNWFQQRPGQSPRRLIYSVSNLESGVPDRFSGSGSG |
| | TDFTLKISRVEAEDVGLYYCMQATHAPPYTFGQGTKLEIK |
| 46 | DIVMTQTPLSSPVTLGQPASISCRSSQSLLDSDGHTYLNWLQQRPGQPPRLLIYSVSNLESGVPDRFSGSGAGTDFTL |
| | KISRVEAEDVGVYYCMQATHAPPYTFGQGTKLEIK |
| 47 | DIVMTQTPLSLSVTPGQPASISCRSSQSLLDSDGHTYLNWLLQKPGQPPQLLIYSVSNLESGVPNRFSGSGSGTDFTL |
| | KISRVEAEDVGLYYCMQATHAPPYTFGGGTKVEIK |
| 48 | DVVMTQSPLSQPVTLGQPASISCRSSQSLLDSDGHTYLNWLQQRPGQSPRRLIYSVSNLESGVPDRFNGSGSGTDF |
| | TLSISRVEAEDVGVYYCMQATHAPPYTFGQGTKVEIK |
| 49 | MGWTLVFLFLLSVTAGVHSEVQLVESGGGLVKPGGSLKLSCAASGFTFSHAWMHWVRQAPGKGLEWVAQIKAK |
| | SDDYATYYAESVKGRFTISRDDSKNTLYLQMNSLKTEDTAVYYCTCWEWDLDFWGQGTLVTVSSASTKGPSVFPLA |
| | PSSKSTSGGTAALGCLVKDYFPEPVTVSWNSGALTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 50 | MGWTLVFLFLLSVTAGVHSQVQLQESGGGLVKPGGSLRLSCAASGFTFSHAWMHWVRQAPGKGLEWVGQIKAK |
| | SDDYATYYAESVKGRFTISRDDSKNTLYLQMNSLKTEDTAVYYCTCWEWDLDFWGQGTMVTVSSASTKGPSVFPL |
| | APSSKSTSGGTAALGCLVKDYFPEPVTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 51 | MGWTLVFLFLLSVTAGVHSEVQLVESGGGVVQPGRSLRLSCAASGFTFSHAWMHWVRQAPGKGLEWVAQIKAK |
| | SDDYATYYAESVKGRFSISRDNAKNSLYLQMNSLRVEDTVVYYCTCWEWDLDFWGQGTTVTVSSASTKGPSVFPL |
| | APSSKSTSGGTAALGCLVKDYFPEPVTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 52 | MGWTLVFLFLLSVTAGVHSEVQLMESGGGLVKPGGSLRLSCATSGFTFSHAWMHWVRQAPGKGLEWVGQIKAK |
| | SDDYATYYAESVKGRFTISRDDSKSTLFLQMNNLKTEDTAVYYCTCWEWDLDFWGQGTLVTVSSASTKGPSVFPLA |
| | PSSKSTSGGTAALGCLVKDYFPEPVTVSWNSGALTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 53 | MVSSAQFLGLLLLCFQGTRCDVVMTQSPLSLPVTLGQPASISCRSSQSLLDSDGHTYLNWFQQRPGQSPRRLIYSVS |
| | NLESGVPDRFSGSGSGTDFTLKISRVEAEDVGLYYCMQATHAPPYTFGQGTKLEIKRTVAAPSVFIFPPSDEQLKSGT |
| | ASVVCLLNNFYPREAKVQWKVDNALQSGNSQESVTEQDSKDSTYSLSSTLTLSKADYEKHKVYACEVTHQGLSSPV |
| | TKSFNRGEC |
| 54 | MVSSAQFLGLLLLCFQGTRCDIVMTQTPLSSPVTLGQPASISCRSSQSLLDSDGHTYLNWLQQRPGQPPRLLIYSVS |
| | NLESGVPDRFSGSGAGTDFTLKISRVEAEDVGVYYCMQATHAPPYTFGQGTKLEIKRTVAAPSVFIFPPSDEQLKSGT |
| | ASVVCLLNNFYPREAKVQWKVDNALQSGNSQESVTEQDSKDSTYSLSSTLTLSKADYEKHKVYACEVTHQGLSSPV |
| | TKSFNRGEC |
| 55 | MVSSAQFLGLLLLCFQGTRCDIVMTQTPLSLSVTPGQPASISCRSSQSLLDSDGHTYLNWLLQKPGQPPQLLIYSVSN |
| | LESGVPNRFSGSGSGTDFTLKISRVEAEDVGLYYCMQATHAPPYTFGGGTKVEIKRTVAAPSVFIFPPSDEQLKSGTA |
| | SVVCLLNNFYPREAKVQWKVDNALQSGNSQESVTEQDSKDSTYSLSSTLTLSKADYEKHKVYACEVTHQGLSSPVTK |
| | SFNRGEC |
| 56 | MVSSAQFLGLLLLCFQGTRCDVVMTQSPLSQPVTLGQPASISCRSSQSLLDSDGHTYLNWLQQRPGQSPRRLIYSVS |
| | NLESGVPDRFSGSGSGTDFTLSISRVEAEDVGVYYCMQATHAPPYTFGQGTKVEIKRTVAAPSVFIFPPSDEQLKSGT |
| | ASVVCLLNNFYPREAKVQWKVDNALQSGNSQESVTEQDSKDSTYSLSSTLTLSKADYEKHKVYACEVTHQGLSSPV |
| | TKSFNRGEC |
| 57 | EVQLVESGGGLVKPGGSLKLSCAASGFTFSHAWMHWVRQAPGKGLEWVAQIKAKSDDYATYYAESVKGRFTISRD |
| | DSKNTLYLQMNSLKTEDTAVYYCTCWEWDLDFWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSLTCLVKGFYPSDIAVEW |
| | ESNGQPENNYKTTPPVLDSDGSFFLYSKLTVDKSRWQQGNVFSCSVMHEALHNHYTQKSLSLSPGK |
| 58 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTCWEWDLDFWGQGTMVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKSLSLSPGK |
| 59 | EVQLVESGGGVVQPGRSLRLSCAASGFTFSHAWMHWVRQAPGKGLEWVAQIKAKSDDYATYYAESVKGRFSISR |
| | DNAKNSLYLQMNSLRVEDTVVYYCTCWEWDLDFWGQGTTVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 60 | EVQLMESGGGLVKPGGSLRLSCATSGFTFSHAWMHWVRQAPGKGLEWVGQIKAKSDDYATYYAESVKGRFTISR |
| | DDSKSTLFLQMNNLKTEDTAVYYCTCWEWDLDFWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 61 | DVVMTQSPLSLPVTLGQPASISCRSSQSLLDSDGHTYLNWFQQRPGQSPRRLIYSVSNLESGVPDRFSGSGSGTDFT |
| | LKISRVEAEDVGLYYCMQATHAPPYTFGQGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGEC |
| 62 | DIVMTQTPLSSPVTLGQPASISCRSSQSLLDSDGHTYLNWLQQRPGQPPRLLIYSVSNLESGVPDRFSGSGAGTDFTL |
| | KISRVEAEDVGVYYCMQATHAPPYTFGQGTKLEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPVTKSFNRGEC |
| 63 | DIVMTQTPLSLSVTPGQPASISCRSSQSLLDSDGHTYLNWLLQKPGQPPQLLIYSVSNLESGVPNRFSGSGSGTDFTL |
| | KISRVEAEDVGLYYCMQATHAPPYTFGGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPVTKSFNRGEC |
| 64 | DVVMTQSPLSQPVTLGQPASISCRSSQSLLDSDGHTYLNWLQQRPGQSPRRLIYSVSNLESGVPDRFSGSGSGTDFT |
| | LSISRVEAEDVGVYYCMQATHAPPYTFGQGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGEC |
| 65 | Blank |
| 66 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTSWEWDLDFWGQGTMVTVSS |
| 67 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTSWEWDLDFWGQGTMVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 68 | SPLPITPVNATCAIRHPCHNNLMNQIRSQLAQLNGSANALFILYYTAQGEPFPNNLDKLCGPNVTDFPPFHANGTEK |
| | AKLVELYRIVVYLGTSLGNITRDQKILNPSALSLHSKLNATADILRGLLSNVLCRLCSKYHVGHVDVTYGPDTSGKDVF |
| | QKKKLGCQLLGKYKQIIAVLAQAF |

What is claimed is:

1. A method of treating an individual with a cancer, comprising administering to the individual with the cancer an effective amount of:
a) a Leukemia Inhibitory Factor (LIF) binding polypeptide; and
b) a platinum-based antineoplastic agent.

2. The method of claim 1, comprising administering an effective amount of the LIF-binding polypeptide to the individual with cancer.

3. The method of claim 1, further comprising administering an effective amount of the platinum-based antineoplastic agent to the individual with cancer.

4. The method of claim 1, wherein the LIF-binding polypeptide comprises a fragment of an immunoglobulin variable region, or an immunoglobulin heavy chain constant region.

5. The method of claim 1, wherein the LIF-binding polypeptide comprises an antibody that specifically binds to LIF.

6. The method of claim 5, wherein the antibody that specifically binds to LIF comprises at least one framework region derived from a human antibody framework region.

7. The method of claim 5, wherein the antibody that specifically binds to LIF is humanized.

8. The method of claim 5, wherein the antibody that specifically binds to LIF is deimmunized.

9. The method of claim 5, wherein the antibody that specifically binds to LIF comprises two immunoglobulin heavy chains and two immunoglobulin light chains.

10. The method of claim 5, wherein the antibody that specifically binds to LIF is an IgG antibody.

11. The method of claim 5, wherein the antibody that specifically binds to LIF is a Fab, F(ab)₂, single-domain antibody, a single chain variable fragment (scFv), or a nanobody.

12. The method of claim 5, wherein the antibody that specifically binds to LIF comprises:
a) an immunoglobulin heavy chain complementarity determining region 1 (VH-CDR1) comprising the amino acid sequence set forth in any one of SEQ ID NOs: 1-3;
b) an immunoglobulin heavy chain complementarity determining region 2 (VH-CDR2) comprising the amino acid sequence set forth in any one of SEQ ID NOs: 4 or 5;
c) an immunoglobulin heavy chain complementarity determining region 3 (VH-CDR3) comprising the amino acid sequence set forth in any one of SEQ ID NOs: 6-8;
d) an immunoglobulin light chain complementarity determining region 1 (VL-CDR1) comprising the amino acid sequence set forth in any one of SEQ ID NOs: 9 or 10;
e) an immunoglobulin light chain complementarity determining region 2 (VL-CDR2) comprising the amino acid sequence set forth in any one of SEQ ID NOs: 11 or 12; and
f) an immunoglobulin light chain complementarity determining region 3 (VL-CDR3) comprising the amino acid sequence set forth in SEQ ID NO: 13.

13. The method of claim 5, wherein the antibody that specifically binds to LIF comprises:
a) an immunoglobulin heavy chain variable region (VH) sequence with the amino acid sequence at least about 80%, 90%, 95%, 97%, 98%, 99%, or 100% identical to the amino acid sequence set forth in any one of SEQ ID NOs: 41, 42, 44 or 66; and
b) an immunoglobulin light chain variable region (VL) sequence with the amino acid sequence at least about 80%, 90%, 95%, 97%, 98%, 99%, or 100% identical to the amino acid sequence set forth in any one of SEQ ID NOs: 45-48.

14. The method of claim 13, wherein the VH sequence is at least about 80%, 90%, 95%, 97%, 98%, 99%, or 100% identical to the amino acid sequence set forth in SEQ ID NO: 42; and the VL sequence is at least about 80%, 90%, 95%, 97%, 98%, 99%, or 100% identical to the amino acid sequence set forth in SEQ ID NO: 46.

15. The method of claim 14, wherein the VH sequence is identical to the amino acid sequence set forth in SEQ ID NO: 42; and the VL sequence is identical to the amino acid sequence set forth in SEQ ID NO: 46.

16. The method of claim 5, wherein the antibody that specifically binds to LIF comprises:
a) an immunoglobulin heavy chain sequence with the amino acid sequence at least about 80%, 90%, 95%, 97%, 98%, or 99% identical to the amino acid sequence set forth in any one of SEQ ID NOs: 57-60 or 67; and
b) an immunoglobulin light chain sequence with the amino acid sequence at least about 80%, 90%, 95%, 97%, 98%, or 99% identical to the amino acid sequence set forth in any one of SEQ ID NOs: 61-64.

17. The method of claim 5, wherein the antibody that specifically binds to LIF binds with a K_{D} of less than about 200 picomolar.

18. The method of claim 5, wherein the antibody that specifically binds to LIF binds with a K_{D} of less than about 100 picomolar.

19. The method of claim 1, wherein the platinum-based antineoplastic agent comprises cisplatin, carboplatin, oxaliplatin, nedaplatin, triplatin tetranitrate, phenathriplatin, picoplatin, satraplatin, or combinations thereof.

20. The method of claim 19, wherein the platinum-based antineoplastic agent is cisplatin.

21. The method of claim 1, wherein the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer.

22. The method of claim 21, wherein the cancer comprises non-small cell lung cancer, epithelial ovarian carcinoma, or pancreatic adenocarcinoma.

23. The method of claim 1, wherein the cancer is refractory to treatment with a therapeutic amount of an inhibitor of a LIF-binding polypeptide.

24. The method of claim 1, wherein the cancer is refractory to treatment with a therapeutic amount of a platinum-based antineoplastic agent.

25. The method of claim 1, wherein the Leukemia Inhibitory Factor (LIF) binding polypeptide and the platinum-based antineoplastic agent are administered separately.

26. The method of claim 1, wherein the LIF-binding polypeptide and the platinum-based antineoplastic agent are administered on different schedules.

27. The method of claim 1, wherein the LIF-binding polypeptide and the platinum-based antineoplastic agent are administered in a single composition.

28. A method of treating an individual with a cancer comprising administering to the individual with cancer an effective amount of a combination of:
a) of an antibody that specifically binds Leukemia Inhibitory Factor (LIF) comprising:
i. an immunoglobulin heavy chain complementarity determining region 1 (VH-CDR1) comprising the amino acid sequence set forth in any one of SEQ ID NOs: 1-3;
ii. an immunoglobulin heavy chain complementarity determining region 2 (VH-CDR2) comprising the amino acid sequence set forth in any one of SEQ ID NOs: 4or5;
iii. an immunoglobulin heavy chain complementarity determining region 3 (VH-CDR3) comprising the amino acid sequence set forth in any one of SEQ ID NOs: 6-8;
iv. an immunoglobulin light chain complementarity determining region 1 (VL-CDR1) comprising the amino acid sequence set forth in any one of SEQ ID NOs: 9 or 10;
v. an immunoglobulin light chain complementarity determining region 2 (VL-CDR2) comprising the amino acid sequence set forth in any one of SEQ ID NOs: 11 or 12; and
vi. an immunoglobulin light chain complementarity determining region 3 (VL-CDR3) comprising the amino acid sequence set forth in SEQ ID NO: 13; and
b) a platinum-based antineoplastic agent.

29. The method of claim 28, further comprising administering an effective amount of the LIF-binding polypeptide to the individual with cancer.

30. The method of claim 28, further comprising administering an effective amount of the platinum-based antineoplastic agent to the individual with cancer.


SEQUENCE LISTING (68 sequences)

SEQ ID NO: 1 - protein, 10 aa, Artificial Sequence
  Synthetic Peptide
GFTFSHAWMH

SEQ ID NO: 2 - protein, 8 aa, Artificial Sequence
  Synthetic Peptide
GFTFSHAW

SEQ ID NO: 3 - protein, 5 aa, Artificial Sequence
  Synthetic Peptide
HAWMH

SEQ ID NO: 4 - protein, 19 aa, Artificial Sequence
  Synthetic Peptide
QIKAKSDDYATYYAESVKG

SEQ ID NO: 5 - protein, 10 aa, Artificial Sequence
  Synthetic Peptide
IKAKSDDYAT

SEQ ID NO: 6 - protein, 9 aa, Artificial Sequence
  Synthetic Peptide
TCWEWDLDF

SEQ ID NO: 7 - protein, 7 aa, Artificial Sequence
  Synthetic Peptide
WEWDLDF

SEQ ID NO: 8 - protein, 9 aa, Artificial Sequence
  Synthetic Peptide
TSWEWDLDF

SEQ ID NO: 9 - protein, 16 aa, Artificial Sequence
  Synthetic Peptide
RSSQSLLDSDGHTYLN

SEQ ID NO: 10 - protein, 11 aa, Artificial Sequence
  Synthetic Peptide
QSLLDSDGHTY

SEQ ID NO: 11 - protein, 7 aa, Artificial Sequence
  Synthetic Peptide
SVSNLES

SEQ ID NO: 12 - protein, 3 aa, Artificial Sequence
  Synthetic Peptide
SVS

SEQ ID NO: 13 - protein, 10 aa, Artificial Sequence
  Synthetic Peptide
MQATHAPPYT

SEQ ID NO: 14 - protein, 25 aa, Artificial Sequence
  Synthetic Peptide
EVQLVESGGGLVKPGGSLKLSCAAS

SEQ ID NO: 15 - protein, 25 aa, Artificial Sequence
  Synthetic Peptide
QVQLQESGGGLVKPGGSLRLSCAAS

SEQ ID NO: 16 - protein, 25 aa, Artificial Sequence
  Synthetic Peptide
EVQLVESGGGVVQPGRSLRLSCAAS

SEQ ID NO: 17 - protein, 25 aa, Artificial Sequence
  Synthetic Peptide
EVQLMESGGGLVKPGGSLRLSCATS

SEQ ID NO: 18 - protein, 14 aa, Artificial Sequence
  Synthetic Peptide
WVRQAPGKGLEWVA

SEQ ID NO: 19 - protein, 14 aa, Artificial Sequence
  Synthetic Peptide
WVRQAPGKGLEWVG

SEQ ID NO: 20 - protein, 30 aa, Artificial Sequence
  Synthetic Peptide
RFTISRDDSKNTLYLQMNSLKTEDTAVYYC

SEQ ID NO: 21 - protein, 30 aa, Artificial Sequence
  Synthetic Peptide
RFSISRDNAKNSLYLQMNSLRVEDTVVYYC

SEQ ID NO: 22 - protein, 30 aa, Artificial Sequence
  Synthetic Peptide
RFTISRDDSKSTLFLQMNNLKTEDTAVYYC

SEQ ID NO: 23 - protein, 11 aa, Artificial Sequence
  Synthetic Peptide
WGQGTLVTVSS

SEQ ID NO: 24 - protein, 11 aa, Artificial Sequence
  Synthetic Peptide
WGQGTMVTVSS

SEQ ID NO: 25 - protein, 11 aa, Artificial Sequence
  Synthetic Peptide
WGQGTTVTVSS

SEQ ID NO: 26 - protein, 23 aa, Artificial Sequence
  Synthetic Peptide
DVVMTQSPLSLPVTLGQPASISC

SEQ ID NO: 27 - protein, 23 aa, Artificial Sequence
  Synthetic Peptide
DIVMTQTPLSSPVTLGQPASISC

SEQ ID NO: 28 - protein, 23 aa, Artificial Sequence
  Synthetic Peptide
DIVMTQTPLSLSVTPGQPASISC

SEQ ID NO: 29 - protein, 23 aa, Artificial Sequence
  Synthetic Peptide
DVVMTQSPLSQPVTLGQPASISC

SEQ ID NO: 30 - protein, 15 aa, Artificial Sequence
  Synthetic Peptide
WFQQRPGQSPRRLIY

SEQ ID NO: 31 - protein, 15 aa, Artificial Sequence
  Synthetic Peptide
WLQQRPGQPPRLLIY

SEQ ID NO: 32 - protein, 15 aa, Artificial Sequence
  Synthetic Peptide
WLLQKPGQPPQLLIY

SEQ ID NO: 33 - protein, 15 aa, Artificial Sequence
  Synthetic Peptide
WLQQRPGQSPRRLIY

SEQ ID NO: 34 - protein, 32 aa, Artificial Sequence
  Synthetic Peptide
GVPDRFSGSGSGTDFTLKISRVEAEDVGLYYC

SEQ ID NO: 35 - protein, 32 aa, Artificial Sequence
  Synthetic Peptide
GVPDRFSGSGAGTDFTLKISRVEAEDVGVYYC

SEQ ID NO: 36 - protein, 32 aa, Artificial Sequence
  Synthetic Peptide
GVPNRFSGSGSGTDFTLKISRVEAEDVGLYYC

SEQ ID NO: 37 - protein, 32 aa, Artificial Sequence
  Synthetic Peptide
GVPDRFNGSGSGTDFTLSISRVEAEDVGVYYC

SEQ ID NO: 38 - protein, 10 aa, Artificial Sequence
  Synthetic Peptide
FGQGTKLEIK

SEQ ID NO: 39 - protein, 10 aa, Artificial Sequence
  Synthetic Peptide
FGGGTKVEIK

SEQ ID NO: 40 - protein, 10 aa, Artificial Sequence
  Synthetic Peptide
FGQGTKVEIK

SEQ ID NO: 41 - protein, 118 aa, Artificial Sequence
  Synthetic Peptide
EVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSS

SEQ ID NO: 42 - protein, 118 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSS

SEQ ID NO: 43 - protein, 118 aa, Artificial Sequence
  Synthetic Peptide
EVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSS

SEQ ID NO: 44 - protein, 118 aa, Artificial Sequence
  Synthetic Peptide
EVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSS

SEQ ID NO: 45 - protein, 113 aa, Artificial Sequence
  Synthetic Peptide
DVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIK

SEQ ID NO: 46 - protein, 113 aa, Artificial Sequence
  Synthetic Peptide
DIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIK

SEQ ID NO: 47 - protein, 113 aa, Artificial Sequence
  Synthetic Peptide
DIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIK

SEQ ID NO: 48 - protein, 113 aa, Artificial Sequence
  Synthetic Peptide
DVVMTQSPLSQPVTLGQPASISCRSSQSLLDSDGHTYLNWLQQRPGQSPRRLIYSVSNLESGVPDRFNGSGSGTDFTLSISRVEAEDVGVYYCMQATHAPPYTFGQGTKVEIK

SEQ ID NO: 49 - protein, 467 aa, Artificial Sequence
  Synthetic Peptide
MGWTLVFLFLLSVTAGVHSEVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 467 aa, Artificial Sequence
  Synthetic Peptide
MGWTLVFLFLLSVTAGVHSQVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 467 aa, Artificial Sequence
  Synthetic Peptide
MGWTLVFLFLLSVTAGVHSEVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 467 aa, Artificial Sequence
  Synthetic Peptide
MGWTLVFLFLLSVTAGVHSEVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 240 aa, Artificial Sequence
  Synthetic Peptide
MVSSAQFLGLLLLCFQGTRCDVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 240 aa, Artificial Sequence
  Synthetic Peptide
MVSSAQFLGLLLLCFQGTRCDIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 240 aa, Artificial Sequence
  Synthetic Peptide
MVSSAQFLGLLLLCFQGTRCDIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 240 aa, Artificial Sequence
  Synthetic Peptide
MVSSAQFLGLLLLCFQGTRCDVVMTQSPLSQPVTLGQPASISCRSSQSLLDSDGHTYLNWLQQRPGQSPRRLIYSVSNLESGVPDRFSGSGSGTDFTLSISRVEAEDVGVYYCMQATHAPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 448 aa, Artificial Sequence
  Synthetic Peptide
EVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 448 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 448 aa, Artificial Sequence
  Synthetic Peptide
EVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 448 aa, Artificial Sequence
  Synthetic Peptide
EVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 220 aa, Artificial Sequence
  Synthetic Peptide
DVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 220 aa, Artificial Sequence
  Synthetic Peptide
DIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 220 aa, Artificial Sequence
  Synthetic Peptide
DIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 220 aa, Artificial Sequence
  Synthetic Peptide
DVVMTQSPLSQPVTLGQPASISCRSSQSLLDSDGHTYLNWLQQRPGQSPRRLIYSVSNLESGVPDRFSGSGSGTDFTLSISRVEAEDVGVYYCMQATHAPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - skipped

SEQ ID NO: 66 - protein, 118 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTSWEWDLDFWGQGTMVTVSS

SEQ ID NO: 67 - protein, 448 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTSWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 180 aa, Artificial Sequence
  Synthetic Peptide
SPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF